(19) United States
(12) Patent Application Publication
(10) Pub. No.: US 2025/0032388 A1
(43) Pub. Date: Jan. 30, 2025

(54) PERSONAL CARE COMPOSITIONS FOR SENSITIVE SKIN AND METHODS OF USE

(71) Applicant: Galderma Holding SA, ZUG (CH)

(72) Inventors: Ruandro Victor KNAPIK CECCON, Montreal (CA); Nicolas JOLY-TONETTI, Tatroz (CH); Virginie LE NOEL, Lugrin (FR); Sandrine Therese Beatrice PASCAL-SUISSE, Chassieu (FR); Nadège Maryline LACHMANN, La Tour de Peilz (CH); Mathieu GRIVET-SEYVE, Southlake, TX (US); Hyun Dae CHO, Cheong ju si (KR); Jae Yong SEO, Cheongju-si (KR)

(21) Appl. No.: 18/899,935

(22) Filed: Sep. 27, 2024

Related U.S. Application Data
(63) Continuation of application No. PCT/US2023/016994, filed on Mar. 30, 2023.
(60) Provisional application No. 63/325,960, filed on Mar. 31, 2022.

(51) Int. Cl. A61K 8/67; A61K 8/19; A61K 8/27; A61K 8/29; A61K 8/9789; A61K 8/9794; A61Q 17/04; A61Q 19/00

(52) U.S. Cl. CPC A61K 8/675; A61K 8/19; A61K 8/27; A61K 8/29; A61K 8/673; A61K 8/9789; A61K 8/9794; A61Q 17/04; A61Q 19/005

(57) ABSTRACT

Provided herein are personal care compositions for reducing the signs of aging in sensitive skin without the irritating effects associated with retinol. Such personal care compositions comprise niacinamide, Leontopodium alpinum extract, Oryza sativa lees extract, panthenol, and soy-derived glycopeptides. Use of such compositions also modulates some biochemical indicators that are associated with premature aging of skin in response to pollution.

### CROSS-REFERENCE

This application is a continuation of International application PCT/US2023/016994 filed Mar. 30, 2023, which application claims the benefit of U.S. Provisional Patent Application No. 63/325,960 filed on Mar. 31, 2022, all of which applications are hereby incorporated by reference in their entireties.

### FIELD OF DISCLOSURE

The present disclosure relates generally to the field of skin care. Included are personal care compositions that protect sensitive skin from environmental challenges (e.g., pollution), restore a healthy balance (e.g., of skin pH, skin humidity, etc.) and ameliorate the signs of aging. This disclosure also provides methods for treating sensitive skin in a subject using the personal care compositions described herein.

### BACKGROUND

Skin is the first barrier against environmental challenges including artificial blue light, particulate matter and ultraviolet (UV) light. Environmental challenges damage skin over time and accelerate skin aging.

Sensitive skin is described as an altered response to environmental factors resulting in unpleasant sensations, such as tingling, heat, burning, itching, tightness and erythema, affecting all locations of the body, especially the face. Sensitive skin shows inflammation, specifically altered skin barrier, thickened epidermis with hyperplasia, disorganized collagen, and reduced vascular network, which makes it more susceptible to damage on the surface and in lower layers, and shows neurosensitivity, resulting in faster aging of sensitive skin compared to normal skin.

Anti-aging lotions typically contain retinoids which stimulate collagen I synthesis and reduce wrinkles to maintain a healthy and youthful-looking skin but retinoids can also irritate sensitive skin making it more sensitive.

There is an unmet need for compositions that slow down or reduce the signs of sensitive skin aging.

### SUMMARY

In one aspect, which may be combined with any other aspect or embodiment, the disclosure relates to a personal care composition comprising, by weight of the total composition:
(a) 0.1%-10% niacinamide,
(b) 0.0001%-10% Edelweiss (Leontopodium alpinum) extract,
(c) 0.001%-10% rice (Oryza sativa) lees concentrate,
(d) 0.1%-10% panthenol, and
(e) 0.001%-5% soy-derived glycopeptides.

In some embodiments, the personal care composition comprises, by weight of the total composition: 0.1%-4% niacinamide, 0.001%-4% Edelweiss (Leontopodium alpinum), 0.001%-2% rice (Oryza sativa) lees concentrate, 0.1%-3% panthenol, and 0.002%-4% soy-derived glycopeptides.

In some embodiments, the personal care composition does not comprise any retinoid or retinoid derivatives.

In some embodiments, the personal care composition further comprises at least one of the following: water, adenosine, a moisturizing agent, a light protective filter, an antioxidant, a preservative, a gelling agent, a solvent, a pH balancing agent, and a surfactant.

In some embodiments, the moisturizing agent comprises at least one of Buddleja davidii extract, Thymus vulgaris extract, caprylyl glycol, glycerin, 1,2-hexanediol, butylene glycol, or any combination thereof.

In some embodiments, the personal care composition further comprises, by weight of the total composition: 0.01%-2% adenosine, 0.0001%-3% Buddleja davidii extract, and 0.0001%-3% Thymus vulgaris extract.

In some embodiments, the light protective filter comprises at least one of zinc oxide, titanium dioxide and iron oxide.

In some embodiments, the personal care composition is a cream, a lotion, a gel, a mask, a serum, or a spray.

In another aspect, which may be combined with any other aspect or embodiment, the present disclosure relates to a method for treating sensitive skin in a subject, the method comprising administering to the subject a personal care composition comprising: niacinamide, Edelweiss (Leontopodium alpinum) extract, rice (Oryza sativa) lees concentrate, panthenol, and soy-derived glycopeptides.

In some embodiments, the sensitive skin exhibits at least one unpleasant sensation before the treatment, the at least one unpleasant sensation selected from the group consisting of tingling, heat, burning, itching, tightness and erythema.

In some embodiments, the administration of the personal care composition results in a reduction in wrinkles, or a delay in the onset of wrinkles. In some embodiments, the administration of the personal care composition reduces the signs of ageing by at least 3%, 5%, 10%, 15%, 20%, 25%, 30%, 35%, 40%, 50%, 60%, 70%, 80%, 90%, 95%, 99% or more as compared to administration of the same composition without niacinamide, Edelweiss (Leontopodium alpinum) extract, rice (Oryza sativa) lees concentrate, panthenol, and soy-derived glycopeptides.

In some embodiments, the personal care composition does not comprise any retinoid or retinoid derivatives.

In some embodiments, the personal care composition comprises, by weight of the total composition:
(a) 0.1%-10% niacinamide,
(b) 0.0001%-10% Edelweiss (Leontopodium alpinum) extract,
(c) 0.001%-10% rice (Oryza sativa) lees concentrate,
(d) 0.1%-10% panthenol, and
(e) 0.001%-5% soy-derived glycopeptides.

In some embodiments, the personal care composition comprises, by weight of the total composition: 0.1%-4% niacinamide, 0.001%-4% Edelweiss (Leontopodium alpinum), 0.001%-2% rice (Oryza sativa) lees concentrate, 0.1%-3% panthenol, and 0.002%-4% soy-derived glycopeptides.

In some embodiments, the personal care composition does not comprise any retinoid or retinoid derivatives.

In some embodiments, the personal care composition further comprises at least one of the following: water, adenosine, a moisturizing agent, a light protective filter, an antioxidant, a preservative, a gelling agent, a solvent, a pH balancing agent, and a surfactant.

In some embodiments, the moisturizing agent comprises at least one of Buddleja davidii extract, Thymus vulgaris extract, caprylyl glycol, glycerin, 1,2-hexanediol, butylene glycol, or any combination thereof.

In some embodiments, the personal care composition further comprises, by weight of the total composition: 0.01%-2% adenosine, 0.0001%-3% Buddleja davidii extract, and 0.0001%-3% Thymus vulgaris extract.

In some embodiments, the light protective filter comprises at least one of zinc oxide, titanium dioxide and iron oxide.

In some embodiments, the personal care composition is a cream, a lotion, a gel, a mask, a serum, or a spray.

In another aspect, which may be combined with any other aspect or embodiment, the present disclosure relates to a personal care composition comprising, by weight of the total composition:
(a) 0.1%-10% niacinamide,
(b) 0.0001%-10% Edelweiss (Leontopodium alpinum) extract,
(c) 0.001%-10% rice (Oryza sativa) lees concentrate, and
(d) 0.1%-10% panthenol.

In some embodiments, the personal care composition further comprises: (e) 0.001%-5% soy-derived glycopeptides.

In some embodiments, the personal care composition comprises, by weight of the total composition: 0.1%-4% niacinamide, 0.001%-4% Edelweiss (Leontopodium alpinum), 0.001%-2% rice (Oryza sativa) lees concentrate, 0.1%-3% panthenol, and 0.002%-4% soy-derived glycopeptides. In some embodiments, the personal care composition does not comprise any retinoid or retinoid derivatives.

In some embodiments, the personal care composition further comprises at least one of the following: water, adenosine, a moisturizing agent, a light protective filter, an antioxidant, a preservative, a gelling agent, a solvent, a pH balancing agent, and a surfactant.

In some embodiments, the moisturizing agent comprises at least one of Buddleja davidii extract, Thymus vulgaris extract, caprylyl glycol, glycerin, 1,2-hexanediol, butylene glycol, or any combination thereof.

In some embodiments, the personal care composition further comprises, by weight of the total composition: 0.01%-2% adenosine, 0.0001%-3% Buddleja davidii extract, and 0.0001%-3% Thymus vulgaris extract.

In some embodiments, the light protective filter comprises at least one of zinc oxide, titanium dioxide and iron oxide.

In some embodiments, the personal care composition is a cream, a lotion, a gel, a mask, a serum, or a spray.

In another aspect, which may be combined with any other aspect or embodiment, the present disclosure relates to a method for reducing the onset of wrinkles in a subject with sensitive skin, the method comprising administering to the subject a personal care composition comprising niacinamide, Edelweiss (Leontopodium alpinum) extract, rice (Oryza sativa) lees concentrate, panthenol and soy-derived glycopeptides.

In some embodiments, the method further comprises exposing the subject's skin to pollution following treatment with the compositions disclosed herein.

In some embodiments, the subject's skin has been exposed to pollution.

In some embodiments, the pollution is air pollution. In some embodiments, the air pollution has an Air Quality Index (AQI) of above 100, 150, 200, 250 or 300. In some embodiments, the pollution is water pollution.

In some embodiments, the personal care composition does not comprise any retinoid or retinoid derivatives.

In some embodiments, the personal care composition comprises, by weight by weight of the total composition:
(a) 0.1%-10% niacinamide,
(b) 0.0001%-10% Edelweiss (Leontopodium alpinum) extract,
(c) 0.001%-10% rice (Oryza sativa) lees concentrate,
(d) 0.1%-10% panthenol, and
(e) 0.001%-5% soy-derived glycopeptides.

In some embodiments, the administration of the personal care composition results in a reduction in wrinkles, or a delay in the onset of wrinkles. In some embodiments, the administration of the personal care composition reduces the signs of ageing by at least 5%, 10%, 15%, 20%, 25%, 30%, 35%, 40%, 50%, 60%, 70%, 80%, 90%, 95%, 99% or more as compared to administration of the same composition without niacinamide, Edelweiss (Leontopodium alpinum) extract, rice (Oryza sativa) lees concentrate, panthenol and soy-derived glycopeptides.

In some embodiments, the subject comprises a human. In some embodiments, the subject has been exposed to pollution. In some embodiments, the subject has been exposed to air pollution. In some embodiments, the subject has been exposed to air pollution having an Air Quality Index (AQI) of above 100. In some embodiments, the subject is exposed to air pollution having an Air Quality Index (AQI) of above 150. In some embodiments, the subject is exposed to air pollution having an Air Quality Index (AQI) of above 200. In some embodiments, the subject is exposed to air pollution having an Air Quality Index (AQI) of above 250. In some embodiments, the subject is exposed to air pollution having an Air Quality Index (AQI) of above 300.

In some embodiments, the personal care composition comprises, by weight of the total composition: 0.1%-4% niacinamide, 0.001%-4% Edelweiss (Leontopodium alpinum), 0.001%-2% rice (Oryza sativa) lees concentrate, 0.1%-3% panthenol, and 0.002%-4% soy-derived glycopeptides.

In some embodiments, the personal care composition does not comprise any retinoid or retinoid derivatives.

In some embodiments, the personal care composition further comprises at least one of the following: water, adenosine, a moisturizing agent, a light protective filter, an antioxidant, a preservative, a gelling agent, a solvent, a pH balancing agent, and a surfactant.

In some embodiments, the moisturizing agent comprises at least one of Buddleja davidii extract, Thymus vulgaris extract, caprylyl glycol, glycerin, 1,2-hexanediol, butylene glycol, or any combination thereof.

In some embodiments, the personal care composition further comprises, by weight of the total composition: 0.01%-2% adenosine, 0.0001%-3% Buddleja davidii extract, and 0.0001%-3% Thymus vulgaris extract.

In some embodiments, the light protective filter comprises at least one of zinc oxide, titanium dioxide and iron oxide.

In some embodiments, the personal care composition is a cream, a lotion, a gel, a mask, a serum, or a spray.

In some embodiments, the personal care composition is administered topically. In some embodiments, the personal care composition is administered every hour, every 2 hours, every 3 hours, every 6 hours, every 12 hours, or every day, every other day, or every three days. In some embodiments, the personal care composition is administered for at least 10, 11, 12, 13, 14, 15, 16, 17, 18, 19 or 20 days before a reduction in signs of aging of sensitive skin (reduction of wrinkles, e.g., in the crow's feet area, increased) or a reduction in at least one unpleasant sensation of sensitive skin (e.g., tingling, heat, burning, itching, tightness and erythema) is observed.

In another aspect, which may be combined with any other aspect or embodiment, the present disclosure relates to a method of reducing pollution induced NADPH oxidase in the mitochondria by the application of a composition comprising niacinamide, Leontopodium alpinum extract, rice (Oryza sativa) lees concentrate, and panthenol, to human skin.

In some embodiments, the composition further comprises at least one of the following: water, adenosine, a moisturizing agent, a light protective filter, an antioxidant, a preservative, a gelling agent, a solvent, a pH balancing agent, and a surfactant.

In some embodiments, the moisturizing agent comprises at least one of Buddleja davidii extract, Thymus vulgaris extract, caprylyl glycol, glycerin, 1,2-hexanediol, butylene glycol, or any combination thereof.

In some embodiments, the personal care composition further comprises, by weight of the total composition: 0.01%-2% adenosine, 0.0001%-3% Buddleja davidii extract, and 0.0001%-3% Thymus vulgaris extract.

In some embodiments, the light protective filter comprises at least one of zinc oxide, titanium dioxide and iron oxide.

In another aspect, which may be combined with any other aspect or embodiment, the present disclosure relates to a method of reducing pollution-induced fragmentation of mitochondria by the application of a composition comprising niacinamide, Leontopodium alpinum extract, rice (Oryza sativa) lees concentrate, and panthenol, to human skin.

In some embodiments, the composition further comprises at least one of the following: water, adenosine, a moisturizing agent, a light protective filter, an antioxidant, a preservative, a gelling agent, a solvent, a pH balancing agent, and a surfactant.

In some embodiments, the moisturizing agent comprises at least one of Buddleja davidii extract, Thymus vulgaris extract, caprylyl glycol, glycerin, 1,2-hexanediol, butylene glycol, or any combination thereof.

In some embodiments, the personal care composition further comprises, by weight of the total composition: 0.01%-2% adenosine, 0.0001%-3% Buddleja davidii extract, and 0.0001%-3% Thymus vulgaris extract.

In some embodiments, the light protective filter comprises at least one of zinc oxide, titanium dioxide and iron oxide.

In some embodiments, the composition further comprises at least one of the following: water, adenosine, a moisturizing agent, a light protective filter, an antioxidant, a preservative, a gelling agent, a solvent, a pH balancing agent, and a surfactant.

In some embodiments, the moisturizing agent comprises at least one of Buddleja davidii extract, Thymus vulgaris extract, caprylyl glycol, glycerin, 1,2-hexanediol, butylene glycol, or any combination thereof.

In some embodiments, the light protective filter comprises at least one of zinc oxide, titanium dioxide and iron oxide.

In another aspect, which may be combined with any other aspect or embodiment, the present disclosure relates to a method of reducing pollution induced production of MAMs in the mitochondria by the application of a composition comprising niacinamide, Leontopodium alpinum extract, rice (Oryza sativa) lees concentrate, and panthenol, to human skin.

In some embodiments, the composition further comprises at least one of the following: water, adenosine, a moisturizing agent, a light protective filter, an antioxidant, a preservative, a gelling agent, a solvent, a pH balancing agent, and a surfactant.

In some embodiments, the moisturizing agent comprises at least one of Buddleja davidii extract, Thymus vulgaris extract, caprylyl glycol, glycerin, 1,2-hexanediol, butylene glycol, or any combination thereof.

In some embodiments, the personal care composition further comprises, by weight of the total composition: 0.01%-2% adenosine, 0.0001%-3% Buddleja davidii extract, and 0.0001%-3% Thymus vulgaris extract.

In some embodiments, the light protective filter comprises at least one of zinc oxide, titanium dioxide and iron oxide.

In another aspect, which may be combined with any other aspect or embodiment, the present disclosure relates to a method of alleviating pollution induced mitochondrial membrane depolarization by the application of the composition of a composition comprising niacinamide, Leontopodium alpinum extract, rice (Oryza sativa) lees concentrate, and panthenol, to human skin.

In some embodiments, the composition further comprises at least one of the following: water, adenosine, a moisturizing agent, a light protective filter, an antioxidant, a preservative, a gelling agent, a solvent, a pH balancing agent, and a surfactant.

In some embodiments, the moisturizing agent comprises at least one of Buddleja davidii extract, Thymus vulgaris extract, caprylyl glycol, glycerin, 1,2-hexanediol, butylene glycol, or any combination thereof.

In some embodiments, the personal care composition further comprises, by weight of the total composition: 0.01%-2% adenosine, 0.0001%-3% Buddleja davidii extract, and 0.0001%-3% Thymus vulgaris extract.

In some embodiments, the light protective filter comprises at least one of zinc oxide, titanium dioxide and iron oxide.

In another aspect, which may be combined with any other aspect or embodiment, the present disclosure relates to a method of inducing pro-collagen and elastin protein expression in skin by topical application of a composition comprising: niacinamide, Edelweiss (Leontopodium alpinum) extract, rice (Oryza sativa) lees concentrate, and panthenol to the skin wherein the personal care composition does not include retinol.

In some embodiments, the composition further comprises at least one of the following: water, adenosine, a moisturizing agent, a light protective filter, an antioxidant, a preservative, a gelling agent, a solvent, a pH balancing agent, and a surfactant.

In some embodiments, the moisturizing agent comprises at least one of Buddleja davidii extract, Thymus vulgaris extract, caprylyl glycol, glycerin, 1,2-hexanediol, butylene glycol, or any combination thereof.

In some embodiments, the personal care composition further comprises, by weight of the total composition: 0.01%-2% adenosine, 0.0001%-3% Buddleja davidii extract, and 0.0001%-3% Thymus vulgaris extract.

In some embodiments, the light protective filter comprises at least one of zinc oxide, titanium dioxide and iron oxide.

In another aspect, which may be combined with any other aspect or embodiment, the present disclosure relates to a method of reducing IL-8 and PGE-2 expression in skin by topical application of a composition comprising: niacinamide, Edelweiss (Leontopodium alpinum) extract, rice (Oryza sativa) lees concentrate, and panthenol to the skin, wherein the personal care composition does not include retinol.

In some embodiments, the composition further comprises at least one of the following: water, adenosine, a moisturizing agent, a light protective filter, an antioxidant, a preservative, a gelling agent, a solvent, a pH balancing agent, and a surfactant.

In some embodiments, the moisturizing agent comprises at least one of Buddleja davidii extract, Thymus vulgaris extract, caprylyl glycol, glycerin, 1,2-hexanediol, butylene glycol, or any combination thereof.

In some embodiments, the personal care composition further comprises, by weight of the total composition: 0.01%-2% adenosine, 0.0001%-3% Buddleja davidii extract, and 0.0001%-3% Thymus vulgaris extract.

In some embodiments, the light protective filter comprises at least one of zinc oxide, titanium dioxide and iron oxide.

### BRIEF DESCRIPTION OF THE DRAWINGS

FIG. 1 shows the results of a skin sensitivity questionnaire with subjects using an embodiment of a composition according to the disclosure.

FIG. 2 shows the results of a TEWL/Hydration assay on subjects using an embodiment of a personal care composition according to the disclosure.

FIG. 3 shows the results of clinical scoring of skins treated with an embodiment of a composition according to the disclosure.

FIG. 4 shows the results of a skin sensitivity questionnaire with subjects exposed to an embodiment of a composition according to the disclosure.

FIG. 5 shows the results of a TEWL/Hydration assay on subjects using yet another embodiment of a composition according to the disclosure.

FIG. 6 shows the effect of personal care composition (shown as TREAT) on NADPH-oxidase activity. NE-NTC: non-exposed, nontreated cells; DPI: Diphenyleneiodonium, standard NADPH oxidase inhibitor; PE-NTC: pollution-exposed & non-treated cells; PE+IC94214 0.005%: pollution-exposed & treated. ***: p<0.001; ##: p<0.01, #: p<0.05. Bars are standard deviation.

FIG. 7 is a representation of the mitochondrial membrane potential of primary human fibroblast originating from a mature donor in non-exposed & non-treated cells (NE-NTC) and in cells exposed to standardized urban pollutants (PE-NTC) with and without treatment with (personal care composition). CCCP treatment and pollution exposure were compared to the NE-NTC. Pollutant exposed cells treated with the personal care composition were compared to PE-NTC. ***: p<0.001; **: p<0.01, *: p<0.05, #: p<0.05.

FIG. 8A shows the parameters measured in mitochondrial fusion-fission assays.

FIG. 8B shows the results of mitochondrial fusion experiments. Pollution caused a decrease in long mitochondria and mitochondrial fusion/hyperfusion. Treatment with the instant personal care products (shown as PE-TREAT) restored mitochondrial fusion.

FIG. 8C shows the results of mitochondrial fission experiments. Pollution caused an increase in mitochondrial fission/hyperfission. Treatment with the instant personal care products (shown as PE-TREAT) restored mitochondrial fission.

FIG. 9 shows the results of mitochondrial network analysis. Normal cells had healthy, branched mitochondrial networks (left panel). Pollution caused collapse and disintegration of these networks (middle panel). Treatment with the personal care composition restored the healthy mitochondrial network structure (right panel).

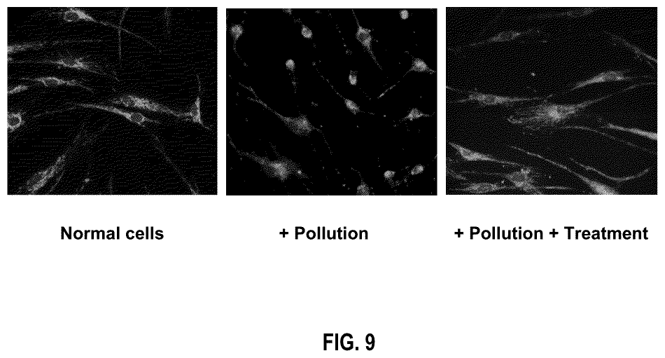

FIG. 10 shows the quantification of ER-mitochondria interactions at MAMs on non-exposed & non-treated cells (NE-NTC) and in cells exposed to standardized urban pollutants (PE-NTC) with and without treatment with personal care composition (PE-TREAT).

FIG. 11 shows the number of viable cells per fields in different experimental conditions. Error bars represent standard error of the mean.

FIG. 12 shows the effect of niacinamide (high and low concentrations) and a personal care composition (shown as Composition B, diluted to contain niacinamide concentration equal to the “low” niacinamide concentration) on NADPH-oxidase activity. NE-NTC: non-exposed, nontreated cells; PE-NTC: pollution-exposed & non-treated cells; PE+IC94214 0.005%: pollution-exposed & treated. *: p<0.05; #: p<0.05, ###: p<0.0001. Bars are standard deviation.

FIG. 13 shows the quantification of ER-mitochondria interactions at MAMs on non-exposed & non-treated cells (NE-NTC) and in cells exposed to standardized urban pollutants (PE-NTC) with and without treatment with niacinamide (“low” concentration) and a personal care composition according to the present disclosure (Composition B, diluted to contain niacinamide concentration equal to the “low” niacinamide concentration). ***: p<0.0001; ##: P<0.01. Bars are standard deviation.

### DETAILED DESCRIPTION

### Definitions

All percentages and ratios used herein are by weight of the total composition, unless otherwise designated. All measurements are understood to be made at ambient conditions, where “ambient conditions” means conditions at about 25° C., under about one atmosphere of pressure, and at about 50% relative humidity, unless otherwise designated. All numeric ranges are inclusive of narrower ranges; delineated upper and lower range limits are combinable to create further ranges not explicitly delineated.

The compositions of the present disclosure can comprise, consist essentially of, or consist of, the components described herein. As used herein, “consisting essentially of” means that the composition or component may include additional ingredients, but only if the additional ingredients do not materially alter the basic and novel characteristics of the claimed compositions or methods.

As used herein, the term about refers to ±10% of a given value.

The term “apply” or “application,” as used in reference to a composition, means to apply or spread the compositions of the present disclosure onto keratinous tissue such as the epidermis.

The term “derivatives” includes an ester, ether, amide, hydroxy, and/or salt structural analogue of a compound of interest.

The term “dermatologically acceptable” means that the compositions or components described are suitable for use in contact with human skin tissue without undue toxicity, incompatibility, instability, allergic response, and the like.

The term “keratinous tissue” refers to keratin-containing layers disposed as the outermost protective covering of mammals (e.g., humans, dogs, cats, etc.) which includes, but is not limited to, skin, lips, hair, toenails, fingernails, cuticles, hooves, etc.

As used herein, the term “pH balancing” agent refers to any compound (e.g., an acid or a base) useful for adjusting the pH of a composition to a specific value (e.g., 7, 8, 9, etc) or a specific range (e.g., pH=about 5 to about 7, about 7 to about 9, and the like). In some embodiments, the pH balancing agent is added to the composition in an amount effective to adjust the pH level of the composition to about 7 or greater, for example, about 7, or about 8, but no more than about 8.5.

In some embodiments, the pH balancing agent is added to the composition in an amount effective to adjust the pH level of the composition to about 7 or less, for example, about 7, about 6, about 5, or about 4. In some embodiments, the pH balancing agent is added to the composition in an amount effective to adjust the pH level of the composition to about 5 to about 7.

Exemplary pH balancing agents include, but are not limited to, ammonium aluminum sulfate, ammonium bicarbonate, ammonium carbonate, ammonium citrate dibasic, ammonium citrate monobasic, ammonium hydroxide, ammonium phosphate dibasic, ammonium phosphate monobasic, calcium acetate, calcium acid pyrophosphate, calcium carbonate, calcium chloride, calcium citrate, calcium fumarate, calcium Gluconate, calcium Hydroxide, calcium Lactate, calcium oxide, calcium phosphate dibasic, calcium phosphate monobasic, calcium phosphate tribasic, calcium sulfate, cream of tartar, glucono-delta-lactone, magnesium carbonate, magnesium citrate, magnesium fumarate, magnesium hydroxide, magnesium oxide, magnesium phosphate, magnesium sulfate, manganese sulfate, potassium acid tartrate, potassium aluminum sulfate, potassium bicarbonate, potassium carbonate, potassium chloride, potassium citrate, potassium fumarate, potassium hydroxide, potassium lactate, potassium phosphate dibasic, potassium phosphate tribasic, potassium sulfate, potassium tartrate, potassium tripolyphosphate, sodium acetate, sodium acid pyrophosphate, sodium acid tartrate, sodium aluminum phosphate, sodium aluminum sulfate, sodium bicarbonate, sodium bisulphate, sodium carbonate, sodium citrate, sodium fumarate, sodium gluconate, sodium hexametaphosphate, sodium hydroxide, sodium lactate, sodium phosphate dibasic, sodium phosphate monobasic, sodium phosphate tribasic, sodium potassium hexametaphosphate, sodium potassium tartrate, sodium potassium tripolyphosphate, sodium pyrophosphate tetrabasic, and sodium tripolyphosphate.

As used herein, the term “pollution” refers to polluted environment comprising pollutants, such as metals (e.g., lead), harmful chemicals (e.g., polyaromatic hydrocarbons (PAHs), nitro-PAHs, Polychlorinated Biphenyls (PCBs), chlorinated pesticide, ozone, Sulphur dioxide, carbon monoxide, nitrogen dioxide, etc.), dust and particulate matter of micrometric diameter (e.g., particulate matter of around 10 micrometers (“PM 10”), or particulate matter of around 2.5 micrometers (“PM 2.5”). In some embodiments, the term “pollution” includes polluted air (“air pollution”) or water (“water pollution”).

In some embodiments, air pollution is measured by Air Quality Index (AQI) as used in China. In some embodiments, the AQI is above 100 (“Lightly Polluted”), above 150 (“Moderately Polluted”), above 200 (“Heavily Polluted”) or above 300 (“Severely Polluted”). In some embodiments, AQI level is based on the level of six atmospheric pollutants, namely sulfur dioxide (SO₂), nitrogen dioxide (NO₂), suspended particulates smaller than 10 μm in aerodynamic diameter (PM 10), suspended particulates smaller than 2.5 pin in aerodynamic diameter (PM 2,5), carbon monoxide (CO), and ozone (O₃).

The term “safe and effective amount” means an amount of a compound or composition sufficient to significantly induce a positive benefit.

The term “sensitive skin” refers to a neurosensory skin condition that results in disparate clinical signs, including irritation, erythema or dry skin. More precisely, sensitive skin is a syndrome defined by the occurrence of unpleasant sensations, such as stings, burns, pain, pruritus or tingling, in response to stimuli that should not normally cause such sensations; in addition, sensitive skin is characterized by the absence of a link between these sensations and any skin pathology (Misery et al., Acta Derm Venereol 2017, 97: 4-6). Suitable assays for measuring sensitivity, inflammation, irritation of the skin are known in the art. One such assay is the Jordan-King assay, as set forth in Jordan, W. P. 1994, Jordan/King modification of the Draize Repeat Insult Patch Test, Clairol Study #94046, Test Dates Oct. 3, 1994-Nov. 11, 1994, the entire contents of which are hereby incorporated by reference.

As used herein, the term “signs of aging of skin” includes wrinkles, dryness, loss of elasticity, suppleness, firmness, luminosity, and increase of sensitivity in the skin.

The term “leave-on,” in reference to personal care compositions, means a composition intended to be applied to and allowed to remain on the keratinous tissue. These leave-on compositions are to be distinguished from compositions which are applied to the skin and subsequently (in a few minutes or less) removed either by washing, rinsing, wiping, or the like. Leave-on compositions exclude rinse-off applications such as shampoos, facial cleansers, hand cleansers, body wash, or body cleansers. The leave-on compositions may be substantially free of cleansing or detersive surfactants. For example, “leave-on compositions” may be left on the keratinous tissue for at least 15 minutes. For example, leave-on compositions may comprise less than 1% detersive surfactants, less than 0.5% detersive surfactants, or 0% detersive surfactants. The compositions may, however, contain emulsifying or other processing surfactants that are not intended to provide any significant cleansing benefits when applied topically to the skin.

The term “rinse-off” means the intended personal care composition usage includes application to the keratinous tissue followed by rinsing and/or wiping the product from the keratinous tissue within a few seconds to minutes of the application step. The product is generally applied and rinsed in the same usage event, for example, a shower.

The term “soluble” means at least about 0.1 g of solute dissolves in 100 ml of solvent, at 25° C. and 1 atm of pressure.

As used herein, the term “personal care composition” refers to compositions suitable for topical application on mammalian keratinous tissue.

### Personal Care Compositions

The instant disclosure provides personal care compositions that slow down aging or reduce the signs of aging, improve radiance and reduce wrinkles in sensitive skin. Without committing to one particular theory, the personal care compositions of the instant disclosure slow down aging and reduce the signs of aging in sensitive skin by improving collagen production, regulating epidermis cell metabolism, and reducing inflammation and oxidative stress. The personal care compositions of the instant disclosure strengthen skin barrier, deeply hydrate the upper layers of the skin, protect and sooth the most sensitive skin, and revitalize the skin to a healthier youthful appearance. The personal care compositions of the instant disclosure are suitable for use on sensitive skin and shows effects as early as within 10-14 days of daily use.

An aspect of the disclosure is directed to a personal care composition comprising, by weight: (a) 0.1%-10% (e.g., 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.7%, 2.8%, 2.9%, 3%, 3.1%, 3.2%, 3.3%, 3.4%, 3.5%, 3.6%, 3.7%, 3.8%, 3.9%, 4%, 4.1%, 4.2%, 4.3%, 4.4%, 4.5%, 4.6%, 4.7%, 4.8%, 4.9%, 5%, 5.1%, 5.2%, 5.3%, 5.4%, 5.5%, 5.6%, 5.7%, 5.8%, 5.9%, 6%, 6.1%, 6.2%, 6.3%, 6.4%, 6.5%, 6.6%, 6.7%, 6.8%, 6.9%, 7%, 7.1%, 7.2%, 7.3%, 7.4%, 7.5%, 7.6%, 7.7%, 7.8%, 7.9%, 8%, 8.1%, 8.2%, 8.3%, 8.4%, 8.5%, 8.6%, 8.7%, 8.8%, 8.9%, 9%, 9.1%, 9.2%, 9.3%, 9.4%, 9.5%, 9.6%, 9.7%, 9.8%, 9.9%, 10%) niacinamide, (b) 0.0001%-10% (e.g., 0.0001%, 0.0002%, 0.0003%, 0.0004%, 0.0005%, 0.0006%, 0.0007%, 0.0008%, 0.0009%, 0.001%, 0.002%, 0.003%, 0.004%, 0.005%, 0.006%, 0.007%, 0.008%, 0.009%, 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.7%, 2.8%, 2.9%, 3%, 3.1%, 3.2%, 3.3%, 3.4%, 3.5%, 3.6%, 3.7%, 3.8%, 3.9%, 4%, 4.1%, 4.2%, 4.3%, 4.4%, 4.5%, 4.6%, 4.7%, 4.8%, 4.9%, 5%, 5.1%, 5.2%, 5.3%, 5.4%, 5.5%, 5.6%, 5.7%, 5.8%, 5.9%, 6%, 6.1%, 6.2%, 6.3%, 6.4%, 6.5%, 6.6%, 6.7%, 6.8%, 6.9%, 7%, 7.1%, 7.2%, 7.3%, 7.4%, 7.5%, 7.6%, 7.7%, 7.8%, 7.9%, 8%, 8.1%, 8.2%, 8.3%, 8.4%, 8.5%, 8.6%, 8.7%, 8.8%, 8.9%, 9%, 9.1%, 9.2%, 9.3%, 9.4%, 9.5%, 9.6%, 9.7%, 9.8%, 9.9%, 10%) Edelweiss (Leontopodium alpinum) extract, (c) 0.001%-10% (e.g., 0.001%, 0.002%, 0.003%, 0.004%, 0.005%, 0.006%, 0.007%, 0.008%, 0.009%, 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.7%, 2.8%, 2.9%, 3%, 3.1%, 3.2%, 3.3%, 3.4%, 3.5%, 3.6%, 3.7%, 3.8%, 3.9%, 4%, 4.1%, 4.2%, 4.3%, 4.4%, 4.5%, 4.6%, 4.7%, 4.8%, 4.9%, 5%, 5.1%, 5.2%, 5.3%, 5.4%, 5.5%, 5.6%, 5.7%, 5.8%, 5.9%, 6%, 6.1%, 6.2%, 6.3%, 6.4%, 6.5%, 6.6%, 6.7%, 6.8%, 6.9%, 7%, 7.1%, 7.2%, 7.3%, 7.4%, 7.5%, 7.6%, 7.7%, 7.8%, 7.9%, 8%, 8.1%, 8.2%, 8.3%, 8.4%, 8.5%, 8.6%, 8.7%, 8.8%, 8.9%, 9%, 9.1%, 9.2%, 9.3%, 9.4%, 9.5%, 9.6%, 9.7%, 9.8%, 9.9%, 10%) rice (Oryza sativa) lees concentrate, (d) 0.1%-10% (e.g., 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.7%, 2.8%, 2.9%, 3%, 3.1%, 3.2%, 3.3%, 3.4%, 3.5%, 3.6%, 3.7%, 3.8%, 3.9%, 4%, 4.1%, 4.2%, 4.3%, 4.4%, 4.5%, 4.6%, 4.7%, 4.8%, 4.9%, 5%, 5.1%, 5.2%, 5.3%, 5.4%, 5.5%, 5.6%, 5.7%, 5.8%, 5.9%, 6%, 6.1%, 6.2%, 6.3%, 6.4%, 6.5%, 6.6%, 6.7%, 6.8%, 6.9%, 7%, 7.1%, 7.2%, 7.3%, 7.4%, 7.5%, 7.6%, 7.7%, 7.8%, 7.9%, 8%, 8.1%, 8.2%, 8.3%, 8.4%, 8.5%, 8.6%, 8.7%, 8.8%, 8.9%, 9%, 9.1%, 9.2%, 9.3%, 9.4%, 9.5%, 9.6%, 9.7%, 9.8%, 9.9%, 10%) panthenol, and (e) 0.001%-5% (e.g., 0.001%, 0.002%, 0.003%, 0.004%, 0.005%, 0.006%, 0.007%, 0.008%, 0.009%, 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.7%, 2.8%, 2.9%, 3%, 3.1%, 3.2%, 3.3%, 3.4%, 3.5%, 3.6%, 3.7%, 3.8%, 3.9%, 4%, 4.1%, 4.2%, 4.3%, 4.4%, 4.5%, 4.6%, 4.7%, 4.8%, 4.9%, 5%) soy-derived glycopeptides.

Another aspect of the disclosure is directed to a personal care composition comprising, by weight: (a) 0.1%-4% (e.g., 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.7%, 2.8%, 2.9%, 3%, 3.1%, 3.2%, 3.3%, 3.4%, 3.5%, 3.6%, 3.7%, 3.8%, 3.9%, 4%,) niacinamide, (b) 0.0001%-4% (e.g., 0.0001%, 0.0002%, 0.0003%, 0.0004%, 0.0005%, 0.0006%, 0.0007%, 0.0008%, 0.0009%, 0.001%, 0.002%, 0.003%, 0.004%, 0.005%, 0.006%, 0.007%, 0.008%, 0.009%, 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.7%, 2.8%, 2.9%, 3%, 3.1%, 3.2%, 3.3%, 3.4%, 3.5%, 3.6%, 3.7%, 3.8%, 3.9%, 4%) Edelweiss (Leontopodium alpinum) extract, (c) 0.001%-2% (e.g., 0.001%, 0.002%, 0.003%, 0.004%, 0.005%, 0.006%, 0.007%, 0.008%, 0.009%, 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%) rice (Oryza sativa) lees concentrate, (d) 0.1%-3% (e.g., 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.7%, 2.8%, 2.9%, 3%) panthenol, and (e) 0.002%-4% (e.g., 0.002%, 0.003%, 0.004%, 0.005%, 0.006%, 0.007%, 0.008%, 0.009%, 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.7%, 2.8%, 2.9%, 3%, 3.1%, 3.2%, 3.3%, 3.4%, 3.5%, 3.6%, 3.7%, 3.8%, 3.9%, 4%) soy-derived glycopeptides.

Another aspect of the disclosure is directed to a personal care composition comprising, by weight: (a) 0.1%-10% (e.g., 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.7%, 2.8%, 2.9%, 3%, 3.1%, 3.2%, 3.3%, 3.4%, 3.5%, 3.6%, 3.7%, 3.8%, 3.9%, 4%, 4.1%, 4.2%, 4.3%, 4.4%, 4.5%, 4.6%, 4.7%, 4.8%, 4.9%, 5%, 5.1%, 5.2%, 5.3%, 5.4%, 5.5%, 5.6%, 5.7%, 5.8%, 5.9%, 6%, 6.1%, 6.2%, 6.3%, 6.4%, 6.5%, 6.6%, 6.7%, 6.8%, 6.9%, 7%, 7.1%, 7.2%, 7.3%, 7.4%, 7.5%, 7.6%, 7.7%, 7.8%, 7.9%, 8%, 8.1%, 8.2%, 8.3%, 8.4%, 8.5%, 8.6%, 8.7%, 8.8%, 8.9%, 9%, 9.1%, 9.2%, 9.3%, 9.4%, 9.5%, 9.6%, 9.7%, 9.8%, 9.9%, 10%) niacinamide, (b) 0.0001%-10% (e.g., 0.0001%, 0.0002%, 0.0003%, 0.0004%, 0.0005%, 0.0006%, 0.0007%, 0.0008%, 0.0009%, 0.001%, 0.002%, 0.003%, 0.004%, 0.005%, 0.006%, 0.007%, 0.008%, 0.009%, 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.7%, 2.8%, 2.9%, 3%, 3.1%, 3.2%, 3.3%, 3.4%, 3.5%, 3.6%, 3.7%, 3.8%, 3.9%, 4%, 4.1%, 4.2%, 4.3%, 4.4%, 4.5%, 4.6%, 4.7%, 4.8%, 4.9%, 5%, 5.1%, 5.2%, 5.3%, 5.4%, 5.5%, 5.6%, 5.7%, 5.8%, 5.9%, 6%, 6.1%, 6.2%, 6.3%, 6.4%, 6.5%, 6.6%, 6.7%, 6.8%, 6.9%, 7%, 7.1%, 7.2%, 7.3%, 7.4%, 7.5%, 7.6%, 7.7%, 7.8%, 7.9%, 8%, 8.1%, 8.2%, 8.3%, 8.4%, 8.5%, 8.6%, 8.7%, 8.8%, 8.9%, 9%, 9.1%, 9.2%, 9.3%, 9.4%, 9.5%, 9.6%, 9.7%, 9.8%, 9.9%, 10%) Edelweiss (Leontopodium alpinum) extract, (c) 0.001%-10% (e.g., 0.001%, 0.002%, 0.003%, 0.004%, 0.005%, 0.006%, 0.007%, 0.008%, 0.009%, 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.7%, 2.8%, 2.9%, 3%, 3.1%, 3.2%, 3.3%, 3.4%, 3.5%, 3.6%, 3.7%, 3.8%, 3.9%, 4%, 4.1%, 4.2%, 4.3%, 4.4%, 4.5%, 4.6%, 4.7%, 4.8%, 4.9%, 5%, 5.1%, 5.2%, 5.3%, 5.4%, 5.5%, 5.6%, 5.7%, 5.8%, 5.9%, 6%, 6.1%, 6.2%, 6.3%, 6.4%, 6.5%, 6.6%, 6.7%, 6.8%, 6.9%, 7%, 7.1%, 7.2%, 7.3%, 7.4%, 7.5%, 7.6%, 7.7%, 7.8%, 7.9%, 8%, 8.1%, 8.2%, 8.3%, 8.4%, 8.5%, 8.6%, 8.7%, 8.8%, 8.9%, 9%, 9.1%, 9.2%, 9.3%, 9.4%, 9.5%, 9.6%, 9.7%, 9.8%, 9.9%, 10%) rice (Oryza sativa) lees concentrate, and (d) 0.1%-10% (e.g., 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.7%, 2.8%, 2.9%, 3%, 3.1%, 3.2%, 3.3%, 3.4%, 3.5%, 3.6%, 3.7%, 3.8%, 3.9%, 4%, 4.1%, 4.2%, 4.3%, 4.4%, 4.5%, 4.6%, 4.7%, 4.8%, 4.9%, 5%, 5.1%, 5.2%, 5.3%, 5.4%, 5.5%, 5.6%, 5.7%, 5.8%, 5.9%, 6%, 6.1%, 6.2%, 6.3%, 6.4%, 6.5%, 6.6%, 6.7%, 6.8%, 6.9%, 7%, 7.1%, 7.2%, 7.3%, 7.4%, 7.5%, 7.6%, 7.7%, 7.8%, 7.9%, 8%, 8.1%, 8.2%, 8.3%, 8.4%, 8.5%, 8.6%, 8.7%, 8.8%, 8.9%, 9%, 9.1%, 9.2%, 9.3%, 9.4%, 9.5%, 9.6%, 9.7%, 9.8%, 9.9%, 10%) panthenol.

In some embodiments, “niacinamide” comprises the formula:

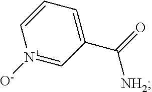

derivatives thereof; and salts of any of the foregoing.

In some embodiments, the personal care composition comprises 0.1%-2% (e.g., 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%) niacinamide by weight. In some embodiments, the personal care composition comprises 0.5%-1.5% (e.g., 0.5%, 0.6%, 0.7%, 0.8%, 0.9%, 1%, 1.1%, 1.2%, 1.3%, 1.4%, 1.5%) niacinamide by weight.

In some embodiments, the Edelweiss extract is as described in WO2001/087256 and EP 2623094. In some embodiments, the Edelweiss extract is the product sold under the trade name Alpaflor® EDELWEISS by DSM Nutritional Products. In some embodiments, the Edelweiss (Leontopodium alpinum) extract comprises Chlorogenic acid, Luteoline-4′-O-glucoside, Apigenine-7-O-glucoside, luteolin, and Leontopodic Acid as bioactive agents. In some embodiments, the Edelweiss extract contains tannin. In some embodiments, the Edelweiss extract is blended with other agents such as propanetriol, glycerin, water, citric acid, sodium benzoate, ethyl alcohol, and potassium sorbate. In some embodiments, the personal care composition comprises 0.1%-2% (e.g., 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%) Edelweiss (Leontopodium alpinum) extract by weight. In some embodiments, the personal care composition comprises 0.5%-1.5% (e.g., 0.5%, 0.6%, 0.7%, 0.8%, 0.9%, 1%, 1.1%, 1.2%, 1.3%, 1.4%, 1.5%) Edelweiss (Leontopodium alpinum) extract by weight. In some embodiments, the personal care composition comprises 0.0001%-0.5% (e.g., 0.0001%, 0.0002%, 0.0003%, 0.0004%, 0.0005%, 0.0006%, 0.0007%, 0.0008%, 0.0009%, 0.001%, 0.002%, 0.003%, 0.004%, 0.005%, 0.006%, 0.007%, 0.008%, 0.009%, 0.01%, 0.02%, 0.03%, 0.04%, 0.05%, 0.06%, 0.07%, 0.08%, 0.09%, 0.1%, 0.2%, 0.3%, 0.4%, or 0.5%) Edelweiss (Leontopodium alpinum) extract by weight.

In some embodiments, the Edelweiss (Leontopodium alpinum) extract is provided (e.g., from a manufacturer) in a solution comprising the Leontopodium alpinum extract and a solvent (e.g., water, glycerin, or combinations thereof) with other ingredients (e.g., citric acid, sodium benzoate, potassium sorbate, etc.), which solution is subsequently added to the personal care composition of the present disclosure. In some embodiments, the concentration of Leontopodium alpinum extract in the provided solution (e.g., obtained from the manufacturer) is less than or equal to 10 wt. %, 9.5 wt. %, 9 wt. %, 8.5 wt. %, 8 wt. %, 7.5 wt. %, 7 wt. %, 6.5 wt. %, 6 wt. %, 5.5 wt. %, 5 wt. %, 4.5 wt. %, 4 wt. %, 3.5 wt. %, 3 wt. %, 2.9 wt. %, 2.8 wt. %, 2.7 wt. %, 2.6 wt. %, 2.5 wt. %, 2.4 wt. %, 2.3 wt. %, 2.2 wt. %, 2.1 wt. %, 2.0 wt. %, 1.9 wt. %, 1.8 wt. %, 1.7 wt. %, 1.6 wt. %, 1.5 wt. %, 1.4 wt. %, 1.3 wt. %, 1.2 wt. %, 1.0 wt. %, 0.5 wt. %, 0.1 wt. %, or less, relative to the weight of the provided solution. Thus in some embodiments, the concentration of Edelweiss (Leontopodium alpinum) extract in the personal care composition (as set forth herein) indicates a concentration of a solution comprising the Edelweiss (Leontopodium alpinum) extract, relative to the weight of the personal care composition. For instance, a concentration of 0.0001%-10% Edelweiss (Leontopodium alpinum) extract may indicate a concentration of the solution comprising Edelweiss (Leontopodium alpinum) extract, relative to the total weight of the personal care composition.

Alternatively, in some embodiments, a concentration of 0.0001%-5% Edelweiss (Leontopodium alpinum) extract (or any other concentration set forth herein) may indicate a concentration of the “dry” Edelweiss (Leontopodium alpinum) extract botanical active, relative to the total weight of the personal care composition. By way of non-limiting example, if the Edelweiss (Leontopodium alpinum) extract is provided in a solution that is added at, e.g., 3.0 wt. %, relative to the total weight of the personal care composition, and the “dry weight” of the soy-derived glycopeptides in the provided solution is 8.5 wt. %, relative to the total weight of the provided solution, then the “dry weight” of the soy-derived glycopeptides in the personal care composition would be 0.255 wt. %, relative to the total weight of the personal care composition.

In some embodiments, the rice (Oryza sativa) lees concentrate comprises amino acids, oleic acid, linolenic acid, phytic acid, palmitic acid and vitamin B derivatives as bioactive agents. In some embodiments, the rice (Oryza sativa) lees concentrate comprises water, glycerin, Oryza sativa lees extract, propanediol, 1,2, hexanediol and ethylhexylglycerin. In some embodiments, the rice (Oryza sativa) lees concentrate has a pH between 4.0 and 7.0. In some embodiments, the rice (Oryza sativa) lees concentrate has a specific gravity (d20/20) between 0.99 and 1.05.

In some embodiments, the personal care composition comprises 0.1%-2% (e.g., 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%) rice (Oryza sativa) lees concentrate by weight. In some embodiments, the personal care composition comprises 0.5%-1.5% (e.g., 0.5%, 0.6%, 0.7%, 0.8%, 0.9%, 1%, 1.1%, 1.2%, 1.3%, 1.4%, 1.5%) rice (Oryza sativa) lees concentrate by weight. In some embodiments, the personal care composition comprises 0.001%-1% (e.g., 0.001%, 0.002%, 0.003%, 0.004%, 0.005%, 0.006%, 0.007%, 0.008%, 0.009%, 0.1%, 0.2%, 0.3%, 0.4%, 0.5%, 0.6%, 0.7%, 0.8%, 0.9%, 1%) rice (Oryza sativa) lees concentrate by weight.

Similar to Edelweiss (Leontopodium alpinum) extract, the concentration of rice (Oryza sativa) lees concentrate may be a concentration, relative to the total weight of the personal care composition, of a provided solution (e.g., from a manufacturer) comprising the active ingredient of rice (Oryza sativa) lees concentrate and one or more solvents (e.g., water, glycerin, propanediol, 1,2-hexanediol, ethylhexylglycerin, or combinations thereof). Alternatively, the concentration of rice (Oryza sativa) lees concentrate may be a “dry weight” concentration, relative to the total weight of the personal care composition, of the active ingredient of rice (Oryza sativa) lees concentrate. For instance, if the provided solution comprising rice (Oryza sativa) lees concentrate is present in the personal care composition at a concentration of 1.0 wt. %, and the active ingredient of rice (Oryza sativa) lees concentrate is present in the provided solution at a concentration of 10 wt. %, relative to the total weight of the provided solution, then the overall “dry weight” of the active ingredient in rice (Oryza sativa) lees concentrate would be 0.1 wt. %, relative to the total weight of the personal care composition.

In some embodiments, the personal care composition further comprises 0.01%-2% (e.g., 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, or 2%) adenosine by weight of the total composition.

In some embodiments, the personal care composition further comprises 0.0001%-3% (e.g., 0.0001%, 0.0002%, 0.0003%, 0.0004%, 0.0005%, 0.0006%, 0.0007%, 0.0008%, 0.0009%, 0.001%, 0.002%, 0.003%, 0.004%, 0.005%, 0.006%, 0.007%, 0.008%, 0.009%, 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.7%, 2.8%, 2.9%, or 3%) Buddleja davidii extract by weight of the total composition.

In some embodiments, the personal care composition further comprises 0.0001%-3% (e.g., 0.0001%, 0.0002%, 0.0003%, 0.0004%, 0.0005%, 0.0006%, 0.0007%, 0.0008%, 0.0009%, 0.001%, 0.002%, 0.003%, 0.004%, 0.005%, 0.006%, 0.007%, 0.008%, 0.009%, 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.7%, 2.8%, 2.9%, or 3%) Thymus vulgaris extract by weight of the total composition.

Similar to Edelweiss (Leontopodium alpinum) extract and rice (Oryza sativa) lees concentrate, the reported concentration of Thymus vulgaris extract or Buddleja davidii extract may be the concentration of a provided solution comprising these actives or the “dry weight” of these actives, relative to the total weight of the personal care composition.

In some embodiments, “panthenol” comprises the formula:

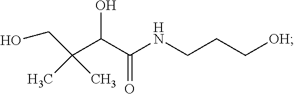

derivatives thereof; and salts of any of the foregoing.

In some embodiments, the personal care composition comprises 0.1%-2% (e.g., 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%) panthenol by weight. In some embodiments, the personal care composition comprises 0.5%-1.5% (e.g., 0.5%, 0.6%, 0.7%, 0.8%, 0.9%, 1%, 1.1%, 1.2%, 1.3%, 1.4%, 1.5%) panthenol by weight.

In some embodiments, the soy-derived glycopeptides are glycopeptides that are derived from highly purified soya fiber. In some embodiments, the soy-derived glycopeptides may be blended with stabilizers and preservatives. An exemplary soy-derived glycopeptide is sold under the trade name PRO-COLL-ONE+® by Silab.

In some embodiments, the soy-derived glycopeptides are provided (e.g., from a manufacturer) in a solution comprising the soy-derived glycopeptides (e.g., hydrolyzed soy flour) and a solvent (e.g., water, caprylyl glycol, 1,2-hexanediol, or combinations thereof), which solution is subsequently added to the personal care composition of the present disclosure. In some embodiments, the concentration of soy-derived glycopeptides in the provided solution (e.g., obtained from the manufacturer) is less than or equal to 10 wt. %, 9 wt. %, 8 wt. %, 7 wt. %, 6 wt. %, 5 wt. %, 4 wt. %, 3 wt. %, 2.9 wt. %, 2.8 wt. %, 2.7 wt. %, 2.6 wt. %, 2.5 wt. %, 2.4 wt. %, 2.3 wt. %, 2.2 wt. %, 2.1 wt. %, 2.0 wt. %, 1.9 wt. %, 1.8 wt. %, 1.7 wt. %, 1.6 wt. %, 1.5 wt. %, 1.4 wt. %, 1.3 wt. %, 1.2 wt. %, 1.0 wt. %, 0.5 wt. %, 0.1 wt. %, or less, relative to the weight of the provided solution. Thus in some embodiments, the concentration of soy-derived glycopeptides (as set forth herein) indicates a concentration of a solution comprising the soy-derived glycopeptides (e.g., hydrolyzed soy flour), relative to the weight of the personal care composition. For instance, a concentration of 0.001%-5% soy-derived glycopeptides may indicate a concentration of the solution comprising soy-derived glycopeptides, relative to the total weight of the personal care composition.

Alternatively, in some embodiments, a concentration of 0.001%-5% soy-derived glycopeptides may indicate a concentration of the “dry” soy-derived glycopeptides (e.g., hydrolyzed soy flour) per se, relative to the total weight of the personal care composition. By way of non-limiting example, if the soy-derived glycopeptides (e.g., hydrolyzed soy flour) are provided in a solution that is added at 2.0 wt. %, relative to the total weight of the personal care composition, and the “dry weight” of the soy-derived glycopeptides in the provided solution is 3.0 wt. %, relative to the total weight of the provided solution, then the “dry weight” of the soy-derived glycopeptides in the personal care composition would be 0.06 wt. %, relative to the total weight of the personal care composition.

In some embodiments, the personal care composition does not comprise any retinoid or retinoid derivatives.

In some embodiments, the personal care composition further comprises at least one of the following: water, anti-dehydration agent, light protective filter, antioxidant, preservative, gelling agent, solvent, pH balancing agent, and surfactant.

In some embodiments, the anti-dehydration agent comprises at least one of xylitylglucoside, anhydroxylitol, and xylitol. In some embodiments, the anti-dehydration agent comprises each of xylitylglucoside, anhydroxylitol, and xylitol. In some embodiments, the anti-dehydration agent makes up, by weight, 0.01-0.5% of the personal care composition.

In some embodiments, the light protective filter comprises at least one of zinc oxide, titanium dioxide and iron oxide.

In some embodiments, the personal care composition further comprises at least one of the following: hydrolyzed hyaluronic acid, glycerin, sodium hydroxide, phenoxyethanol, xantham gum, sodium polyacryloyldimethyl taurate, undecane, tridecane, tocopherol, dimethicone, titanium dioxide, tin oxide, and calcium aluminum borosilicate. In some embodiments, the personal care composition comprises each of the following: hydrolyzed hyaluronic acid, glycerin, sodium hydroxide, phenoxyethanol, xantham gum, sodium polyacryloyldimethyl taurate, undecane, tridecane, tocopherol, dimethicone, titanium dioxide, tin oxide, and calcium aluminum borosilicate.

In some embodiments, the personal care composition comprises at least one of the following: hydrolyzed hyaluronic acid (0.001%-0.05% by weight), glycerin (0.1%-7% by weight), sodium hydroxide (0.001%-0.1% by weight), phenoxyethanol (0.1%-1% by weight), xantham gum (0.05%-0.5% by weight), sodium polyacryloyldimethyl taurate (0.1%-1.2% by weight), an emollient composition comprising undecane, tridecane, and tocopherol (0.05%-0.5% by weight), dimethicone (0.5%-5% by weight), a pigment composition comprising titanium dioxide, tin oxide, and calcium aluminum borosilicate (0.005%-0.05% by weight).

In some embodiments, the emollient composition comprises Cetiol Ultimate™.

In some embodiments, the pigment composition comprises Syncrystal Gold™.

In some embodiments, the personal care composition further comprises an exfoliator. In some embodiments, the exfoliator is jojoba beads containing vitamin E.

In some embodiments, the personal care composition is a cream, a lotion, a gel, a mask, or a spray. The personal care composition of the disclosure may be a skin care, anti-perspirant, deodorant, cosmetic, or hair care product. The personal care composition may be used as, for example, a moisturizer, conditioner, anti-aging compound, skin lightener, skin mask, sunscreen, sunless tanner, shave preparation, after-shave, and combinations thereof. In certain embodiments, the composition is applied to the face, neck, hands, arms, and other typically exposed areas of the body.

The personal care composition may involve a wide variety of forms. Non-limiting examples include simple solutions (e.g., water or oil-based), dispersions, and emulsions. The personal care compositions may be fluid or solid (gels, sticks, flowable solids, amorphous materials). In certain embodiments, the personal care composition is in the form of an emulsion. Emulsion may be generally classified as having a continuous aqueous phase (e.g., oil-in-water and water-in-oil-in-water) or a continuous oil phase (e.g., water-in-oil and oil-in-water-in-oil).

In some embodiments, the personal care composition is a leave-on composition.

In some embodiments, the personal care composition is a rinse-off composition.

In certain embodiment, the personal care composition has a turbidity of from about 5 Nephelometric Turbidity Unit (NTU) to less than about 3000 NTU, 1000 NTU, 500 NTU, or 100 NTU.

In select embodiments, the personal care composition may be in a form comprising at least one discrete, visually distinct first phase and at least one discrete, visually distinct second phase. For purposes of these select embodiments, “visually distinct” means that the phases can be separately seen by the human eye as distinctly separate regions (i.e., not emulsions or dispersions of particles. In some embodiments, at least one phase forms a stable pattern, for example a continuous or discontinuous line, a spiral, a curve, or other geometric shape, within a transparent phase, where “within” means that one phase is substantially surrounded by the other phase the and does not contact the side of a container. Alternatively, the phases may form a swirled pattern, wherein both phases alternately contact the side of a container and wherein the width of each of phase, when viewed through the side of a transparent container, is substantially constant, but may differ from each other. Alternatively, the phases may form a marbled pattern, wherein the phases alternately contact the side of the container and wherein the width of the individual phases, when viewed through the side of a transparent container, may vary throughout the composition. In one embodiment, the first phase is a transparent, clear or translucent aqueous phase and the second phase is either an opaque white or colored non-aqueous phase. In another alternative embodiment, at least one aqueous phase forms a pattern within a non-aqueous phase. In some embodiments, the instant personal care compositions may comprise a three or more visually distinct and stable phases. Discrete, visually distinct multi-phase compositions are described in U.S. Patent Application Publication Nos. 2007/0297996, 2004/0057920, and 2004/0219119, which are all incorporated by reference in their entireties.

### Carriers

The personal care composition may comprise a carrier. Carriers may be selected for various stability, aesthetics, and/or compatibility with other materials present in the personal care composition.

Suitable carriers include water and/or water-soluble solvents. The personal care composition may comprise from about 1% to about 95% by weight of water and/or water-equivalent solvent. The composition may comprise from about 1%, 3%, 5%, 10%, 15%, 20%, 25%, 30%, 35%, 40%, 45%, 50%, 55%, 60%, 65%, 70%, 75%, 80%, 85%, or 90% to about 90%, 85%, 80%, 75%, 70%, 65%, 60%, 55%, 50%, 45%, 40%, 35%, 30%, 25%, 20%, 15%, 10%, or 5% water and/or a water-equivalent solvent “Water-equivalent solvent” refers to a compound which has a similar ability as water to solubilize a material. Suitable water-equivalent solvents include monohydric alcohols, dihydric alcohols, polyhydric alcohols, glycerol, glycols, polyalkylene glycols such as polyethylene glycol, and mixtures thereof. Particularly suitable solvents, include lower aliphatic alcohols such as ethanol, propanol, butanol, isopropanol; diols such as 1,2-propanediol, 1,3-propanediol, butanediol, pentanediol, hexanediol, heptanediol, decanediol; glycerin; water, and mixtures thereof. In certain embodiments, the personal care composition comprises water, diols, glycerin, and combinations thereof.

Suitable carriers also include oils. The personal care composition may comprise from about 1% to about 95% by weight of one or more oils. The composition may comprise from about 1%, 3%, 5%, 10%, 15%, 20%, 25%, 30%, 35%, 40%, 45%, 50% 55%, 60%, 65%, 70%, 75%, 80%, 85%, or 90% to about 90%, 85%, 80%, 75%, 70%, 65%, 60%, 55%, 50%, 45%, 40%, 35%, 30%, 25%, 20%, 15%, 10%, or 5% of one or more oils. Oils may be used to solubilize, disperse, or carry materials that are not suitable for water or water-equivalent solvents. Suitable oils include silicones, hydrocarbons, esters, fatty amides, ethers, and mixtures thereof. Oils may be fluid at room temperature. However, certain personal care product forms (i.e., solid or semi-solid stick) may require non-fluid oils. The oils may be volatile or nonvolatile. “Non-volatile” means a material that exhibits a vapor pressure of no more than about 0.2 mm Hg at 25° C. at one atmosphere and/or a material that has a boiling point at one atmosphere of at least about 300° C. “Volatile” means that the material exhibits a vapor pressure of at least about 0.2 mm. of mercury at 20° C. Volatile oils may be used to provide a lighter feel when a heavy, greasy film is undesirable.

Suitable oils include volatile oils. In certain embodiments, the volatile oils may have a viscosity ranging from about 0.5 to about 5 centistokes 25° C. Volatile oils may be used to promote more rapid drying of the skin care composition after it is applied to skin. Nonvolatile oils are also suitable for use in the composition. Nonvolatile oils are often used for emolliency and protective properties. Nonvolatile oils may have a viscosity ranging from about 5 to about 800,000 cst (or greater) or from about 20 to about 200,000 cst.

Suitable silicone oils include polysiloxanes. Polylsiloxanes may have a viscosity of from about 0.5 to about 1,000,000 centistokes at 25° C. Such polysiloxanes can be represented by the general chemical formula:

R₃SiO[R₂SiO]ₓSiR₃

wherein R is independently selected from hydrogen or C₁₋₃₀ straight or branched chain, saturated or unsaturated alkyl, phenyl or aryl, trialkylsiloxy; and x is an integer from 0 to about 10,000, chosen to achieve the desired molecular. In certain embodiments, R is hydrogen, methyl, or ethyl. Commercially available polysiloxanes include the polydimethylsiloxanes, which are also known as dimethicones, examples of which include the DM Fluid series from Shin-Etsu, the Vicasil® series sold by Momentive Performance Materials Inc., and the Dow Corning® 200 series sold by Dow Corning Corporation. Specific examples of suitable polydimethylsiloxanes include Dow Corning® 200 fluids (also sold as Xiameter® PMX-200 Silicone Fluids) having viscosities of 0.65, 1.5, 50, 100, 350, 10.000, 12,500 100,000, and 300,000 centistokes.

Suitable dimethicones include those represented by the chemical formula:

R₃SiO[R₂SiO]ₓ[RR′SiO]_{y}SiR₃,

wherein R and R′ are each independently hydrogen or C₁₋₃₀ straight or branched chain, saturated or unsaturated alkyl, aryl, or trialkylsiloxy; and x and y are each integers of 1 to 1,000,000 selected to achieve the desired molecular weight. Suitable silicones include phenyl dimethicone (Botansil™ PD-151 from Botanigenics, Inc.), diphenyl dimethicone (KF-53 and KF-54 from Shin-Etsu), phenyl trimethicone (556 Cosmetic Grade Fluid from Dow Corning), or trimethylsiloxyphenyl dimethicone (PDM-20, PDM-200, or PDM-1000 from Wacker-Belsil). Other examples include alkyl dimethicones wherein at least R′ is a fatty alkyl (e.g., C₁₂₋₂₂). A suitable alkyl dimethicone is cetyl dimethicone, wherein R′ is a straight C16 chain and R is methyl. Cetyl dimethicone, is available as s 2502 Cosmetic Fluid from Dow Corning or as Abil Wax 9801 or 9814 from Evonik Goldschmidt GmbH.

Cyclic silicones are one type of silicone oil that may be used in the personal care compositions of the instant disclosure. Such silicones have the general formula:

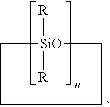

wherein R is independently selected from hydrogen or C₁₋₃₀ straight or branched chain, saturated or unsaturated alkyl, phenyl or aryl, trialkylsiloxy; and where n=3-8 and mixtures thereof. Commonly, a mixture of cyclomethicones is used where n is 4, 5, and/or 6. Commercially available cyclomethicones include Dow Corning UP-1001 Ultra Pure Fluid (i.e., n=4), Dow Corning XIAMETER® PMX-0245 (i.e., n=5), Dow Corning XIAMETER® PMX-0245 (i.e. n=6). Dow Corning 245 fluid (i.e., n=4 and 5), and Dow Corning 345 fluid (i.e., n=4, 5, and 6).

Other silicone oils suitable for use in the personal care composition include polymers having the general formula:

(R₁)ₙG₃₋ₐ-Si—(—OSiG₂)ₙ-(-OSiG_{b}(R₁)_{2-b})ₘ—O—SiG₃₋ₐ(R₁)ₐ

wherein G is hydrogen, phenyl, hydroxy, or C₁-C₈ alkyl, preferably methyl; a is a number 0-3; b is 0 or 1, preferably 1; n is a number from 0 to 1,999 (alternately, from 49 to 499); m is an integer from 1 to 2,000 (alternately, from 1 to 10); the sum of n and m is a number from 1 to 2,000 (alternately, from 50 to 500); R₁ is a monovalent radical conforming to the general formula (CH₂)_{q}L, wherein q is an integer having a value from 1 to 8 and L is selected from the following groups:
—N(R₂)CH₂—CH₂—N(R₂)₂
—N(R₂)₂
—N(R₂)₃A⁻
—N(R₂)CH₂—CH₂—NR₂H₂A⁻
   wherein R₂ is hydrogen, phenyl or aryl, or a saturated hydrocarbon radical, preferably an alkyl radical from about C₁ to about C₂₀, and A is a halide ion. An exemplary silicone polymer is trimethylsilylamodimethicone as shown in the following formula:

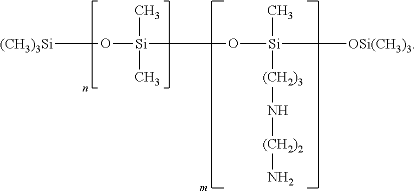

Another exemplary silicone polymer is represented by the general formula:

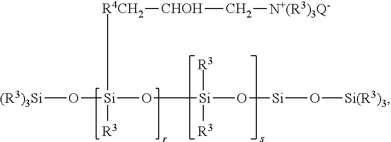

wherein R³ is a monovalent hydrocarbon radical from C₁ to C₁₈, preferably an alkyl or alkenyl, such as methyl; R₄ is a hydrocarbon, preferably a C₁ to C₁₈ alkylene or a C₁₀ to C₁₈ alkyleneoxy, more preferably a C₁ to C₈ alkyleneoxy; Q⁻ is a halide ion, preferably chloride; r is an average statistical value from 2 to 20, preferably from 2 to 8; s is an average statistical value from 20 to 200, preferably from 20 to 50. A suitable polymer of this class is known as UCARE SILICONE ALE 56™, available from Union Carbide. Other suitable silicone materials are disclosed in US Patent Application Publication No. 2007/0039103 A1.

Suitable hydrocarbon oils include straight or branched chain alkanes and alkenes. The chain length may be selected based on desired functional characteristics such as volatility. Suitable hydrocarbon oils may have between 5-20 carbon atoms or, alternately, between 8-16 carbon atoms. Suitable hydrocarbons include pentane, hexane, heptane, decane, dodecane, tetradecane, tridecane, and C₈₋₂₀ isoparaffins as disclosed in U.S. Pat. Nos. 3,439,088 and 3,818,105. Suitable hydrocarbons include isooctane, isododecane, isobexadecane, isoeicosane by Permethyl Corporation under the tradename Permethyl®. Suitable hydrocarbon oils may have greater than about 20 carbon atoms. Examples of such hydrocarbon oils include C₂₄₋₂₈ olefins, C₃₀₋₄₅ olefins, C₂₀₋₄₀ isoparaffins, hydrogenated polyisobutene, polyisobutene, polydecene, hydrogenated polydecene, mineral oil, pentahydrosqualene, squalene, squalane, and mixtures thereof.

Other suitable oils include esters. Suitable esters typically contain at least 10 carbon atoms. These esters include esters with hydrocarbyl chains derived from fatty acids or alcohols (e.g., mono-esters, polyhydric alcohol esters, and di- and tri-carboxylic acid esters). The hydrocarbyl radicals of the esters hereof may include or have covalently bonded thereto other compatible functionalities, such as amides and alkoxy moieties (e.g., ethoxy or ether linkages, etc.). Exemplary esters include, but are not limited to, isopropyl isostearate, hexyl laurate, isohexyl laurate, isohexyl palmitate, isopropyl palmitate, decyl oleate, isodecyl oleate, hexadecyl stearate, decyl stearate, isopropyl isostearate, dihexyldecyl adipate, lauryl lactate, myristyl lactate, cetyl lactate, oleyl stearate, oleyl oleate, oleyl myristate, lauryl acetate, cetyl propionate, and oleyl adipate. Other suitable esters are further described in the Personal Care Product Council's International Cosmetic Ingredient Dictionary and Handbook, Thirteenth Edition, 2010, under the functional category of “Esters.”

Other esters suitable for use in the personal care composition include mono-carboxylic acid esters of the general formula R′COOR, wherein R′ and R are straight or branched chain, saturated or unsaturated alkyl, aryl, and wherein sum of carbon atoms in R′ and R is at least 10, A suitable monoester is alkyl benzoate such as Chans alkyl benzoate.

Other esters suitable for use in the personal care composition include di- and tri-alkyl and alkenyl esters of carboxylic acids, such as esters of C₄ to C₈ dicarboxylic acids (e.g. C₁ to C₂₂ esters, preferably C₁ to C₆, of succinic acid, glutaric acid, and adipic acid). Specific non-limiting examples of di- and tri-alkyl and alkenyl esters of carboxylic acids include isocetyl stearyol stearate, diisopropyl adipate, dibutyl adipate, and tristearyl citrate.

Other esters suitable for use in the personal care composition include those known as polyhydric alcohol esters. Such polyhydric alcohol esters include alkylene glycol esters, such as ethylene glycol mono and di-fatty acid esters, diethylene glycol mono- and di-fatty acid esters, polyethylene glycol mono- and di fatty acid esters, propylene glycol mono- and di-fatty acid esters, polypropylene glycol monooleate, polypropylene glycol 2000 monostearate, ethoxylated propylene glycol monostearate, glyceryl mono- and di-fatty acid esters, polyglycerol poly-fatty acid esters, ethoxylated glyceryl monostearate, 1,3-butylene glycol monostearate, 1,3-butylene glycol distearate, polyoxyethylene polyol fatty acid ester, sorbitan fatty acid esters, and polyoxyethylene sorbitan fatty acid esters.

Still other esters suitable for use in the personal care composition include glycerides, including, but not limited to, mono-, di-, and tri-glycerides. For use in the compositions described herein, the glycerides may be mono, di, and tri-esters of glycerol and long chain carboxylic acids, such as C₁₀ to C₂₂ carboxylic acids. A variety of these types of materials can be obtained from vegetable and animal fats and oils, such as castor oil, safflower oil, cottonseed oil, corn oil, olive oil, cod liver oil, almond oil, avocado oil, palm oil, sesame oil, sweet almond oil, apricot kernel oil, Camelina sativa oil, rapeseed oil, tamanu seed oil, linseed oil, coconut oil, lanolin oil, soybean oil, and the like. Synthetic oils include, but are not limited to, triolein and tristearin glyceryl dilaurate. Other glyceryl esters of fatty acids include fatty acid mono-, di-, and triglycerides which are natural fats or oils that have been modified such as glyceryl stearate, diglyceryl diiosostearate, polyglyceryl-3 isostearate, polyglyceryl-4 isostearate, polyglyceryl-6 ricinoleate, glyceryl dioleate, glyceryl diisotearate, glyceryl tetraisostearate, glyceryl trioctanoate, diglyceryl distearate, glyceryl linoleate, glyceryl myristate, glyceryl isostearate, PEG castor oils, PEG glyceryl oleates, PEG glyceryl stearates, PEG glyceryl tallowates, and the like.

Other suitable oils include fatty amides. Fatty amides include compounds having an amide functional group while being liquid at 25° C. and insoluble in water. In certain embodiments, the fatty amide may have the general formula:

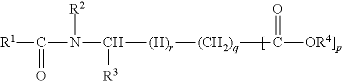

wherein R1 is an optionally functionalized, aliphatic, cycloaliphatic or cyclic, saturated or unsaturated, monovalent hydrocarbon radical containing from 1 to 30 carbon atoms (alternately, from 1 to 22 carbon atoms); R2, R3 and R4, which may be identical or different, are hydrogen or optionally functionalized, aliphatic, cycloaliphatic or cyclic, saturated or unsaturated, monovalent hydrocarbon radicals containing from 1 to 30 carbon atoms, preferably from 1 to 22 carbon atoms; r is 0 or 1; q is an integer from 0 to 2; and p equals 0 or 1. Particular fatty amides include N-acetyl-N-butylaminopropionate, isopropyl N-lauroylsarcosinate, and N,N,-diethyltoluamide. Other suitable fatty amides are disclosed in U.S. Pat. No. 6,872,401.

Other suitable oils include ethers. Suitable ethers include saturated and unsaturated fatty ethers of a polyhydric alcohol, and alkoxylated derivatives thereof. Exemplary ethers include C₄-20 alkyl ethers of polypropylene glycols, and di-C₈-30 alkyl ethers. Suitable examples of these materials include PPG-14 butyl ether, PPG-15 stearyl ether, dioctyl ether, dodecyl octyl ether, and mixtures thereof.

### Emulsifiers

The personal care composition may comprise an emulsifier. An emulsifier is particularly suitable when the composition is in the form of an emulsion or if immiscible materials are being combined. The personal care composition may comprise from about 0.05%, 0.1%, 0.2%, 0.3%, 0.5%, or 1% to about 20%, 10%, 5%, 3%, 2%, or 1% emulsifier. Emulsifiers may be nonionic, anionic or cationic. Non-limiting examples of emulsifiers are disclosed in U.S. Pat. Nos. 3,755,560, 4,421,769, and Mccutcheon's, Emulsifiers and Detergents, 2010 Annual Ed., published by M. C. Publishing Co. Other suitable emulsifiers are further described in the Personal Care Product Council's International Cosmetic Ingredient Dictionary and Handbook, Thirteenth Edition, 2006, under the functional category of “Surfactants—Emulsifying Agents.”

Suitable emulsifying ethers and esters include:

Ethers of polyglycols and of fatty alcohols-including saturated or unsaturated C₁₂₋₃₀) alcohols (e.g., oleyl alcohol, cetyl alcohol, stearyl alcohol or behenyl alcohol) and polyglycols comprising n number of oxyalkylene groups wherein n=an integer from 1 to 200 or, alternately, from 2 to 30 (e.g., 1 to 20 oxyethylene groups). Particular examples include compounds with the INCI names of steareth-n, beheneth-n or oleth-n. Suitable examples include compounds having the INCI names steareth-8, steareth-10, steareth-16, steareth-20, ceteth-10, laureth-4, laureth-3, trideceth-6, ceteareth-5, oleth-10, and beneth-10.

Esters of polyglycols and of fatty acids—including saturated or unsaturated C₁₂-30 fatty acids (e.g., oleic acid, cetylic acid, stearic acid) and polyglycols comprising n number of oxyalkylene groups wherein n=an integer from 1 to 200 or alternately, 1 to 50 (e.g., 1 to 20 oxyethylene groups). Particular examples include compounds with the INCI name PEG-n stearate or PEG-n oleate). Suitable examples include polyethylene glycol-8 monostearate, polyethylene glycol-10, or polyethylene glycol-12 distearate.

Ethers of polyglycols and of fatty alcohols which are glycosylated—including C12-30 alcohols having from 1 to 10 glycosyl groups and polyglycols comprising n number of oxyalkylene groups wherein n=an integer from 1 to 200 (e.g., 1 to 20 oxyethylene groups). A suitable example includes polyoxyethylenated (20 OE) methyl glucose distearate.

Esters of polyglycols and of fatty acids which are glycosylated . . . including C₁₂₋₃₀ fatty acids having from 1 to 10 glycosyl groups and polyglycols comprising a number of oxyalkylene groups wherein n=an integer from 1 to 200 (e.g., 1 to 20 oxyethylene groups).

Ethers of C₁₂-30 alcohols and of glycerol or of polyglycero—A suitable example includes polyglyceryl-3 cetyl ether, such as Chimexane NL from Chimex, Esters of C12-30 fatty acids and of glycerol or of polyglycerol—including esters comprising from 1 to 10 glycerol groups. Particular examples include hexa-glyceryl monosterate, diglyceryl distearate, tetraglyceryl tristearate, decaglyceryl decastearate, diglyceryl monostearate, hexaglyceryl tristearate, decaglyceryl pentastearate, the ester of glycerol and of palmitic and stearic acids, and glyceryl mono- and dibehenate.

Ethers of oxyalkylene-modified C₁₂₋₃₀ alcohols and of glycerol or polyglycerol.

Ethers of C₁₂-30 fatty alcohols comprising and of sucrose or glucose-Suitable examples include compounds with the INCI names of C₁₂₋₁₈ alkylglucoside, C₁₂₋₂₀ alkylglucoside (e.g., Montanov L from Seppic), cetearyl glucoside (e.g., a mixture with cetearyl alcohol under the reference Montanov 68 from Seppic), myristyl glucoside (e.g., a mixture with myristyl alcohol under the reference Montanov 14 from Seppic) or cetearyl glucoside (e.g., Tegocare CG 90 from Evonik Goldschmidt),

Esters of sucrose and of C₁₂₋₃₀ fatty acids-Particular examples include sucrose distearate or sucrose tristearate, sucrose cocoate, sucrose dilaurate, sucrose distearate, sucrose hexaerucate, sucrose hexapalmitate, sucrose laurate, sucrose mortierellate, sucrose myristate, sucrose oleate, sucrose palmitate, sucrose pentaerucate, sucrose polybehenate, sucrose polycottonseedate, sucrose polylaurate, sucrose polylinoleate, sucrose polyoleate, sucrose polypalmate, sucrose polysoyate, sucrose polystearate, sucrose ricinoleate, sucrose stearate, sucrose tetraisostearate, and sucrose trilaurate. A suitable example includes the mixture of esters (mono- and polyesters) of stearic acid and of sucrose sold as Crodesta FI 10 by Croda.

Esters of pentaerythritol and of C₁₂-30 fatty acids—Particular examples include pentaerythritol tetrastearate.

Esters of sorbitol and/or of sorbitan and of C₁₂₋₃₀ fatty acids—Particular examples include sorbitan monostearate, sorbitan tristearate, or sorbitan laurate, such as Span 20 from Uniqema,

Ethers of sorbitol and/or of sorbitan and of alkoxylated sorbitan—Suitable examples include sorbeth-8 beeswax or sorbeth-20 beeswax from Nikko Chemical.

Ethers of polyglycols and of cholesterol—Particular examples include choleth-3, choleth-10 (such as Emalex CS-10 from Nihon Emulsion Company), choleth-15 (such as Emalex CS-15 from Nihon Emulsion Company) or choleth-20 (such as Emalex CS-20 from Nihon Emulsion Company).

Esters of C₁₂₋₃₆ fatty acids and of alkoxylated ethers of sorbitol and/or of Suitable examples include polysorbate-60, polysorbate-61, sorbeth-3 isostearate, polyoxyethylenated 4 OE sorbitan monostearate, and polyoxyethylenated 20 OE sorbitan tristearate.

Linear or branched type silicone emulsifiers may also be used. Particularly useful polyether modified silicones include KF-6011, KF-6012, KF-6013, KF-6015, KF-6015, KF-6017, KF-6043, KF-6028, and KF-6038 from Shin Etsu. Also particularly useful are the polyglycerolated linear or branched siloxane emulsifiers including KF-6100, KF-6104, and KF-6105 from Shin Etsu. Exemplary materials include materials with the following International Nomenclature of Cosmetic Ingredients (INCD) designations: Bis-Butyldimethicone Polyglyceryl-3; Bis-PEG/PPG-14/14 Dimethicone; Bis-butyldimethicone Polyglyceryl-3; Bis-isobutyl PEG/PPG-10/7 Dimethicone copolymer; Bis-PEG/PPG-18/6 Dimethicone; Bis-PEG/PPG-20/20 Dimethicone; Bis-PEG/PPG-16/16 PEG/PPG-16/16 Dimethicone; Bist(PPG-7 Undeceneth-21-Dimethicone; Cetyl Dimethicone PEG-7 Acetate; Cetyl PEG-8 Dimethicone; Cetyl PEG/PPG-15/16 Butyl Ether Dimethicone; Cetyl PEG/PPG-15/15 Butyl Ether Dimethicone; Cetyl PEG/PPG-7/3 Dimethicone; Cetyl PEG/PPG-10/1 Dimethicone; Dimethicone PEG-15 Acetate; Dimethicone PEG-7 Cocoate; Dimethicone PEG-7 Phosphate; Dimethicone PEG-IO Phosphate; Dimethicone PEG/PPG-7/4 Phosphate; Dimethicone PEG/PPG-12/4 Phosphate; Dimethicone PEG-7 Undecylenate; Lauryl Dimethicone PEG-IO Phosphate; Isopolyglyceryl-3 Dimethicone; Isopolyglyceryl-3 Dimethiconol; Isostearyl Carboxyldecyl PEG-8 Dimethicone; Lauryl Methicone PEG-10 Phosphate; Lauryl PEG-8 Dimethicone; Lauryl PEG-10 Methyl Ether Dimethicone; Lauryl PEG/PPG-18/18 Methicone; PEG-6 Methyl Ether Dimethicone; PEG-7 Methyl Ether Dimethicone; PEG-9 Methyl Ether Dimethicone; PEG-10 Methyl Ether Dimethicone; PEG-11 Methyl Ether Dimethicone; PEG-11 Methyl Ether Dimethicone; PEG-32 Methyl Ether Dimethicone; PEG-PEG/PPG-28/21 Acetate Dimethicone; PEG/PPG-22/22 Butyl Ether Dimethicone; PEG/PPG-23/23 Butyl Ether Dimethicone; PEG/PPG-24/18 Butyl Ether Dimethicone; PEG/PPG-3/10 Dimethicone; PEG/PPG-4/12 Dimethicone; PEG/PPG-6/11 Dimethicone; PEG/PPG-8/14 Dimethicone; PEG/PPG-12/16 Dimethicone; PEG/PPG-12/18 Dimethicone; PEG/PPG-14/4 Dimethicone; PEG/PPG-15/5 Dimethicone; PEG/PPG-15/15 Dimethicone; PEG/PPG-16/2 Dimethicone; PEG/PPG-16/8 Dimethicone; PEG/PPG-17/18 Dimethicone; PEG/PPG-18/12 Dimethicone; PEG/PPG-19/19 Dimethicone; PEG/PPG-20/6 Dimethicone; PEG/PPG-20/15 Dimethicone; PEG/PPG-20/20 Dimethicone; PEG/PPG-20/29 Dimethicone; PEG/PPG-22/23 Dimethicone; PEG/PPG-22/24 Dimethicone; PEG/PPG-25/25 Dimethicone; PEG/PPG-27/27 Dimethicone; PEG/PPG-30/10 Dimethicone; PEG/PPG-10/3 Oleyl Ether Dimethicone; PEG-8 trisiloxane; Polyglyceryl-3 Polydimethylsiloxyethyl Dimethicone; PPG-12 Butyl Ether Dimethicone; Silicone Quaternium-17; TEA-Dimethicone PEG-7 Phosphate; and mixtures thereof.

Emulsifiers also include emulsifying silicone elastomers. Suitable emulsifying silicone elastomers may include at least one polyalkyl ether or polyglycerolated unit. These cross-linked elastomers may also be co-modified to include alkyl substituents. Suitable formation techniques are described in U.S. Pat. Nos. 5,236,986; 5,412,004; 5,837,793; and 5,811,487. Polyoxyalylenated emulsifying silicone elastomers that may be used in at least one embodiment of the disclosure include those sold by Shin-Etsu Silicones under the names KSG-21, KSG-20, KSG-30, KSG-31, KSG-32, KSG-33; KSG-210 (dimethicone/PEG-10/15 crosspolymer dispersed in dimethicone); KSG-310 (PEG-15 lauryl dimethicone crosspolymer); KSG-320 (PEG-15 lauryl dimethicone crosspolymer dispersed in isododecane); KSG-330 (PEG-15 lauryl dimethicone crosspolymer dispersed in triethylhexanoin), KSG-340 (PEG-10 lauryl dimethicone crosspolymer and PEG-15 lauryl dimethicone crosspolymer). Other silicone emulsifying elastomers are supplied by Dow Corning™, including PEG-12 dimethicone crosspolymers (DC 9010 and 9011). Other suitable silicone emulsifiers sold by Dow Corning include DC9010 and DC9011.

Polyglycerolated emulsifying silicone elastomers are disclosed in PCT/WO 2004/024798. Such elastomers include Shin-Etsu's KSG series, such as KSG-710 (dimethicone/polyglycerin-3 crosspolymer dispersed in dimethicone); or lauryl dimethicone/polyglycerin-3 crosspolymer dispersed in a variety of solvent such as isododecane, dimethicone, triethylhexanoin, available as KSG-810, KSG-820, KSG-830, or KSG-840 from Shin-Etsu.

Another suitable crosslinked silicone elastomer emulsifier is dimethicone/PEG-10/15 crosspolymer, which provides excellent aesthetics due to its elastomeric backbone, but also excellent emulsification properties. Further examples of crosslinked organosiloxane emulsifiers include, but are not limited to dimethicone/dimethicone PEG/PPG 15 crosspolymer; dimethicone PEG-10 crosspolymer; dimethicone PEG-10/15 crosspolymer; dimethicone PEG-15 crosspolymer; dimethicone polyglycerin-3 crosspolymer; dimethicone PPG-20 crosspolymer; lauryl dimethicone PEG-15 crosspolymer; lauryl dimethicone polyglycerin-3 crosspolymer; PEG-8 dimethicone polysorbate-20 crosspolymer; PEG-10 dimethicone/vinyl dimethicone crosspolymer; PEG-10 lauryl dimethicone crosspolymer; PEG-15/lauryl dimethicone crosspolymer; PEG-15 laurylpolydimethylsiloxy ethyl crosspolymer; and mixtures thereof.

It should be recognized that silicone elastomers may be supplied pre-swollen with a solvent. With a pre-swollen swollen elastomer, the weight percentages recited for emulsifier use (i.e., from about 0.05% to about 20%, from about 0.1% to about 10%, from about 0.5% to about 5%, or from about 1% to about 3% emulsifier) are of the elastomer alone (i.e., excluding the weight of the solvent).

### Structuring Agent

The personal care composition may comprise a structuring agent. Structuring agents may be used to increase viscosity, thicken, solidify, or provide solid or crystalline structure to the personal care composition. The structuring agent may be used to suspend or disperse the abrasive particles. Structuring agents are typically grouped based on solubility, dispersibility, or phase compatibility. Examples of aqueous or water structuring agents include polymeric agents, natural or synthetic gums, polysaccharides, and the like. In one embodiment, the composition may comprises from about 0.0001%, 0.001%, 0.01%, 0.05%, 0.1%, 0.5%, 1%, 2%, 3%, 5% to about 25%, 20%, 10%, 7%, 5%, 4%, or 2%, by weight of the composition, of one or more structuring agents.

Polysaccharides and gums may be used as aqueous phase thickening agents. Examples of such polysaccharides and gums include naturally derived materials such as agar, agarose, alicaligenes polysaccharides, algin, alginic acid, acacia gum, amylopectin, chitin, dextran, cassia gum, cellulose gum, gelatin, gellan gum, hyaluronic acid, hydroxyethyl cellulose, methyl cellulose, ethyl cellulose, pectin, sclerotium gum, xanthan gum, pectin, trehelose, gelatin, ammonium alginate, calcium alginate, calcium carrageenan, carnitine, carrageenan, guar gum, guar hydroxypropyltrimonium chloride, hyaluronic acid, hydroxypropyl chitosan, hydroxypropyl guar, karaya gum, kelp, locust bean gum, natto gum, potassium alginate, potassium carrageenan, propylene glycol alginate, sodium carboyxmethyl dextran, sodium carrageenan, tragacanth gum, and mixtures thereof. Suitable polysaccharides include alkyl hydroxyalkyl cellulose ethers such as cetyl hydroxyethylcellulose, which is the ether of cetyl alcohol and hydroxyethylcellulose. This material is sold under the tradename Natrosol® Plus CS from Ashland Aqualon Functional Ingredients. Other useful polysaccharides include scleroglucans comprising a linear chain of (1-3) linked glucose units with a (1-6) linked glucose every three units, a commercially available example of which is Clearogel™ CS 11 from M.M.P., Inc.

Suitable classes of polymeric structuring agents include but are not limited to carboxylic acid polymers, polyacrylamide polymers, sulfonated polymers, high molecular weight polyalkylglycols or polyglycerins, copolymers thereof, hydrophobically modified derivatives thereof, and mixtures thereof.

Carboxylic acid polymers include carbomers. These polymers are crosslinked compounds containing one or more monomers derived from acrylic acid, substituted acrylic acids, and salts and esters of these acrylic acids and the substituted acrylic acids, wherein the crosslinking agent contains two or more carbon-carbon double bonds and is derived from a polyhydric alcohol. Suitable materials includes include the Carbopol® 900 series (e.g., Carbopol® 945. Carbopol® 940, Carbopol® 950, Carbopol® 954, Carbopol® 980. Carbopol® 951 and Carbopol® 981 from Noveon, Inc) and the Carbopol® Ultrez series (e.g., Carbopol®) Ultrez 10 polymer, Carbopol® Ultrez 20 polymer, and Carbopol® Ultrez 21 polymer). Other suitable carboxylic acid polymeric agents include copolymers of Co10-30 alkyl acrylates with one or more monomers of acrylic acid, methacrylic acid, or one of their short chain (i.e., C₁₋₄ alcohol) esters, wherein the crosslinking agent is an allyl ether of sucrose or pentaerytritol. These copolymers are known as acrylates/C₁₀-30 alkyl acrylate crosspolymers and are commercially available as Carbopol® 1342. Carbopol® 1382, PEMULEN™ TR-1, and PEMULEN™ TR-2, from Noveon, Inc.

Sulfonated polymers include polymers and copolymers containing 2-acrylamido-2-methylpropane sulfonic acid (i.e., AMPS or acryloyldimethyl tauric acid) and salts thereof. Exemplary AMPS structurants include sodium acrylate/sodium acryloyldimethyl taurate copolymer available as SIMULGEL® EG and SIMULGEL® EPG or hydroxyethyl acrylate/sodium acryloyldimethyl taurate copolymer available as SIMULGEL® NS, SIMULGEL® FL, and SIMULGEL® I-NS 100; which are available from Seppic Corporation (Fairfield, N.J.). Another suitable sulfonated polymer is sodium polyacryloyldimethyl taurate available as Simulgel® 800 from Seppic Corporation (Fairfield, N.J.). Other suitable sulfonated polymers include acrylamide/sodium acryloyldimethyltaurate/acrylic acid copolymer available as Acudyne™ SCP from Rohm and Haas Company, Inc.; acrylamide/sodium acryloyldimethyltaurate copolymer available as Simulgel® 600 from Seppic; ammonium acryloyldimethyltaurate/beheneth-25 methacrylate crosspolymer available as Aristoflex® BLV from Clariant International Ltd.; ammonium acryloyl dimethyltaurate/carboxyethyl acrylate crosspolymer available as Aristoflex® TAC from Clariant International Ltd.; ammonium acryloyldimethyltaurate/vinylpyrrolidone copolymer available as Aristoflex® AVC from Clariant International Ltd.; dimethylacrylamide/sodium acryloyldimethyltaurate crosspolymer available as SUpolymer G-1 from Toho Chemical Industry Co., Ltd.; sodium acrylate/acryloyldimethyltaurate/dimethylacrylamide crosspolymer available as Sepinov™ PBS from Seppic; and sodium acryloyldimethyltaurate/VP Crosspolymer available as Aristoflex® AVS from Clariant International, Ltd. Additional sulfonated structurants are described in US Patent Application Publication Nos. 2007/0140993 (identified as gelling agent in the form of a copolymer of acryloyl dimethyl tauric acid or a salt thereof) and 2006/0147396 A1 (identified as “polymer containing at least one sulpho-functional monomer”).

Acrylamide polymers and copolymers include SEPIGEL® 305 from Seppic Corporation (Fairfield, N.J.), which is designated by the Personal Care Product Council's International Cosmetic Ingredient Dictionary and Handbook, Thirteenth Edition, 2010, as “polyacrylamide and isoparaffin and laureth-7.” Other polyacrylamide polymers include multi-block copolymers of acrylamides and substituted acrylamides with acrylic acids and substituted acrylic acids. Commercially available examples of these multi-block copolymers include HYPAN® SR150H, SS500V, SS500 W, SSSA100H, from Lipo Chemicals, Inc., (Patterson, N.J.).

High molecular weight polyalkylglycols or polyglycerins may be used as structuring agents. Suitable materials include polyethylene glycols (PEG) derivatives and polypropylene glycols (PPO) derivatives with an n degree of polymerization. n may be from 50 to 200,000. Other suitable materials are polyglycerins having repeating glycerin moieties where the number of repeating moieties ranges from about 15 to about 200, or from about 20 to about 100. Examples of suitable polyglycerins include those having the INCI names polyglycerin-20, polyglycerin-40, and the like.

Examples of oil structuring agents include silicone and organic based materials. Suitable ranges of oil structuring agents are from about 0.01%, 0.05%, 0.1% 0.5%, 1%, 2.5%, 5%, or 10% to about 30%, 25%, 20%, 15%, 10%, or 5%. Suitable oil phase structuring agents may be silicone based, such as silicone elastomers, silicone gums, silicone waxes, linear silicones having a degree of polymerization allowing the silicone to increase the viscosity of the oil phase. Examples of silicone structuring agents include, but are not limited to, silicone elastomers, silicone gums, and silicone waxes.

Silicone elastomers suitable for use in the compositions of the disclosure include those that are formed by addition reaction-curing, by reacting an SiH-containing diorganosiloxane and a organopolysiloxane having terminal olefinic unsaturation, or an alpha-omega diene hydrocarbon, in the presence of a platinum metal catalyst. Such elastomers may also be formed by other reaction methods such as condensation-curing organopolysiloxane compositions in the presence of an organotin compound via a dehydrogenation reaction between hydroxyl-terminated diorganopolysiloxane and SiH-containing diorganopolysiloxane or alpha omega diene; or by condensation-curing organopolysiloxane compositions in the presence of an organotin compound or a titanate ester using a condensation reaction between an hydroxyl-terminated diorganopolysiloxane and a hydrolysable organosiloxane; peroxide-curing organopolysiloxane compositions which thermally care in the presence of an organoperoxide catalyst. Cross-linked organopolysiloxane elastomers useful in the present disclosure and processes for making them are further described in U.S. Pat. Nos. 4,970,252, 5,760,116, and 5,654,362, 6,524.598, and 6,696,049. It is particularly desirable to incorporate silicone elastomers into the compositions of the disclosure because they provide excellent “feel” to the composition, are very stable in cosmetic formulations, and relatively inexpensive.

Suitable silicone elastomers may be in the powder form, or dispersed or solubilized in solvents such as volatile or nonvolatile silicones, or silicone compatible vehicles such as hydrocarbons or esters. Examples of silicone elastomer powders include vinyl dimethicone/methicone silesquioxane crosspolymers like KSP-100, KSP-101, KSP-102, KSP-103, KSP-104, KSP-105, available from Shin-Etsu, hybrid silicone powders that contain a fluoroalkyl group like KSP-200, available from Shin-Etsu, which is a fluoro-silicone elastomer, and hybrid silicone powders that contain a phenyl group such as KSP-300, available from Shin-Etsu, which is a phenyl substituted silicone elastomer; and DC 9506 available from Dow Corning.

Examples of silicone elastomer dispersed in a silicone compatible vehicle include dimethicone/vinyl dimethicone crosspolymers supplied by a variety of suppliers including Dow Corning Corporation under the tradenames DC9040 or DC9041. Momentive under the tradename SFE 839, or Shin-Etsu Silicones under the tradenames KSG-15, 16, 18. KSG-15 has the INCI name cyclopentasiloxane (and) dimethicone/vinyl dimethicone crosspolymer. KSG-18 has the INCI name diphenylsiloxy phenyl trimethicone (and) dimethicone/phenyl vinyl dimethicone crosspolymer. Silicone elastomers may also be purchased from Grant Industries under the Gransil trademark. Other suitable silicone elastomers have long chain alkyl substitutions such as lauryl dimethicone/vinyl dimethicone crosspolymers supplied by Shin Etsu under the tradenames KSG-31, KSG-32, KSG-41, KSG-42, KSG-43, and KSG-44.

Silicone gums are another oil phase structuring agent. The silicone gum typically has a viscosity ranging from about 500,000 to 100 million est at 25° C., from about 600,000 to 20 million, from about 600,000 to 12 million est. The silicone gums that are used in the compositions include, but are not limited to, those of the general formula wherein:

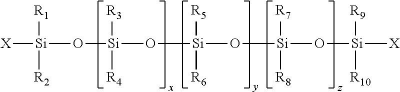

R₁ to R₁₀ are each independently hydrogen, an alkyl having 1 to 30 carbon atoms, aryl, or aralkyl; and X is H, OH, or a C1-30 alkyl or vinyl. x, y, or z may be zero with the proviso that (x+y+z)≥1.

Such silicone gums may be purchased in pure form from a variety of silicone manufacturers including Wacker-Chemie or Dow Corning, and the like. Silicone gums include those sold by Wacker-Belsil under the trade names CM3092, Wacker-Belsil 1000, or Wacker-Belsil DM 3096. A silicone gum where X is OH, also referred to as dimethiconol, is available from Dow Corning Corporation under the trade name 1-1254 Fluid, 2-9023 Fluid, and 2-9026 Fluid. The silicone gum may also be purchased in the form of a solution or dispersion in a silicone compatible vehicle such as volatile or nonvolatile silicone. An example of such a mixture may be purchased from Bamet Silicones under the HL-88 tradename, having the INCI name dimethicone. Another example is a mixture of dimethiconol and volatile or nonvolatile silicone available from the Dow Corning Corporation as tradename 1401 Fluid, 1403 Fluid, and 1501 Fluid.

Another type of oily phase structuring agent includes silicone waxes. Silicone waxes may be referred to as alkyl silicone waxes which and are semi-solids or solids at room temperature. The term “alkyl silicone wax” means a polydimethylsiloxane having a substituted long chain alkyl (such as C16 to 30) that confers a semi-solid or solid property to the siloxane. Examples of such silicone waxes include stearyl dimethicone, which may be purchased from Evonik Goldschmidt GmbH under the tradename Abil Wax 9800 or from Dow Corning under the tradename 2503. Another example is bis-stearyl dimethicone (which may be purchased from Gransil Industries under the tradename Gransil A-18), behenyl dimethicone, or behenoxy dimethicone.

Other suitable structuring agents include polyamides and polysilicone-polyamide copolymers. Suitable polysilicone-polyamide copolymers are disclosed in U.S. Patent Application Publication No. 2004/0170586. A specific example of such copolymers is nylon 611/dimethicone copolymers by Dow Corning under the tradename Dow Corning 2-8178. Also suitable are polyamides such as those purchased from Arizona Chemical under the Uniclear™ and Sylvaclear® including Sylvaclear® A200V or A2614V (INCI name: ethylenediamine/hydrogenated dimer dilinoleate copolymer/bis-di-C₁₄₋₁₈ alkyl amide); Sylvaclear® AF1900V and Sylvaclear® PA1200V (INCI name: Polyamide-3); Sylvaclear® C₇₅V (INCI name: bis-stearyl ethylenediamine/neopentyl glycol/stearyl hydrogenated dimer dilinoleate copolymer); Sylvaclear® PE400V (INCI name: Polyamide-6); Sylvaclear®) WF 1500V (INCI name: Polyamide-4), or Uniclear™ 100 VG (INCI name: ethylenediamine/stearyl dimer dilinoleate copolymer; or ethylenediamine/stearyl dimer ditallate copolymer).

Other oil phase structuring agents may include one or more natural or synthetic waxes such as animal, vegetable, or mineral waxes. Generally such waxes have a melting point ranging from about 25° C. to 125° C., and alternatively from about 30° C. to about 100° C. Non-limiting examples of suitable waxes include silicone waxes, fatty esters, for example cetyl and/or stearyl esters, acacia, beeswax, ceresin, flower wax, citrus wax, carnauba wax, jojoba wax, japan wax, polyethylene, microcrystalline, rice bran, lanolin wax, mink, montan, bayberry, ouricury, ozokerite, palm kernel wax, paraffin, avocado wax, apple wax, shellac wax, clary wax, spent grain wax, candelilla, grape wax, polyalkylene glycol derivatives thereof (for example PEG6-20 beeswax, or PEG-12 carnauba wax) and mixtures of any of the aforementioned waxes. In one embodiment, the wax is a polyethylene wax, and alternatively is a polyethylene wax having a melting point of less than 120° C., alternatively less than 95 C, and alternatively less than 85° C.

Non-limiting examples of suitable silicone waxes are disclosed in U.S. Pat. Nos. 5,413,781 and 5,725,845, and further include alkylmethyl polysiloxanes, C₁₀-C₆₀ alkyl dimethicones, and mixtures thereof. Alternatively, the silicone wax may be a C₁₆-C₂₈ alkyl dimethicone wax. Other suitable silicone waxes include, but are not limited to stearoxydimethicone, behenoxy dimethicone, stearyl dimethicone, cetearyl dimethicone, cetyl dimethicone, and mixtures thereof.

Other structuring agents are natural or synthetic montmorillonite minerals such as hectorite, bentonite, and quaternized derivatives thereof, which are obtained by reacting the minerals with a quaternary ammonium compound (e.g., stearalkonium bentonite and stearalkonium hectorite).

Other structuring agents are silicas, silicates, silica silylate, and alkali metal or alkaline earth metal derivatives thereof. These silicas and silicates are generally found in the particulate form and include silica, silica silylate, magnesium aluminum silicate, and the like.

### Additional Ingredients

The personal care compositions may comprise one or more additional components to provide an efficacious and/or consumer desirable product. For example, the composition can include other actives or agents. For instance, suitable optional actives and agents may include an active or agent selected from a group consisting of sugar amines, vitamins, oil control agents, photosterols, hexamidine compounds, tightening agents, anti-wrinkle actives, anti-atrophy actives, flavonoids, N-acyl amino acid compounds, retinoids, peptides, particulate materials, UV actives, photostabilizers, anti-cellulite agents, desquamation actives, anti-acne actives, anti-oxidants, radical scavengers, conditioning agents, anti-inflammatory agents, tanning actives, skin lightening agents, botanical extracts, antimicrobial actives, antifungal actives, antibacterial actives, antiperspirant actives, sensates, preservatives, anti-dandruff actives, substantivity polymers, detersive surfactants, and combinations thereof. Suitable non-limiting examples are discussed in more detail below.

### 1. Sugar Amines

The personal care compositions of the present disclosure can comprise a sugar amine, which is also known as amino sugar. Sugar amine compounds useful in the present disclosure can include those described in PCT Publication WO 02/076423 and U.S. Pat. No. 6,159,485. In one embodiment, the composition may comprise from about 0.001%, 0.01%, 0.05%, 0.1%, 0.5%, or 1% to about 10%, 7.5%, or 2% by weight of the composition, of one or more sugar amine.

Sugar amines can be synthetic or natural in origin and can be used as pure compounds or mixtures of compounds (e.g., extracts from natural sources or mixtures of synthetic materials). For example, glucosamine is generally found in many shellfish and can also be derived from fungal sources. As used herein, “sugar amine” includes isomers and tautomers of such and its salts (e.g., HCl salt) and is commercially available from Sigma Chemical Co.

Examples of sugar amines that are useful herein include glucosamine, N-acetyl glucosamine, mannosamine, N-acetyl mannosamine, galactosamine, N-acetyl galactosamine, their isomers (e.g., stereoisomers), and their salts (e.g., HCl salt). Preferred for use herein are glucosamine, particularly D-glucosamine and N-acetyl glucosamine, particularly N-acetyl-D-glucosamine.

### 2. Vitamins

In some embodiments, the composition may comprise from about 0.001%, 0.01%, 0.05%, 0.1%, 0.5%, or 1% to about 10%, 7, 5%, or 2%, by weight of the composition, of one or more vitamins. “Vitamins” means vitamins, pro-vitamins, and their salts, isomers and derivatives. Non-limiting examples of suitable vitamins include: vitamin B compounds (including B1 compounds, B2 compounds, B3 compound, B5 compounds, such as panthenol or “pro-B5”, pantothenic acid, pantothenyl; B6 compounds, such as pyroxidine, pyridoxal, pyridoxamine; carnitine, thiamine, riboflavin); vitamin D compounds; vitamin K compounds; vitamin E compounds, or tocopherol, including tocopherol sorbate, tocopherol acetate, other esters of tocopherol and tocopheryl compounds; vitamin C compounds, including ascorbate, ascorbyl esters of fatty acids, and ascorbic acid derivatives, for example, ascorbyl phosphates such as magnesium ascorbyl phosphate and sodium ascorbyl phosphate, ascorbyl glucoside, and ascorbyl sorbate; and vitamin F compounds, such as saturated and/or unsaturated fatty acids.

In some embodiments, the personal care compositions do not comprise any vitamin A compounds, or all natural and/or synthetic analogs of Vitamin A, including retinoids, retinol, retinyl acetate, retinyl palmitate, retinoic acid, retinaldehyde, retinyl propionate, carotenoids (pro-vitamin A), or other compounds which possess the biological activity of Vitamin A.

In certain embodiments, the personal care compositions comprise a vitamin B3 compound.

Vitamin B3 compounds are particularly useful for regulating skin conditions, as described in U.S. Pat. No. 5,939,082, the disclosure of which is incorporated herein in its entirety by reference. In one embodiment, the composition may comprise from about 0.001%, 0.01%, 0.05%, 0.1%, 0.5%, or 1% to about 50%, 20%, 10%, 7%, or 5%, by weight of the composition, of the vitamin B3 compound.

As used herein, “vitamin B3 compound” means a compound having the formula:

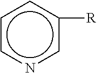

wherein R is —CONH₂ (i.e., niacinamide), —COOH (i.e., nicotinic acid) or —CH₂OH (i.e., nicotinyl alcohol); derivatives thereof; and salts of any of the foregoing.

Exemplary derivatives of the foregoing vitamin B3 compounds include nicotinic acid esters, including non-vasodilating esters of nicotinic acid (e.g., tocopherol nicotinate, myristyl nicotinate), nicotinyl amino acids, nicotinyl alcohol esters of carboxylic acids, nicotinic acid N-oxide and niacinamide N-oxide.

In certain embodiments, the personal care compositions comprise a vitamin C compound. In some embodiments, the vitamin C compound is a vitamin C derivative.

In some embodiments, the vitamin C derivative is ascorbyl palmitate having the formula:

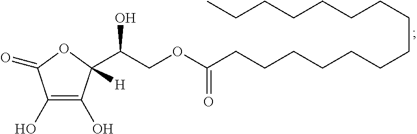 [SMILES: CCCCCCCCCCCCCCCC(=O)OC[C@H](O)[C@H]1OC(=O)C(O)=C1O]

derivatives thereof; and salts of any of the foregoing.

In some embodiments, the vitamin C derivative is magnesium ascorbyl phosphate having the formula:

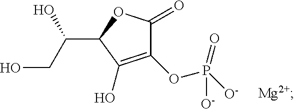 [SMILES: O=C1O[C@H]([C@@H](O)CO)C(O)=C1OP(=O)([O-])[O-]]

derivatives thereof; and salts of any of the foregoing.

In some embodiments, the vitamin C derivative is ethyl ascorbic acid having the formula:

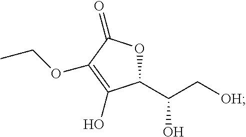

derivatives thereof; and salts of any of the foregoing.

### 3. Phytosterols

The personal care compositions may comprise a phytosterol. For example, one or more phytosterols can be selected from the group consisting of—sitosterol, campesterol, brassicasterol, A5-avennasterol, lupenol, α-spinasterol, stigmasterol, their derivatives, analogs, and combinations thereof. In certain embodiments, the phytosterol is selected from the group consisting of 3-sitosterol, campesterol, brassicasterol, stigmasterol, their derivatives, and combinations thereof. In a select embodiment, the phytosterol is stigmasterol.

Phytosterols can be synthetic or natural in origin and can be used as essentially pure compounds or mixtures of compounds (e.g., extracts from natural sources). Phytosterols are generally found in the unsaponifiable portion of vegetable oils and fats and are available as free sterols, acetylated derivatives, sterol esters, ethoxylated or glycosidic derivatives. More preferably, the phytosterols are free sterols. As used herein, “phytosterol” includes isomers and tautomers of such and is commercially available from Aldrich Chemical Company, Sigma Chemical Company, and Cognis.

In one embodiment, the composition may comprise from about 0.0001%, 0.001%, 0.01%, 0.05%, 0.1%, 0.5%, or 1% to about 25%, 20%, 10%, 7%, 5%, or 3%, by weight of the composition, of one or more phytosterol.

### 4. Hexamidine Compounds

The personal care compositions may include hexamidine compounds, its salts, and derivatives. As used herein, “hexamidine compound” means a compound having the formula:

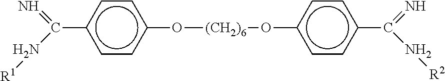

wherein R¹ and R² are optional or are organic acids (e.g., sulfonic acids, etc.).

In one embodiment, the composition may comprise from about 0.0001%, 0.001%, 0.01%, 0.05%, 0.1%, 0.5%, or 1% to about 25%, 20%, 10%, 7%, 5%, or 3%, by weight of the composition, of one or more hexamine compounds.

As used herein, hexamidine derivatives include any isomers and tautomers of hexamidine compounds including but not limited to organic acids and mineral acids, for example sulfonic acid, carboxylic acid, etc. In a select embodiment, the hexamidine compounds include hexamidine diisethionate, commercially available as Eleastab® HP100 from Laboratories Serobiologiques.

### 5. Tightening Agents

The personal care composition may comprise a tightening agent. A tightening agent is a compound capable of having a tightening effect on keratinous tissues and, typically, on skin. Suitable tightening agents may be chosen from plant or animal proteins and their hydrolysates such as maize, rye, wheat, buckwheat, sesame, spelt, pea, bean, lentil, soybean and lupin; polysaccharides of natural origin including (i) polyholosides, for example, in the form of starch derived especially from rice, maize, potato, cassava, peas, wheat, oats, etc. or in the form of carrageenans, alginates, agars, gellans, cellulose polymers and pectins, advantageously as an aqueous dispersion of gel microparticles, and (ii) latices composed of shellac resin, gum sandarac, dammars, elemis, copals, cellulose compounds, and mixtures thereof; mixed silicates including phyllosilicates and in particular laponites; colloidal particles of inorganic fillers such as silica/alumina colloidal particles such as those sold under then tradename LUDOX® by W.R. Grace & Co.; synthetic polymers such as polyurethane latices or acrylic/silicone latices, in particular those described in US Patent Application Publication No. 2002/0131948, including propylthio(polymethyl acrylate), propylthio(polymethyl methacrylate) and propylthio(polymethacrylic acid) grafted polydimethylsiloxane, propyl-thio(polyisobutyl methacrylate) and propylthio(poly-methacrylic acid) grafted polydimethylsiloxane (available under the tradenames VS 80, VS 70 and 1.021 from 3M); and mixtures thereof.

The personal care composition may comprise from about 0.0001%, 0.001%, 0.01%, 0.05%, 0.19%, 0.5%, or 1% to about 30%, 25%, 20%, 10%, 79%, 5%, or 3% by weight of the composition, of one or more tightening agent.

### 6. Anti-Wrinkle Actives/Anti-Atrophy Actives

The personal care compositions of the present disclosure can comprise a one or more anti-wrinkle actives or anti-atrophy actives. Exemplary anti-wrinkle/anti-atrophy actives suitable for use in the compositions of the present disclosure include dialkanoyl hydroxyproline compounds, hydroxy acids (e.g., glycolic acid, lactic acid, lactobionic acid), keto acids (e.g., pyruvic acid), phytic acid, lysophosphatidic acid, stilbenes, cinnamates, resveratrol, kinetin, zeatin, dimethylaminoethanol, peptides from natural sources (e.g., soy peptides), and salts of sugar acids (e.g., Mn gluconate, Zn gluconate). In one embodiment, the composition may comprise from about 0.0001%, 0.001%, 0.01%, 0.05%, 0.1%, 0.5%, or 1% to about 30%, 25%, 20%, 10%, 7%, 5%, or 3% by weight of the composition, of one or more anti-wrinkle/anti-atrophy compounds.

Suitable dialkanoyl hydroxyproline compounds of the present disclosure can include those corresponding to the following chemical formula:

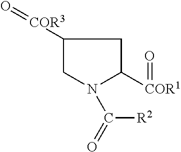

wherein R¹ is H, X, C₁-C₂₀ straight or branched alkyl,
X is metals (Na, K, Li, Mg, Ca) or amines (DEA, TEA);
R₂ is C₁-C₂₀ straight or branched alkyl;
R₃ is C₁-C₂₀ straight or branched alkyl.

Suitable derivatives include but are not limited to esters, for example fatty esters, including, but not limited to tripalmitoyl hydroxyproline and dipalmityl acetyl hydroxyproline. A particularly useful compound is dipalmitoyl hydroxyproline. As used herein, dipalmitoyl hydroxyproline includes any isomers and tautomers of such and is commercially available under the tradename Sepilift DPHP® from Seppic, Inc. Further discussion of dipalmitoyl hydroxyproline appears in PCT Publication WO 93/23028. Preferably, the dipalmitoyl hydroxyproline is the triethanolamine salt of dipalmitoyl hydroxyproline as discussed in U.S. Pat. No. 7,285,570.

### 7. Flavonoids

The personal care compositions of the present disclosure can comprise a flavonoid compound. Flavonoids are broadly disclosed in U.S. Pat. Nos. 5,686,082 and 5,686,367. Examples of flavonoids particularly suitable for use in the present disclosure are one or more flavones, one or more isoflavones, one or more coumarins, one or more chromones, one or more dicoumarols, one or more chromanones, one or more chromanols, isomers (e.g., cis/trans isomers) thereof, and mixtures thereof.

Exemplary flavonoids include flavones and isoflavones, in particular daidzein (7,4′-dihydroxy isoflavone), genistein (5,7,4′-trihydroxy isoflavone), equol (7,4′-dihydroxy isoflavan), 5,7-dihydroxy-4′-methoxy isoflavone, soy isoflavones (a mixture extracted from soy) and other plant sources of such mixtures (e.g., red clover), and mixtures thereof. Other exemplary materials include flavanones such as hesperitin, hesperidin, and mixtures thereof. Flavonoid compounds useful herein are commercially available from a number of sources, e.g., Indofine Chemical Company, Inc., Steraloids, Inc., and Aldrich Chemical Company, Inc.

In one embodiment, the composition may comprise from about 0.0001%, 0.001%, 0.01%, 0.05%, 0.1%, 0.5%, or 1% to about 30%, 25%, 20%, 10%, 7%, 5%, or 3%, by weight of the composition, of one or more flavonoid compounds.

### 8. Particulate Materials

The personal care compositions of the present disclosure can comprise one or more additional particulate materials. Nonlimiting examples of particulate materials useful in the present disclosure include colored and uncolored pigments, interference pigments, inorganic powders, organic powders, composite powders, optical brightener particles, and combinations thereof. In one embodiment, the composition may comprise from about 0.0001%, 0.001%, 0.01%, 0.05%, 0.1%, 0.5%, 1%, or 2% to about 50%, 25%, 20%, 10%, 7%, 5%, or 3% by weight of the composition, of particulate(s). There are no specific limitations as to the pigment, colorant or filler powders used in the composition.

Particulate materials useful herein can include, but are not limited to, bismuth oxychloride, sericite, mica, mica treated with barium sulfate or other materials, zeolite, kaolin, silica, boron nitride, lauroyl lysine, nylon, polyethylene, tale, styrene, polypropylene, polystyrene, ethylene/acrylic acid copolymer, polyurethane, aluminum oxide, silicone resin, barium sulfate, calcium carbonate, cellulose acetate. PTFE, polymethyl methacrylate, starch, modified starches such as aluminum starch octenyl succinate, silk, glass, and mixtures thereof. Suitable commercial examples of particulates include, but are not limited, to polymeric particles chosen from the polymethylsilsesquioxane resin microspheres such as including materials sold under the tradename Tospearl® by Momentive Performance Materials Inc., microspheres of polymethylmethacrylates such Micropearl M305 by SEPPIC, spherical particles of crosslinked polydimethylsiloxanes, especially such as those sold by Dow Corning 9506 Cosmetic Power by Dow Corning, spherical particles of polyamide and more specifically Nylon 12 such as Orgasol® 2002 line by Atochem, polystyrene microspheres such as for example those sold under the name Dynospheres® by Dyno Particles, ethylene acrylate copolymer sold under the name BA209 by Kobo. PTFE, polypropylene, aluminum starch ocetenylsuccinate such as those sold under the name Dry-Flo® by AkzoNobel, microspheres of polyethylene such as those sold under the name of Microthene® FN510-00 by Equistar and under the name Micropoly by Presperse, Inc., silicone resin, polymethylsilsesquioxane silicone polymer, and mixtures thereof. Suitable particulate materials include spherical powders with an average primary particle size of from about 0.1 to about 75 microns or from about 0.2 to about 30 microns.

Other suitable particulate materials include interference pigments. Interference pigments, for purposes of the present specification, are defined as thin platelike layered particles having two or more layers of controlled thickness with different refractive indices that yield a characteristic reflected color from the interference of typically two, but occasionally more, light reflections, from different layers of the platelike particle. The most common examples of interference pigments are micas layered with about 50-300 nm films of TiO₂, Fe₂O₃, silica, tin oxide, and/or Cr₂O₃. Such pigments are often pearlescent. Pearl pigments reflect, refract and transmit light because of the transparency of pigment particles and the large difference in the refractive index of mica platelets and, for example, the titanium dioxide coating. Useful interference pigments are available commercially from a wide variety of suppliers, for example. Rona (Timiron™ and Dichrona™), Presperse (Flonac™), Englehard (Duochrome™), Kobo (KTZ Interfine and KTZ Interval), BASF (Reflecks™) and Eckart (Prestige series). Suitable interference pigments may have a small particle sizes, with an average diameter of individual particles less than about 75 microns in the longest direction, or less than about 50 microns.

Other particulate materials include pigments which can provide color to the personal care composition. Suitable pigments include inorganic pigments, organic pigments and combinations thereof. Examples of such useful inorganic pigments include iron oxides, ferric ammonium ferrocyanide, manganese violet, ultramarine blue, and Chrome oxide. Organic pigments can include natural colorants and synthetic monomeric and polymeric colorants. An example is phthalocyanine blue and green pigment. Also useful are lakes, primary FD&C or D&C lakes and blends thereof. Also useful are encapsulated soluble or insoluble dyes and other colorants. Inorganic white or uncolored pigments useful in the present disclosure, for example TiO₂, ZnO, or ZrO₂, are commercially available from a number of sources. One example of a suitable particulate material contains the material available from U.S. Cosmetics (TRONOX TiO2 series, SAT-T CR837, a rutile TiO2). Suitable pigments include charged dispersions of titanium dioxide, as are disclosed in U.S. Pat. No. 5,997,887.

Colored or uncolored pigments may have a primary average particle size of from about 10 nm, 15 nm, or 20 nm to about 100,000 nm, 5,000 nm, or 1000 nm. Mixtures of the same or different pigments having different particle sizes are also useful herein (e.g., incorporating a TiO₂ having a primary particle size of from about 100 nm to about 400 nm with a TiO₂ having a primary particle size of from about 10 am to about 50 nm).

The particulate materials can be surface treated to provide added stability and/or for ease of formulation. Non-limiting examples of suitable coating materials include silicones, lecithin, amino acids, metal soaps, polyethylene and collagen. These surface treatments may be hydrophobic or hydrophilic. Particularly useful hydrophobic pigment treatments include polysiloxane treatments such as those disclosed in U.S. Pat. No. 5,143,722.

### 8. UV Actives (Light Protective Filters)

The compositions of the instant disclosure may contain a UV active. As used herein, a “UV active” or a “Light Protective Filter” includes both sunscreen agents and physical sunblocks. Suitable UV actives may be organic or inorganic. Suitable UV actives are listed in the functional category of “Sunscreen Agents” in the Personal Care Product Council's International Cosmetic Ingredient Dictionary and Handbook, Thirteenth Edition, 2010.

Suitable UV actives include dibenzoylmethane derivatives including 2-methyldibenzoylmethane, 4-methyldibenzoylmethane, 4-isopropyldibenzoylmethane, 4-tert-butyldibenzoylmethane, 2,4-dimethyldibenzoylmethane, 2,5-dimethyldibenzoylmethane, 4,4′-diisopropyldibenzoylmethane, 4,4′-dimethoxy dibenzoylmethane, 4-tert-butyl-4′-methoxy dibenzoylmethane (i.e., butyl methoxydibenzoylmethane or avobenzone) (commercially available as PARSOL® 1789 from DSM), 2-methyl-5-isopropyl-4′-methoxy dibenzoylmethane, 2-methyl-5-tert-butyl-4′-methoxydibenzoylmethane, 2,4-dimethyl-4′-methoxy dibenzoylmethane, and 2,6-dimethyl-4-tert-butyl-4′-methoxy dibenzoylmethane. Other suitable UV actives include 2-ethylhexyl-p-methoxycinnamate (commercially available as PARSOL® MCX from DSM), 2-hydroxy-4-methoxybenzophenone, benzonphenone-3 (i.e., oxybeznone), octyldimethyl-p-aminobenzoic acid, digalloyltrioleate, 2,2-dihydroxy-4-methoxybenzophenone, ethyl-4-(bis(hydroxy-propyl)aminobenzoate, 2-ethylhexyl-2-cyano-3,3-diphenylacrylate, 2-ethylhexyl-salicylate, glyceryl-p-aminobenzoate, 3,3,5-tri-methylcyclohexylsalicylate, methylanthranilate, p-dimethyl-aminobenzoic acid or aminobenzoate, 2-ethylhexyl-p-dimethyl-amino-benzoate, 2-phenylbenzimidazole-S-sulfonic acid, 2-(p-dimethylaminophenyl)-5-sulfonicbenzoxazoic acid, octocrylene, zinc oxide, titanium dioxide, and mixtures of these compounds.

Particularly suitable UV actives useful in the compositions of the present disclosure are 2-ethylhexyl-p-methoxycinnamate, 4-tert-butyl-4′-methoxy dibenzoylmethane, 2-hydroxy-4-methoxybenzo-phenone, 2-phenylbenzimidazole-5-sulfonic acid, octocrylene, zinc oxide, titanium dioxide, and mixtures thereof.

Other suitable UV actives include 4-methylbenzylidene camphor (commercially available as PARSOL® 5000 from DSM or Eusolex 6300 from Merck), methylene bis-benzotriazolyl tetramethylbutylphenol (i.e., bisoctrizole, commercially available as Tinosorb® M from BASF), bis-ethylhexyloxyphenol methoxyphenol triazine (i.e., bemotrizinol, commercially available as Tinosorb® S from BASF), disodium phenyl dibenzimidazole tetrasulfonate (i.e., Bisdisulizole disodium, commercially available as Neo Heliopan® AP from Symrise), Ethylhexyl triazone (commercially available as Uvinul® T 150 from BASF), Drometrizole trisiloxane (marketed as Mexoryl XL by L'Oreal), Sodium Dihydroxy Dimethoxy Disulfobenzophenone (i.e., benzophenone-9, commercially available as Uvinul® DS 49 from BASF), Diethylamino Hydroxybenzoyl Hexyl Benzoate (commercially available as Uvinul® A Plus from BASF), diethylhexyl butamido triazone (i.e., Iscotrizinol, commercially available as Uvasorb®) HEB by 3V Sigma), Polysilicone-15 (i.e., commercially available as PARSOL® SLX from DSM), and Isoamyl p-Methoxycinnamate (i.e., amiloxate, commercially available as Neo Heliopan® E 1000 from Symrise).

### 9. Photostabilizers

A suitable photostabilizer is alpha cyanodiphenylacrylate is as disclosed in U.S. Pat. No. 7,713,519. The alpha-cyanodiphenylacrylate may have the general formula:

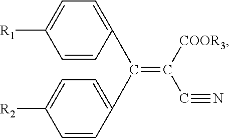

wherein one or both of R1 and R2 is independently a straight or branched chain C1-30 alkoxy radical and any non-alkoxy R1 or R2 radical is hydrogen; and R3 is a straight or branched chain C1-30 alkyl. Alternately, one or both of R1 and R2 is independently a C1-8 alkoxy radical and any non-alkoxy R1 or R2 radical is hydrogen; and R3 is a straight of branched chain C2-20 alkyl. Alternately, one or both of R1 and R2 is independently methoxy, and any non-methoxy R1 or R2 is hydrogen; and R3 is a straight or branched chain C2-20 alkyl.

A suitable alpha-cyanodiphenylacrylate is ethylhexyl methoxycrylene, or 2-ethylhexyl 2-cyano-3-(4-methoxyphenyl)-3-phenylpropenoate, wherein R1 is methoxy, R2 is hydrogen, and R3 is 2-ethylhexyl. This material is available from Hallstar Company under trade name Solastay® S 1.

Another suitable photostabilizer includes diesters or polyesters of naphthalene dicarboxylic acid as disclosed in U.S. Pat. Nos. 5,993,789, 6,113,931, 6,126,925 and 6,284,916. Suitable diesters or polyesters of naphthalene dicarboxylic acid may have the following formula:

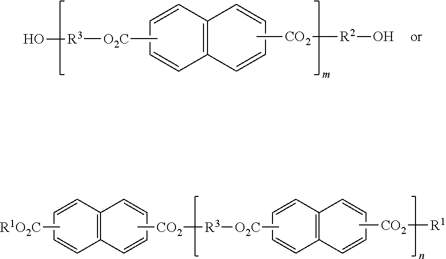

wherein each R¹ independently is an alkyl group having 1 to 22 carbon atoms, or a diol having the formula HO—R²—OH, or a polyglycol having the formula HO—R³—(—O—R₂—)ₘ—OH, and, wherein R² and R³, same or different, are each an alkylene group, straight chain or branched, having 1 to 6 carbon atoms, wherein m and n are each 1 to about 100, 1 to about 10, or 2 to about 7. A suitable diesters of naphthalene dicarboxylic acid is diethylhexyl 2,6-naphthalate available as Corapan® TQ from Symrise.

Another suitable photostabilizer is 4-hydroxybenzylidenemalonate derivatives or 4-hydroxycinnamate derivatives. Suitable materials may have the following formula:

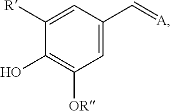

wherein A is a chromophoric group that absorbs UV-radiation, comprises one divalent group or two monovalent groups with at least one group having carbonyl (C═O) functionality; R′ is hydrogen, a linear or branched C₁-C₈ alkyl radical or a linear or branched C₁-C₈ alkoxy radical; and R″ is a linear or branched C₁-C₈ alkyl radical. Exemplary compounds include ethyl-alpha-cyano-3,5-dimethoxy-4-hydroxy cinnamate, ethyl-alpha-acetyl-3,5-dimethoxy-4-hydroxy cinnamate, iso-propyl-alpha-acetyl-3,5-dimethoxy-4-hydroxy cinnamate, iso-amyl-alpha-acetyl-3,5-dimethoxy-4-hydroxy cinnamate, 2-ethylhexyl-alpha-acetyl-3,5-dimethoxy-4-hydroxy cinnamate, diethyl-3,5-dimethoxy-4-hydroxy benzylidene malonate, di-(2-ethylhexyl)-3,5-dimethoxy-4-hydroxy benzylidene malonate, diisoamyl-3,5-dimethoxy-4-hydroxy benzylidene malonate, didodecyl-3,5-dimethoxy-4-hydroxy benzylidene malonate, dipalmitoyl-3,5-dimethoxy-4-hydroxy benzylidene malonate, and di-isopropyl-3,5-dimethoxy-4-hydroxy benzylidene malonate. A particularly suitable compound is diethylhexyl syringylidenemalonate (INCI name) available under the tradename Oxynex® ST from EMD Chemicals, Inc., having the formula:

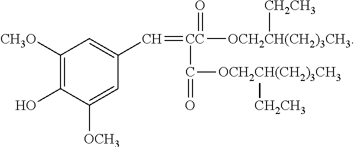

Additional suitable 4-hydroxybenzylidenemalonate derivatives or 4-hydroxycinnamate derivatives are disclosed in U.S. Pat. No. 7,357,919 and U.S. Patent Application Publication No. 2003/0108492A1 and US2003/0157035A.

Another suitable photostabilizer is a 2-pyrrolidinone-4-carboxy ester compounds. Suitable 2-pyrrolidinone-4-carboxy ester compounds may have the following formula:

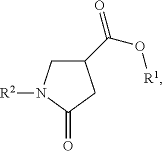

wherein R¹ is a linear or branched C₁-C₂₀ alkyl radical, and R² is a linear or branched C₁-C₂₀ alkyl radical which can contain a C₅-C₆ ring, the phenyl radical, the benzyl radical or the phenethyl radical. Exemplary radicals for R¹ and R² include methyl, ethyl, n-propyl, isopropyl, n-butyl, isobutyl, tert-butyl, n-octyl, 2-ethylhexyl, dodecyl, hexadecyl, cyclohexyl and methylcyclohexyl radicals. Particular examples of 2-pyrrolidinone-4-carboxy ester compounds are provided in U.S. Patent Application Publication No. 2010/0183529.

Other suitable photostabilizers include: silicon-containing s-triazines substituted with two aminobenzoate or aminobenzamide groups as described in U.S. Patent Application Publication No. 2008/0145324; fluorene derivatives as described in U.S. Patent Application Publications Nos. 2004/00579912, 2004/00579914, 200/00579916, and 2004/062726; piperidinol salts as described in U.S. Patent Application Publications No. 2005/0220727 including tris(tetramethylhydroxypiperidinol) citrate sold under the tradename Tinogard® Q by Ciba; and arylalkyl amides and esters as described in U.S. Patent Application Publication No. 2008/0019930.

Other suitable photostabilizers are listed in the functional category of “Light Stabilizers” in the Personal Care Product Council's International Cosmetic Ingredient Dictionary and Handbook, Thirteenth Edition, 2010.

In one embodiment, the personal care composition may comprise from about 0.0001%, 0.001%, 0.01%, 0.05%, 0.1%, 0.5%, or 1% to about 30%, 25%, 20%, 10%, 7%, or 5%, by weight of the composition, of one or more suitable photostabilizer. In certain embodiments, the personal care composition may comprise at least one photostabilizer and at least one UV active. In particular embodiments, the UV active is a dibenzoylmethane derivative. In a particular embodiment, the UV active is 4,4′-t-butyl methoxydibenzoyl-methane (i.e., avobenzone).

### 10. Anti-Oxidants/Racial Scavengers

The personal care compositions of the present disclosure can include an anti-oxidant/radical scavenger. In one embodiment, the composition may comprise from about 0.0001%, 0.0019%, 0.01%, 0.05%, 0.1%, 0.5%, or 1% to about 30%, 25%, 20%, 10%, 7%, 5%, or 39%, by weight of the composition, of one or more anti-oxidant/radical scavengers.

Suitable anti-oxidants are listed in the functional category of “Antioxidants” in the Personal Care Product Council's International Cosmetic Ingredient Dictionary and Handbook, Thirteenth Edition, 2010.

Suitable anti-oxidants include butylated hydroxytoluene (BHT) and butylated hydroxyanisole (BHA). BHT can be described by the general formula:

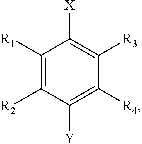

wherein X is OH or SH;
Y is selected from the group consisting of H, OH, OR₅, COOR₅, alkyl, cycloalkyl, heteroalkyl, heterocycloalkyl, aromatic, heteroaromatic, carboxamido, sulfonamido, carbamate, urea, and trialkylsilyl;
R₁, R₂, R₃, R₄ are selected from the group consisting of alkyl, cycloalkyl, heteroalkyl, heterocycloalkyl, aromatic, heteroaromatic, OR₅, carboxamido, sulfonamido, formyl, acyl, carboxyl, carboxylate, carbamate, urea, trialkylsilyl, hydroxyl, and hydrogen;
R₅ is selected from the group consisting of alkyl, cycloalkyl, heteroalkyl, heterocycloalkyl, aromatic, heteroaromatic, trialkylsilyl, acyl, and hydrogen.

Other anti-oxidants/radical scavengers such as ascorbic acid (vitamin C), tocopherol (vitamin E), tocopherol sorbate, tocopherol acetate, other esters of tocopherol, 6-hydroxy-2,5,7,8-tetramethylchroman-2-carboxylic acid (commercially available under the tradename Trolox®), amines (e.g., N,N-diethylhydroxylamine, amino-guanidine), nordihydroguaiaretic acid, bioflavonoids, amino acids, silymarin, sorbic acids and its salts, lipoic acid, olive extracts, green tea extracts, white tea extracts, black tea extracts, polyphenols such as proanthocyanidine from pine bark, carotenoids, curcumin compounds such as tetrahydrocurcumin, OCTA (L-2-oxo-4-thiazolidine carboxylic acid), glutathione, and grape skin/seed extracts may be used. Suitable anti-oxidants/radical scavengers can be selected from esters of tocopherol such as tocopherol acetate.

In one embodiment, the personal care composition comprises tocopherol sorbate. As used herein, “tocopherol sorbate” refers to the sorbic acid ester of tocopherol, a detailed description of which can be found in issued U.S. Pat. No. 5,922,758. In one embodiment, the composition may comprise from about 0.0001%, 0.001%, 0.01%, 0.05%, 0.1%, 0.5%, or 1% to about 50%, 25%, 20%, 10%, 7%, or 5%, by weight of the composition, of the tocopherol sorbate.

### 11. Botanical Extracts

The personal care compositions of the instant disclosure may comprise botanical extracts. In one embodiment, the composition may comprises from about 0.0001%, 0.0005% 0.001%, 0.01%, 0.05%, 0.1%, 0.5%, or 1% to about 30%, 25%, 20%, 10%, 7%, 5%, 3%, by weight of the composition, of one or more botanical extracts. Suitable botanical extracts include extracts from plants (herbs, roots, flowers, fruits, seeds) such as flowers, fruits, vegetables, and so on, including yeast ferment extract, Patina pavonica extract, Thermus thermophilis ferment extract. Camelina sativa seed oil, Boswellia serrata extract, olive extract, Bodopsis thaliana extract, Acacia Dealbata extract, Acer Saccharinum (sugar maple), acidopholus, acorus, aesculus, Alicaligenes polysaccharides, agaricus, agave, agrimonia, algae, aloe, citrus, brassica, cinnamon, orange, apple, blueberry, cranberry, peach, pear, lemon, lime, pea, seaweed, caffeine, green tea, chamomile, willowbark, mulberry, poppy, and the like. Further specific examples include, but are not limited to, Glycyrrhiza glabra, Salix nigra, Macrocyestis pyrifera, Pyrus malus, Saxifraga sarmentosa, Vitis vinifera, Morus nigra, Scutellaria baicalensis, Anthemis nobilis, Salvia sclarea, Rosmarinus officianalis, Citrus medica limonion, Ginkgo biloba Panax ginseng, Siegesbeckia orientalis, Fructus mame, Ascophyllum nodosum, Bifida Ferment lysate, Glycine soja extract, Beta vulgaris, Haberlea rhodopensis, Polygonum cuspidatum, Citrus Aurantium dulcis, Vitis vinifera, Selaginella tamariscina, Humulus lupulus, Citrus reticulata Peel, Punica granatuni, Asparagopsis, Curcuma longa, Menyanthes trifoliata, Helianthus annuus, Hordeum vidgare, Cucumis sativus, Evernia prunastri, Evernia furfuracea, Laminaria angustata, Laminaria cloustoni, Laminaria digitata, Laminaria digitata, Laminaria hyperborea, Laminaria japonica, Laminaria longissima, Laminaria ochotensis, Laminaria ochroleuca, Laminaria saccharina, and mixtures thereof. Other suitable actives are listed in the functional category of “Biological Products” in the Personal Care Product Council's International Cosmetic Ingredient Dictionary and Handbook, Thirteenth Edition, 2010.

Any other suitable optional component can also be included in the personal care composition of the present disclosure, such as those ingredients that are conventionally used in given product types. The Personal Care Product Council's International Cosmetic Ingredient Dictionary and Handbook, Thirteenth Edition, 2010, describes a wide variety of nonlimiting functional materials that can be added to the composition herein. Examples of these functional classes include, but are not limited to: abrasives, absorbents, fragrances, anti-acne agents, anti-caking agents, antifoaming agents, antimicrobial agents (e.g., iodopropyl butylcarbamate), antifungal agents, antioxidants, binders, buffering agents, bulking agents, chelating agents, colorants, cosmetic astringents, cosmetic biocides, denaturants, drug astringents, external analgesics, film formers, opacifying agents, pH adjusters, plant derivatives, plant extracts, plant tissue extracts, plant seed extracts, plant oils, botanicals, botanical extracts, preservatives, propellants, reducing agents, sebum control agents, sequestrants, skin bleaching agents, skin-conditioning agents (e.g. humectants and occlusive agents), and skin protectants. Other suitable optional person care ingredients include materials listed in paragraphs 513-839 of U.S Patent Application No. 2010/0112100.

### Methods for Treating Sensitive Skin

Another aspect of the disclosure is directed to a method for reducing or slowing down at least one sign of aging in a subject with sensitive skin, comprising administering the subject a personal care composition described herein. In some embodiments, the administration of the personal care composition results in a reduction in wrinkles, or a delay in the onset of wrinkles.

Another aspect of the disclosure is directed to a method for treating sensitive skin in a subject comprising administering the subject a personal care composition described herein. In some embodiments, the sensitive skin exhibits at least one unpleasant sensation before the treatment, the at least one unpleasant sensation selected from the group consisting of tingling, heat, burning, itching, tightness and erythema.

In some embodiments, the personal care composition comprises niacinamide, Edelweiss (Leontopodium alpinum) extract, rice (Oryza sativa) lees concentrate, panthenol and soy-derived glycopeptides.

In some embodiments, the subject comprises a human. In some embodiments, the subject has been exposed to pollution. In some embodiments, the subject has been exposed to air pollution. In some embodiments, the subject has been exposed to air pollution having an Air Quality Index (AQI) of above 100. In some embodiments, the subject is exposed to air pollution having an Air Quality Index (AQI) of above 150. In some embodiments, the subject is exposed to air pollution having an Air Quality Index (AQI) of above 200. In some embodiments, the subject is exposed to air pollution having an Air Quality Index (AQI) of above 250. In some embodiments, the subject is exposed to air pollution having an Air Quality Index (AQI) of above 300.

In some embodiments, the personal care composition comprises, by weight: (a) 0.1%-10% (e.g., 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.7%, 2.8%, 2.9%, 3%, 3.1%, 3.2%, 3.3%, 3.4%, 3.5%, 3.6%, 3.7%, 3.8%, 3.9%, 4%, 4.1%, 4.2%, 4.3%, 4.4%, 4.5%, 4.6%, 4.7%, 4.8%, 4.9%, 5%, 5.1%, 5.2%, 5.3%, 5.4%, 5.5%, 5.6%, 5.7%, 5.8%, 5.9%, 6%, 6.1%, 6.2%, 6.3%, 6.4%, 6.5%, 6.6%, 6.7%, 6.8%, 6.9%, 7%, 7.1%, 7.2%, 7.3%, 7.4%, 7.5%, 7.6%, 7.7%, 7.8%, 7.9%, 8%, 8.1%, 8.2%, 8.3%, 8.4%, 8.5%, 8.6%, 8.7%, 8.8%, 8.9%, 9%, 9.1%, 9.2%, 9.3%, 9.4%, 9.5%, 9.6%, 9.7%, 9.8%, 9.9%, 10%) niacinamide, (b) 0.0001%-10% (e.g., 0.001%, 0.002%, 0.005%, 0.008%, 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.7%, 2.8%, 2.9%, 3%, 3.1%, 3.2%, 3.3%, 3.4%, 3.5%, 3.6%, 3.7%, 3.8%, 3.9%, 4%, 4.1%, 4.2%, 4.3%, 4.4%, 4.5%, 4.6%, 4.7%, 4.8%, 4.9%, 5%, 5.1%, 5.2%, 5.3%, 5.4%, 5.5%, 5.6%, 5.7%, 5.8%, 5.9%, 6%, 6.1%, 6.2%, 6.3%, 6.4%, 6.5%, 6.6%, 6.7%, 6.8%, 6.9%, 7%, 7.1%, 7.2%, 7.3%, 7.4%, 7.5%, 7.6%, 7.7%, 7.8%, 7.9%, 8%, 8.1%, 8.2%, 8.3%, 8.4%, 8.5%, 8.6%, 8.7%, 8.8%, 8.9%, 9%, 9.1%, 9.2%, 9.3%, 9.4%, 9.5%, 9.6%, 9.7%, 9.8%, 9.9%, 10%) Edelweiss (Leontopodium alpinum) extract, (c) 0.001%-10% (e.g., 0.001%, 0.002%, 0.003%, 0.004%, 0.005%, 0.006%, 0.007%, 0.008%, 0.009%, 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.7%, 2.8%, 2.9%, 3%, 3.1%, 3.2%, 3.3%, 3.4%, 3.5%, 3.6%, 3.7%, 3.8%, 3.9%, 4%, 4.1%, 4.2%, 4.3%, 4.4%, 4.5%, 4.6%, 4.7%, 4.8%, 4.9%, 5%, 5.1%, 5.2%, 5.3%, 5.4%, 5.5%, 5.6%, 5.7%, 5.8%, 5.9%, 6%, 6.1%, 6.2%, 6.3%, 6.4%, 6.5%, 6.6%, 6.7%, 6.8%, 6.9%, 7%, 7.1%, 7.2%, 7.3%, 7.4%, 7.5%, 7.6%, 7.7%, 7.8%, 7.9%, 8%, 8.1%, 8.2%, 8.3%, 8.4%, 8.5%, 8.6%, 8.7%, 8.8%, 8.9%, 9%, 9.1%, 9.2%, 9.3%, 9.4%, 9.5%, 9.6%, 9.7%, 9.8%, 9.9%, 10%) rice (Oryza sativa) lees concentrate, (d) 0.1%-10% (e.g., 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.7%, 2.8%, 2.9%, 3%, 3.1%, 3.2%, 3.3%, 3.4%, 3.5%, 3.6%, 3.7%, 3.8%, 3.9%, 4%, 4.1%, 4.2%, 4.3%, 4.4%, 4.5%, 4.6%, 4.7%, 4.8%, 4.9%, 5%, 5.1%, 5.2%, 5.3%, 5.4%, 5.5%, 5.6%, 5.7%, 5.8%, 5.9%, 6%, 6.1%, 6.2%, 6.3%, 6.4%, 6.5%, 6.6%, 6.7%, 6.8%, 6.9%, 7%, 7.1%, 7.2%, 7.3%, 7.4%, 7.5%, 7.6%, 7.7%, 7.8%, 7.9%, 8%, 8.1%, 8.2%, 8.3%, 8.4%, 8.5%, 8.6%, 8.7%, 8.8%, 8.9%, 9%, 9.1%, 9.2%, 9.3%, 9.4%, 9.5%, 9.6%, 9.7%, 9.8%, 9.9%, 10%) panthenol, and (e) 0.001%-5% (e.g., 0.001%, 0.002%, 0.003%, 0.004%, 0.005%, 0.006%, 0.007%, 0.008%, 0.009%, 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.7%, 2.8%, 2.9%, 3%, 3.1%, 3.2%, 3.3%, 3.4%, 3.5%, 3.6%, 3.7%, 3.8%, 3.9%, 4%, 4.1%, 4.2%, 4.3%, 4.4%, 4.5%, 4.6%, 4.7%, 4.8%, 4.9%, 5%) soy-derived glycopeptides.

In some embodiments, the personal care composition comprises, by weight: (a) 0.1%-4% (e.g., 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.7%, 2.8%, 2.9%, 3%, 3.1%, 3.2%, 3.3%, 3.4%, 3.5%, 3.6%, 3.7%, 3.8%, 3.9%, 4%,) niacinamide, (b) 0.0001%-4% (e.g., 0.001%, 0.002%, 0.005%, 0.008%, 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.7%, 2.8%, 2.9%, 3%, 3.1%, 3.2%, 3.3%, 3.4%, 3.5%, 3.6%, 3.7%, 3.8%, 3.9%, 4%) Edelweiss (Leontopodium alpinum) extract, (c) 0.001%-2% (e.g., 0.001%, 0.002%, 0.003%, 0.004%, 0.005%, 0.006%, 0.007%, 0.008%, 0.009%, 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%) rice (Oryza sativa) lees concentrate, (d) 0.1%-3% (e.g., 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.7%, 2.8%, 2.9%, 3%) panthenol, and (e) 0.002%-4% (e.g., 0.002%, 0.003%, 0.004%, 0.005%, 0.006%, 0.007%, 0.008%, 0.009%, 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.7%, 2.8%, 2.9%, 3%, 3.1%, 3.2%, 3.3%, 3.4%, 3.5%, 3.6%, 3.7%, 3.8%, 3.9%, 4%) soy-derived glycopeptides.

In some embodiments, the personal care composition comprises, by weight: (a) 0.1%-10% (e.g., 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.7%, 2.8%, 2.9%, 3%, 3.1%, 3.2%, 3.3%, 3.4%, 3.5%, 3.6%, 3.7%, 3.8%, 3.9%, 4%, 4.1%, 4.2%, 4.3%, 4.4%, 4.5%, 4.6%, 4.7%, 4.8%, 4.9%, 5%, 5.1%, 5.2%, 5.3%, 5.4%, 5.5%, 5.6%, 5.7%, 5.8%, 5.9%, 6%, 6.1%, 6.2%, 6.3%, 6.4%, 6.5%, 6.6%, 6.7%, 6.8%, 6.9%, 7%, 7.1%, 7.2%, 7.3%, 7.4%, 7.5%, 7.6%, 7.7%, 7.8%, 7.9%, 8%, 8.1%, 8.2%, 8.3%, 8.4%, 8.5%, 8.6%, 8.7%, 8.8%, 8.9%, 9%, 9.1%, 9.2%, 9.3%, 9.4%, 9.5%, 9.6%, 9.7%, 9.8%, 9.9%, 10%) niacinamide, (b) 0.0001%-10% (e.g., 0.001%, 0.002%, 0.005%, 0.008%, 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.7%, 2.8%, 2.9%, 3%, 3.1%, 3.2%, 3.3%, 3.4%, 3.5%, 3.6%, 3.7%, 3.8%, 3.9%, 4%, 4.1%, 4.2%, 4.3%, 4.4%, 4.5%, 4.6%, 4.7%, 4.8%, 4.9%, 5%, 5.1%, 5.2%, 5.3%, 5.4%, 5.5%, 5.6%, 5.7%, 5.8%, 5.9%, 6%, 6.1%, 6.2%, 6.3%, 6.4%, 6.5%, 6.6%, 6.7%, 6.8%, 6.9%, 7%, 7.1%, 7.2%, 7.3%, 7.4%, 7.5%, 7.6%, 7.7%, 7.8%, 7.9%, 8%, 8.1%, 8.2%, 8.3%, 8.4%, 8.5%, 8.6%, 8.7%, 8.8%, 8.9%, 9%, 9.1%, 9.2%, 9.3%, 9.4%, 9.5%, 9.6%, 9.7%, 9.8%, 9.9%, 10%) Edelweiss (Leontopodium alpinum) extract, (c) 0.001%-10% (e.g., 0.001%, 0.002%, 0.003%, 0.004%, 0.005%, 0.006%, 0.007%, 0.008%, 0.009%, 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.7%, 2.8%, 2.9%, 3%, 3.1%, 3.2%, 3.3%, 3.4%, 3.5%, 3.6%, 3.7%, 3.8%, 3.9%, 4%, 4.1%, 4.2%, 4.3%, 4.4%, 4.5%, 4.6%, 4.7%, 4.8%, 4.9%, 5%, 5.1%, 5.2%, 5.3%, 5.4%, 5.5%, 5.6%, 5.7%, 5.8%, 5.9%, 6%, 6.1%, 6.2%, 6.3%, 6.4%, 6.5%, 6.6%, 6.7%, 6.8%, 6.9%, 7%, 7.1%, 7.2%, 7.3%, 7.4%, 7.5%, 7.6%, 7.7%, 7.8%, 7.9%, 8%, 8.1%, 8.2%, 8.3%, 8.4%, 8.5%, 8.6%, 8.7%, 8.8%, 8.9%, 9%, 9.1%, 9.2%, 9.3%, 9.4%, 9.5%, 9.6%, 9.7%, 9.8%, 9.9%, 10%) rice (Oryza sativa) lees concentrate, and (d) 0.1%-10% (e.g., 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.7%, 2.8%, 2.9%, 3%, 3.1%, 3.2%, 3.3%, 3.4%, 3.5%, 3.6%, 3.7%, 3.8%, 3.9%, 4%, 4.1%, 4.2%, 4.3%, 4.4%, 4.5%, 4.6%, 4.7%, 4.8%, 4.9%, 5%, 5.1%, 5.2%, 5.3%, 5.4%, 5.5%, 5.6%, 5.7%, 5.8%, 5.9%, 6%, 6.1%, 6.2%, 6.3%, 6.4%, 6.5%, 6.6%, 6.7%, 6.8%, 6.9%, 7%, 7.1%, 7.2%, 7.3%, 7.4%, 7.5%, 7.6%, 7.7%, 7.8%, 7.9%, 8%, 8.1%, 8.2%, 8.3%, 8.4%, 8.5%, 8.6%, 8.7%, 8.8%, 8.9%, 9%, 9.1%, 9.2%, 9.3%, 9.4%, 9.5%, 9.6%, 9.7%, 9.8%, 9.9%, 10%) panthenol.

In some embodiments, “niacinamide” comprises the formula:

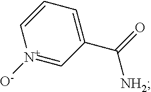

derivatives thereof; and salts of any of the foregoing.

In some embodiments, the personal care composition comprises 0.1%-2% (e.g., 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%) niacinamide by weight. In some embodiments, the personal care composition comprises 0.5%-1.5% (e.g., 0.5%, 0.6%, 0.7%, 0.8%, 0.9%, 1%, 1.1%, 1.2%, 1.3%, 1.4%, 1.5%) niacinamide by weight.

In some embodiments, the Edelweiss (Leontopodium alpinum) extract comprises Chlorogenic acid, Chlorogenic acid derivatives, Luteoline-7-O-glucoside, Luteoline-4′-O-glucoside, Apigenine-7-O-glucoside, luteolin, and Leontopodic Acid as bioactive agents. In a specific embodiment the Edelweiss (Leontopodium alpinum) extract comprises Alpaflor® Edelweiss extract.

In some embodiments, the personal care composition comprises 0.1%-2% (e.g., 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%) Edelweiss (Leontopodium alpinum) extract by weight. In some embodiments, the personal care composition comprises 0.5%-1.5% (e.g., 0.5%, 0.6%, 0.7%, 0.8%, 0.9%, 1%, 1.1%, 1.2%, 1.3%, 1.4%, 1.5%) Edelweiss (Leontopodium alpinum) extract by weight.

In some embodiments, the rice (Oryza sativa) lees concentrate comprises amino acids, oleic acid, linolenic acid, phytic acid, palmitic acid and vitamin B derivatives as bioactive agents. In some embodiments, the rice (Oryza sativa) lees concentrate has a pH between 4.0 and 7.0. In some embodiments, the rice (Oryza sativa) lees concentrate has a specific gravity (d20/20) between 0.99 and 1.05.

In some embodiments, the personal care composition comprises 0.1%-2% (e.g., 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%) rice (Oryza sativa) lees concentrate by weight. In some embodiments, the personal care composition comprises 0.5%-1.5% (e.g., 0.5%, 0.6%, 0.7%, 0.8%, 0.9%, 1%, 1.1%, 1.2%, 1.3%, 1.4%, 1.5%) rice (Oryza sativa) lees concentrate by weight.

In some embodiments, “panthenol” comprises the formula:

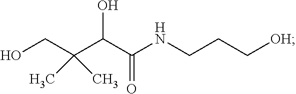

derivatives thereof; and salts of any of the foregoing.

In some embodiments, the personal care composition comprises 0.1%-2% (e.g., 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%) panthenol by weight. In some embodiments, the personal care composition comprises 0.5%-1.5% (e.g., 0.5%, 0.6%, 0.7%, 0.8%, 0.9%, 1%, 1.1%, 1.2%, 1.3%, 1.4%, 1.5%) panthenol by weight.

In some embodiments, the soy-derived glycopeptides are glycopeptides that are derived from highly purified soya fiber. In some embodiments, the soy-derived glycopeptides may be blended with stabilizers and preservatives. An exemplary soy-derived glycopeptide is sold under the trade name PRO-COLL-ONE+® by Silab.

In some embodiments, the personal care composition does not comprise any retinoid or retinoid derivatives.

In some embodiments, the personal care composition further comprises at least one of the following: water, anti-dehydration agent, light protective filter, antioxidant, preservative, gelling agent, solvent, pH balancing agent, and surfactant.

In some embodiments, the anti-dehydration agent comprises at least one of xylitylglucoside, anhydroxylitol, and xylitol.

In some embodiments, the light protective filter comprises at least one of zinc oxide, titanium dioxide and iron oxide.

In some embodiments, the personal care composition further comprises at least one of the following: hydrolyzed hyaluronic acid, glycerin, sodium hydroxide, phenoxyethanol, xantham gum, sodium polyacryloyldimethyl taurate, undecane, tridecane, tocopherol, dimethicone, titanium dioxide, tin oxide, and calcium aluminum borosilicate.

In some embodiments, the personal care composition comprises at least one of the following: hydrolyzed hyaluronic acid (0.001%-0.05% by weight), glycerin (0.1%-7% by weight), sodium hydroxide (0.001%-0.1% by weight), phenoxyethanol (0.1%-1% by weight), xantham gum (0.05%-0.5% by weight), sodium polyacryloyldimethyl taurate (0.1%-1.2% by weight), an emollient composition comprising undecane, tridecane, and tocopherol (0.05%-0.5% by weight), dimethicone (0.5%-5% by weight), a pigment composition comprising titanium dioxide, tin oxide, and calcium aluminum borosilicate (0.005%-0.05% by weight).

In some embodiments, the emollient composition comprises Cetiol Ultimate™. In some embodiments, the pigment composition comprises Syncrystal Gold™.

In some embodiments, the personal care composition further comprises an exfoliator. In some embodiments, the exfoliator is jojoba beads containing vitamin E.

In some embodiments, the personal care composition is a cream, a lotion, a gel, a mask, or a spray.

In some embodiments, the sensitive skin exhibits at least one unpleasant sensation before the treatment, the at least one unpleasant sensation selected from the group consisting of tingling, heat, burning, itching, tightness and erythema.

In some embodiments, the administration of the personal care composition results in a reduction in wrinkles, or a delay in the onset of wrinkles.

In some embodiments, the administration of the personal care composition reduces the signs of ageing by at least 5%, 10%, 15%, 20%, 25%, 30%, 35%, 40%, 50%, 60%, 70%, 80%, 90%, 95%, 99% or more as compared to administration of the same compound without niacinamide, Edelweiss (Leontopodium alpinum) extract, rice (Oryza sativa) lees concentrate, panthenol and soy-derived glycopeptides.

In some embodiments, the personal care composition is administered topically. In some embodiments, the personal care composition is administered every hour, every 2 hours, every 3 hours, every 6 hours, every 12 hours, or every day, every other day, or every three days. Preferably, the composition is administered once, twice, or three times, daily. In some embodiments, the personal care composition is administered for at least 10, 11, 12, 13, 14, 15, 16, 17, 18, 19 or 20 days before a reduction in signs of aging of sensitive skin (reduction of wrinkles, e.g., in the crow's feet area, increased) or at least one unpleasant sensation of sensitive skin (e.g., tingling, heat, burning, itching, tightness and erythema) is observed.

The disclosure now being generally described, it will be more readily understood by reference to the following example which is included merely for purposes of illustration of certain aspects and embodiments of the present disclosure, and are not intended to limit the disclosure.

### EXAMPLES

### Example 1: In Vitro and In Vivo Tests

The objective of this study was to evaluate in vitro and in vivo effects of the personal care compositions described herein. In some experiments, the personal care compositions comprised niacinamide, Edelweiss (Leontopodium alpinum) extract, rice (Oryza sativa) lees concentrate, and panthenol as active ingredients. Personal care compositions formulated for sensitive skin in healthy aging as described herein were compared to a retinol-containing serum. Precisely, the inventors compared pro-collagen and pro-elastic expression, inflammatory response, and clinical outcomes of aging of between the personal care compositions described herein to retinol serum.

### Material and Methods

In vitro tests were performed on Normal Human Dermal Fibroblast (NHDF) in culture with the addition of test compositions containing the same key active ingredients as described above but not containing retinol, and a retinol serum used as a comparison. Collagen, elastin, IL-8 and PGE-2 expression was measured 48 h (aging markers) or 72 h (inflammatory markers) after treatment.

In vivo tests evaluated the following signs of aging: surface roughness parameters and wrinkles (crow's feet) after 56 days of twice daily application of Composition 3, Composition 4, or the retinol serum.

### Results

An important sign of healthy skin aging is the presence of pro-collagen and elastin, whose expression of both mRNA and protein was highly induced by the composition comprising the active ingredients, whereas no effect was observed by each active ingredient alone.

Signs of skin inflammation were observed with the use of the retinol serum, as measured by induced expression of IL-8 (+582%, p<0.001 compared to the control) and PGE-2 (+104%, p<0.01). This negative inflammatory signature was not observed with the instant composition, which reduced IL-8 (−97%, p<0.05) and PGE2 (−69%, p<0.01) expression, nor with the face serum, which reduced IL-8 expression (−81%, p<0.05) and PGE2 (not significant).

The key active ingredient present in the instant compositions showed anti-wrinkles properties, as measured by a significant reduction of parameter Sa by 9.8% (p=0.0014), smothered skin relief of the crow's feet (78%, p=0.0761), and significant reduction of wrinkles by decreasing negative volume by 21.1% (p=0.001) and positive volume by 27% (p=0.0014).

### Conclusion

The results showed that the instant personal care compositions show signature similar to that of retinol, but without the risk of increased skin sensitivity, as anti-inflammatory effects were recorded in both the face and the eye serum. Particularly good performance of the eye serum was observed. Overall, the eye serum and face serum developed for sensitive skin healthy aging, act on the signs of aging while avoiding the skin irritation often observed with retinol.

### Example 2: Clinical Studies—General Results

The safety and efficacy of an embodiment of the personal care compositions described herein comprising niacinamide, Edelweiss (Leontopodium alpinum) extract, rice (Oryza sativa) lees concentrate, panthenol and highly purified soy-based glycoproteins (Composition A) as active ingredients was evaluated in during a multi centric clinical study on 2 panels of subjects: 44 Caucasian and 46 Asian women, who applied a face cream comprising the composition twice daily to the entire face for 8 and 12 weeks, respectively.

TABLE 1
Exemplary compositions used in the clinical studies.

| | Composition | Composition | Composition | Composition | Composition |
|---|---|---|---|---|---|
| Active | 1 | 2 | 3 | 4 | 5 |
| ingredients | % | % | % | % | % |
| Panthenol - | 2.5 | 2.5 | 2.5 | 2.5 | 2.5 |
| Pro Vitamin B5 | | | | | |
| Edelweiss | 0.01 | 3 | 0.5 | 3 | 3 |
| extract (manuf. | | | | | |
| solution) | | | | | |
| Rice Lees | 1 | 1 | 1 | 1 | 1 |
| Concentrate | | | | | |
| (manuf. | | | | | |
| solution) | | | | | |
| Soy-based | 0 | 0 | 2 | 2 | 0 |
| Glycopeptides | | | | | |
| (manuf. | | | | | |
| solution) | | | | | |
| Niacinamide - | 0 | 1.5 | 1.5 | 1.5 | 1.5 |
| Vitamin B3 | | | | | |

Safety was assessed by patch test, human repeat insult patch test (HRIPT), photosensitization, non-comedogenicity study, and in use multicentric study for 2 months. Efficacy was assessed by clinical scoring by expert graders during in use multicentric study for 2 or 3 months, instrumentation analysis (skin relief analysis during in use study), hydration and Transepidermal water loss (TEWL) measurements, antioxidant effect), and self-assessment questionnaires.

### Results

Hydration and TEWL analysis showed a moisturizing effect, with a significant improvement in hydration just after application (+68% at 1 h vs. control, p<0.05) that was maintained up to 24 h (+12%, p<0.05), and a significant decrease in TEWL measurement after 1 h, showing a protective effect against water loss. Consequently, the compositions described herein provide immediate and long-lasting hydration and an immediate skin barrier protection.

Application of Compositions 1-5 offered significant antioxidant properties (Calculated protection index 36.1%, p<0.05). Indeed, there was a statistically significant higher amount of squalene concentration in the treated and exposed area compared to the non-treated exposed area (+48%, p<0.05).

Dermatologist-controlled self-assessment questionnaires demonstrated a favorable effect of Compositions 1-5 on sensitive skin. A significant decrease of the global sensiscale score was observed after 4 weeks of face serum application in both panels (Asian −83%, Caucasian −66%, p<0.05), which helped to decrease skin sensitivity, which was more comfortable, less reactive, and healthier looking.

In-use test clinical scoring showed an increase in skin luminosity, radiance, homogeneity, texture, firmness, and elasticity, as well as in the appearance of wrinkles and fine lines. Wrinkle imaging and relief analysis showed a significant decrease in the total surface of wrinkles after 4 weeks of daily face serum application (−23% vs. baseline, p<0.05), total wrinkles length (−16%, p<0.05) and total depth (−15%, p<0.05).

The self-assessment questionnaire to evaluate satisfaction with Compositions 1-5 showed a high level of appreciation by volunteers in terms of skin hydration, radiance and fine lines and wrinkles, as well as effects on skin firmness, suppleness and elasticity, and fatigue, by both volunteer panels.

### Conclusions

As sensitive skin experiences accelerated aging compared to normal skin, the inventors tested safe and tolerated personal care compositions as described herein suitable for healthy sensitive skin aging. The blend of the five ingredients present in the personal care compositions provided clinical benefits to sensitive skin in terms of long-lasting hydration, protection of the skin barrier, antioxidant properties, and favorable effects on sensitive skin. Notably, the skin was more radiant and elastic, showed less and shorter wrinkles, and had a high level of appreciation.

### Example 3: Clinical Evaluations—Details

The following clinical tests were performed with the product of the instant disclosure comprising panthenol, Edelweiss extract and rice lees concentrate as active ingredients: Single patch test, HRIPT, Phototoxicity, Photosensitisation, In use test Under Dermatological and Ophthalmological controls+efficacy—Asian Panel, In use test Under Dermatological control+efficacy—Caucasian Panel, Hydration and TEWL (short term effect), Non-comedogenicity study, In vivo Anti-oxidant test, Deep cleansing/Anti pollution test, make up remover test (for the cleanser only) and Barrier Recovery Study. The products were tested in the form of a cleansing water, a night cream, a facial serum, an eye serum, and a day cream.

### Single Patch Test 48 Hours

Subject population: healthy, Number of subjects: 18 [14 females-4 males], Average age of subjects: 31 [18-45 years old].

Method: Single 48-hour topical application under occlusive patch on the upper back.

Evaluation criteria: Mean Irritation Index evaluated by a Dermatologist. MCII=0.06—Judged as Good.

Results: No irritation reaction was observed under the study conditions.

### HRIPT (Human Repeated Insult Patch Test)

Subject population: healthy, Number of subjects: 97 [79 females-18 males], Average age: 49.2 [19-70 years old].

Method: Repeated 48-hour topical application under occlusive patch on the upper back.

Evaluation criteria: Evaluation of the Mean Irritation Index and delayed contact sensitization by visual evaluation by a dermatologist.

Results: No primary or cumulative irritation nor any sensitization reaction recorded under the study conditions.

### Phototoxicity and Photosensitization

Subject population: healthy, Number of subjects: 27 [15 females-12 males], Average age: 44.3 [22-64 years old].

Method: Single and repeated topical application under occlusive patch on the upper back followed by solar exposures (U.V.-B & A and U.V.-A alone).

Evaluation criteria: Evaluation of the Mean Irritation Index and delayed contact sensitization by visual evaluation by a dermatologist.

Results: No photo-toxicity or photo-sensitization reactions were observed under the study conditions.

### In Use Test Under Normal Dermatological and Ophthalmological Control

Subjects: Subject population: healthy, with all skin types—2 panels Asian and Caucasian. Number of subjects: 44 (France), mean age 41.7 (25 to 58 yo) 43 (China), mean age 43.8 (26 to 54 yo) 100% of the subjects had sensitive skin (inclusion with a specific questionnaire), Phototype: France: I (2%), II (41%), III (57%); China: III (70%), IV (28%), V (2%). Subjects were living in a polluted environment or urban landscape. Subjects were with uneven complexion.

Application modalities: twice a day on average for 4 weeks on the whole face, under normal conditions of use.

### Evaluation:

Tolerance: Assessment of the cutaneous and ocular/periocular acceptability by the Dermatologist and Ophthalmologist*, on D0 and D28 (*Asian panel only).

Efficacy: Clinical evaluation (Scoring) by the Dermatologist of criteria of: skin radiance, skin homogeneity, skin luminosity, skin texture, skin firmness, skin elasticity and skin suppleness, on D0, D0Timm, D14 and D28. Photographs were taken using a Colorface device on D0, D0Timm, D14 and D28. Sensiscale evaluation (Misery Questionnaire) was performed on D0, D14 and D28. Healthy aging (HA) index was calculated (global score: “yes” answers on 10 questions) on D0 and D28. Self-assessment questionnaires were filled in by the subjects on D0 immediately after the first application, on D14 and D28. Skin sensitivity questionnaire was filled out in the presence of the Dermatologist on D28.

### Conclusion:

Tolerance: (China, n=42-France, n=44). The ocular, peri-ocular and coutaneous acceptability were judged, on the whole, good, after repeated applications under normal conditions of use twice a day, for 4 consecutive weeks, to the whole face, by 42 healthy Asian subjects.

The cutaneous acceptability was, on the whole, judged as good, after repeated applications under normal conditions of use, twice a day, for 4 consecutive weeks, to the whole face, by 44 healthy female (Caucasian) adult subjects. The participants reported improvement on skin radiance, homogeneity/evenness, luminosity, texture, firmness, elasticity, and suppleness. The Sensiscale D28 questionnaire found that skin irritation, tautness/tightness, general discomfort and redness were all significantly reduced (−66% overall in Caucasian and −94% in Asian subjects).

### Healthy Index

Healthy Index is a sum of ten specific prompts to evaluate skin condition. For each question 10 is the best score (“I completely agree”) and 0 is the worst (“I completely disagree”). The ten prompts in the Healthy Index were:
1. My skin appears healthy.
2. I have the control of my sensitive skin.
3. Both my skin aging and my skin sensitivity are under control.
4. I have a beautiful natural look.
5. My skin makes me looking rested.
6. My skin makes me looking radiant.
7. My skin makes me looking healthy.
8. My skin makes me feel more confident.
9. I feel I can age gracefully.
10. I feel confident in my sensitive skin in spite of harsh conditions (sun, cold, stress, pollution).

The Healthy Index questionnaire was administered to subjects on Day 0 and Day 28 of the clinical test. The subjects reported significant improvement on Healthy Index on D28 (Caucasian subjects 75%, Asian subjects 69% improvement over DO).

### Skin Sensitivity Assay

In the skin sensitivity assay (a self-assessment controlled by a Dermatologist after 28 days of use), the subjects reported that the product helped to decrease skin sensitivity (95% of the Asian panel and 71% of the Caucasian panel), and the skin was more comfortable (100% of the Asian panel and 88% of the Caucasian panel) and looked healthier (95% of the Asian panel and 76% of the Caucasian panel) after 28 days of use.

### Non-Comedogenicity Study

Subject population: healthy, prone to acne skin on the face. Subjects were with mixed oily to oily skin on the face and 25% of the subjects had a history of atopy. Number of subjects: 40 females, Average age: 30.3, [19-40 years old].

Method: Repeated applications on the face under normal conditions of use, twice a day, for 4 consecutive weeks.

Evaluation criteria: Clinical evaluation cutaneous acceptability on the face and counting of the acne-like lesions by the dermatologist.

Results: Cutaneous acceptability judged as very good and non-comedogenic potential under the study conditions.

### Hydration—TEWL Study

Subject population: healthy with a dry skin (AU<50). Number of subjects: 20 females. Average age: 58 y.o. [30-70].

Method: Single standardized application on the forearms then evaluation of the Moisturizing effect and the Trans Epidermal Water Loss (TEWL), T1H, T4H, T8H and T24H after application.

Evaluation criteria: Trans Epidermal Water Loss (Tewameter®) value &Cutaneous Hydration (Corneometer®) arbitrary unit

Results: Statistically significant effect on the moisturization degree of the upper layers of the epidermis 1 h (+39%-+65%), 4 h (+19%-+41%), 8 h (+16%-+36%) and 24 h (+6%-+17%) after the single standardized application was observed as compared to Baseline and to a non-treated area. The results demonstrated that the instant personal care compositions continue to hydrate the skin up to 24 hours. Statistically significant effect on the TEWL was (−14%), 1 h after the application was observed as compared to Baseline and to a non-treated area. The results demonstrated that the instant personal care compositions protect/respect/maintain the skin barrier.

### In Vivo Anti-Oxidant Study

Number of subjects: 10 females with phototype II to IV skins. Average age: 40 y.o. [24-54].

Method: Evaluation of an antioxidant efficacy assessed by comparing the difference on squalene content between non-treated area and treated area after oxidative stress (modelized by 15 min exposure to cigarettes smoke). Squalene content should be higher on treated area if the composition shows antioxidant effect.

Evaluation criteria: Squalene concentration (μg/mg)

Results: Statistically significant higher quantity of squalene concentration was observed in the Treated/Exposed group compared to the Non-Treated/Exposed group. These results show the antioxidant effect of the tested product. The Protection Index was calculated to be between 32.6% and 36.1%.

### Deep Cleansing/Anti-Pollution Study

Number of subjects: 22 females with phototype I to IV skin. Average age: 34 y.o. [18-60].

Method: Evaluation of the cleansing capacity of the instant personal care composition against particles used as a model of pollution, versus a control area cleansed with water only.

Evaluation criteria: Quantity of particles removed after cleansing and calculation of a cleansing capacity.

Results: Statistically significant higher quantity of micro-particles were removed on the zone cleansed with the instant personal care composition than on the zone cleansed with water shows the instant composition's capacity against microparticles used as a model of pollution.

Statistically significant cleansing capacity was observed against microparticles used as a model of pollution. Given the cleansing capacity (C %=+44%) of the instant personal care composition, it can be considered to have a deep cleansing capacity.

### Make Up Remover Effect (for Cleansing Water)

Number of subjects: 21 females with phototype: I to IV skin. Average age: 46 y.o. [20-60].

Method: Evaluation of the make-up removal efficacy of the cleansing water.

Evaluation criteria: Grey level measured via image analysis, using an image processing software (e.g., Photoshop).

Results: After foundation deposit (t1), the data analysis showed a statistically significant decrease of “grey level” in comparison with nude skin (t0) (p<0.0001). After cleansing with the instant personal care composition (t2), the data analysis showed a statistically significant increase of “grey level” in comparison with after foundation deposit (t1) (p=<0.0001).

The product induced 84% of clean skin level after a foundation standardized application.

### Barrier Recovery Study (Repairing Effect)

Number of subjects: 25 female healthy. Average age: 47.7 y.o. [18-60] with phototypes and skin origin as follows: I: 5 (20.00%) II: 10 (40.00%) III: 9 (36.00%) IV: 1 (4.00%) V: 0 (0.00%). Forearm skin types: D0: Body (forearm) 2 mg/cm² on 40 cm² by trained technician D1 to D14: at home application—once daily, subject to apply thin layer of product to designated site.

Method: on site on DO then once a day, by the subject herself, under normal conditions of use, on the dedicated area located to one forearm. Two areas were designated, one on each forearm: one control, one treated. Alteration of the skin barrier function with tape strippings on D0T0, until reaching a TEWL measurement greater than 20 g/m²/h.

Measurement of the TransEpidermal Water Loss (T.E.W.L.) was done on D0T0 (before stripping) (baseline), D0T0′ (immediately after strippings), D3, D7 and D14.

Evaluation criteria: Trans Epidermal Water Loss was measured by Evaporimeter®. There was no statistically significant difference in TEWL value between the untreated and treated site at baseline (pre-tape stripping). There was no statistically significant difference in TEWL value between the untreated and treated site at baseline (post-tape stripping).

Evaluating treated and untreated sites at all post-baseline time points, there was a statistically significant decrease (improvement) in TEWL value, indicating improvement in skin barrier repair, for both the treated and untreated sites when compared to baseline post-tape stripping measurements.

Comparing the treated vs. untreated site, treatment differences, based on the change from immediate post-tape stripping, the treated site demonstrated greater improvement in skin barrier function repair (TEWL) values at the Immediate (15 minute) time point.

Comparing the treated vs. untreated site, the treated site demonstrated a significantly better recovery effect at the immediate (15 minute) time point. By Day 3, there was no significant difference between the treated site and the pre-stripped baseline value, compared to the untreated site.

Under the conditions of the study, a single treatment with the test product Composition 5 repaired skin barrier function at immediate (15 minute) time point evaluated. Under the conditions of the study, a once a day treatment with the test products Composition 5 or Composition 3 demonstrated a skin barrier recovery effect by Day 3.

### Example 4: Detailed Results of the Clinical Tests

### Composition 1

Composition 1 clinical results are summarized in Table 2.

TABLE 2
Composition 1 Clinical Results

| SCIENTIFIC CLAIM | SUBSTANTIATION | RESULTS & COMMENTS |
|---|---|---|
| Efficacy of cleansing: | Deep cleansing measurement + | Improved cleansing capacity |
| completely removes make-up, | Make up remover test + | (C % = +44%) versus water |
| deeply cleansed and hydrated, | subjective questionnaire | Induced 84% of clean skin level |
| gently cleanses, skin cleaned | | after a foundation standardized |
| of make-up, oil and dirt, keep | | application |
| skin hydrated while cleansing, | | After 4 weeks >93% |
| skin feels purified & detoxified | | |
| Hydration: immediate/instant | Corneometry measurement + | +39.20% at 1 h (and 6.59% at |
| effect | subjective questionnaire | 24 h) |
| Skin feels deeply cleansed and | | All items >95% |
| hydrated, feels boosted in | | |
| hydration, keeps the skin | | |
| hydrated while cleansing, | | |
| product does not dry out my | | |
| skin | | |
| Sensitive skin: helps to | Subjective skin sensitivity | After 4 weeks >95% |
| decrease skin sensitivity, skin | questionnaire + | Soothing effect −94% |
| is more comfortable, less | Sensicale | |
| reactive, looks healthier | | |
| Skin barrier: | TEWL measurement | −14.07% at 1 h |
| immediate/instant | | |
| strengthening effect | | |
| Healthy & youthful skin: | Clinical grading + | After 4 weeks on Asian panel: |
| skin looks visibly radiant, | Subjective questionnaire | skin luminosity (+17%), |
| more supple, firmer, feeling | | radiance (+8%), texture (+4%), |
| provides a healthy glow, helps | | firmness (+18%), elasticity |
| even and smooth skin's | | (+16%), suppleness (+7%) |
| texture, looks | | After 4 weeks >93% |
| brighten/illuminated, pores | | |
| look unclogged, skin feels like | | |
| depolluted/detoxified | | |

### Composition 2

The cutaneous acceptability of Composition 2 was judged, on the whole, good, after repeated applications under normal conditions of use, once a day, for 4 consecutive weeks, to the whole face, by 39 female adult subjects, from 31 to 55 years old, with a “sensitive” face skin of all types, with a phototype I to IV, and presenting with the predefined inclusion criteria.

In addition, the single controlled application of the investigational product, led to a statistically significant improvement in wrinkles severity (Crow's feet area), skin radiance and in skin luminosity, immediately after application of the investigational product, in comparison with the initial evaluations, on the basis of clinical evaluation performed by a Dermatologist Investigator.

Moreover, the repeated applications of the investigational product performed once a day by the same subjects, during 4 weeks, led to:
a. a statistically significant improvement in all evaluated criteria (except in skin elasticity and in skin suppleness/mobility) after 2 weeks of use, and in all evaluated criteria (except in skin suppleness/mobility) after 4 weeks of use, in comparison with the initial evaluations, on the basis of clinical evaluation performed by a Dermatologist Investigator;
b. a statistically significant and favorable decrease in the global score obtained with the Sensiscale (sensitive scale) questionnaire, after 4 weeks of use, in comparison with the initial evaluations, showing into evidence a decrease of the skin sensitivity, on the basis of self-assessment scales filled in by the subjects; a statistically significant and favorable increase in the global score obtained with the “Healthy aging score” questionnaire, after 4 weeks of use, in comparison with the initial evaluations, on the basis of self-assessment scales filled in by the subjects;
c. a statistically significant decrease in Rt (maximum amplitude), Rm (maximum roughness), Rz (average roughness), Rp (smoothness—depth) and Surface (developed surface percentage) parameters after 2 and 4 weeks of use, with in addition statistically significant decrease in Volume (V/mm²) parameter after 4 weeks of use, in comparison with the initial values, showing into evidence a smoothing effect (effect on fine lines), on the basis of replica analysis (Quantilines™ software).

Besides, the cosmetic qualities and the efficacy of the product were well and quite well appreciated by the subjects as respectively 85% and 67% of them judged its cosmetic acceptability and its efficacy as “good” to “very good.”

Composition 2 clinical results are summarized in Table 3.

TABLE 3
Composition 2 Clinical Results

| SCIENTIFIC CLAIM | SUBSTANTIATION | RESULTS & COMMENTS |
|---|---|---|
| Healthy & youthful skin: | Clinical grading + | After 4 weeks on Asian panel: |
| feels revitalized, visibly | Subjective questionnaire | skin luminosity (+14%), radiance |
| radiant, provides a healthy | | (+27%), evenness (+13%), texture |
| glow, feels healthier, looks | | (+16%), firmness (+28%), |
| younger, less fatigue, | | elasticity (+22%), suppleness |
| improves uneven skin & | | (+25%), wrinkles (−27%), fines |
| dullness, looks | | lines (−43%) |
| brightened/illuminated, fine | | After 4 weeks >93% |
| lines/wrinkles look smoothed, | | |
| firmer, supple, more elastic, | | |
| replenished/plumped | | |
| Hydration: | Corneometry measurement + | +52% at 1 h and +9% at 24 h |
| immediate/instant effect and | Subjective questionnaire | +7% after 4 weeks |
| long term moisturizing effect | | After 4 weeks = 95% |
| The skin feels boosted in | | |
| hydration | | |
| Skin barrier: long term | TEWL measurement + | After 4 weeks −11% TEWL |
| effect only | Subjective questionnaire | |
| (Kinetic 24 h → no change/ | | |
| respect/maintain skin | | |
| barrier): Skin looks repaired | | |
| & protected (100%) | | |
| Sensitive skin: helps to | Subjective skin sensitivity | After 4 weeks >93% |
| decrease skin sensitivity, skin | questionnaire + | Soothing effect −65% |
| is more comfortable, less | Sensicale | |
| reactive, looks healthier | | |

### Composition 3

Composition 3 clinical results are summarized in Table 4.

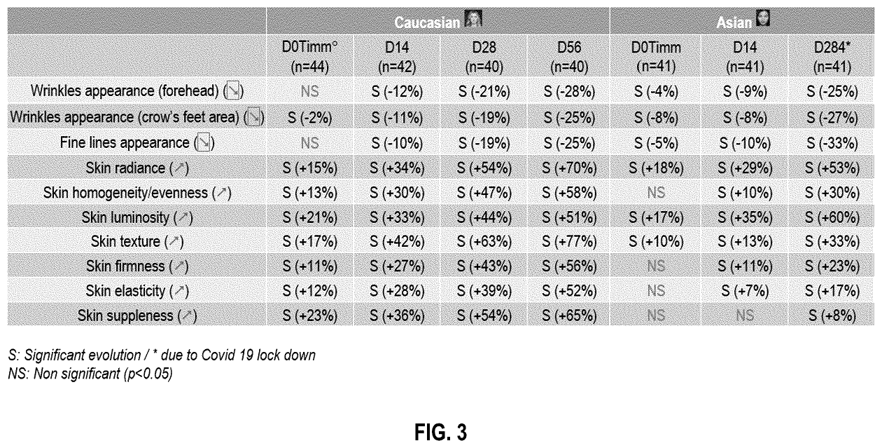

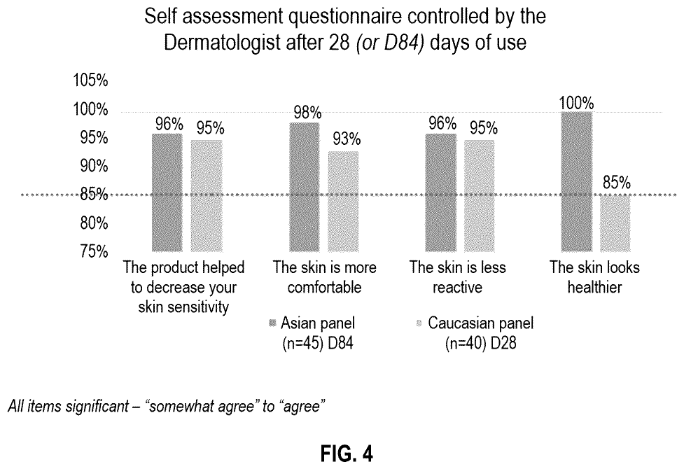

TABLE 4
Composition 3 clinical results.

| a | SUBSTANTIATION | RESULTS & COMMENTS |
|---|---|---|
| Healthy & youthful skin: | Clinical grading + | After 4 weeks on Asian panel (see |
| feels revitalized, visibly | Subjective questionnaire | FIG. 3): skin luminosity (+60%), |
| radiant, provides a healthy | | radiance (+53%), homogeneity |
| glow, looks younger, | | (+30%), texture (+33%), firmness |
| improves uneven skin & | | (+23%), elasticity (+17%), |
| dullness, looks | | suppleness (+8%), wrinkles (−27%), |
| brightened/illuminated, fine | | fines lines (−33%) |
| lines, smoothed, texture | | After 4 weeks >91% |
| appears smoother, firmer, | | |
| more supple, more elastic, | | |
| look rejuvenated, feels | | |
| replenished/plumped | | |
| Hydration: | Corneometry measurement + | +52.76% at 1 h and +10.4% at 24 h |
| immediate/instant effect | Subjective questionnaire | After 4 weeks >93% |
| Skin feels intensely and | | |
| optimally hydrated or | | |
| rehydrated, feels boosted in | | |
| hydration | | |
| Skin barrier: immediate | TEWL measurement + | −12.65% at 1 h |
| strenghtening effect. | Subjective questionnaire + | After 4 weeks >93% |
| Skin looks repaired, feels | Barrier recovery test | −8% TEWL after 10 days of use |
| protected. | | (protective effect of the product, |
| | | improvement in the skin barrier on |
| | | stripped skin) |
| Sensitive skin: helps to | Subjective skin sensitivity | After 4 weeks >96% |
| decrease skin sensitivity, | questionnaire (see FIG. 4) + | Soothing effect −83% |
| skin is more comfortable, | Sensicale | |
| less reactive, looks healthier | | |

### Composition 4

Composition 4 clinical results are summarized in Table 5.

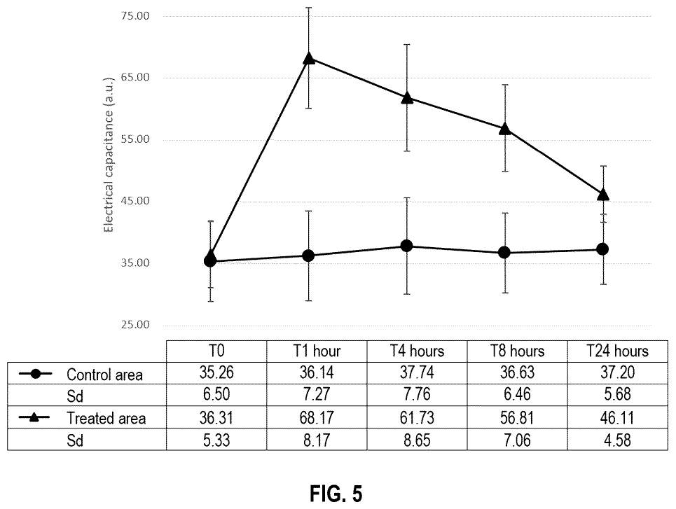

TABLE 5
Composition 4 clinical results.

| SCIENTIFIC CLAIM | SUBSTANTIATION | RESULTS & COMMENTS |
|---|---|---|
| Healthy & youthful skin: | Clinical grading + | On Caucasian panel after 4 weeks: |
| Caucasian & Asian panel: | Subjective questionnaire | skin luminosity (+16%), |
| >75% skin looks more even, | | radiance (+16%), homogeneity |
| looks & feels healthier, appears | | (+10%), texture (+10%), firmness |
| firmer, more supple, feels | | (+5%), dark circles (−9%), |
| strengthened/more resilient, | | wrinkles (−5%), fines lines (−6%) |
| feels nourished, product | | On Asian panel after 4 weeks: |
| improves the skin quality | | skin luminosity (+7%), radiance |
| appearance overtime, soothes | | (+6%), homogeneity (+5%), |
| the skin around the eyes, | | texture (+10%), dark circles (−9%), |
| suitable for sensitive skin | | wrinkles (−12%), fines lines |
| around the eyes | | (−16%) |
| Only on Asian panel: provides | | After 4 weeks >75% Caucasian |
| a “healthy glow”, looks | | & Asian panels |
| brighter/illuminated, looks | | |
| visibly radiant, more youthful, | Instrumental crow's feet | On Caucasian panel after 4 |
| fines lines & wrinkles look | wrinkles measurement - | weeks |
| reduced/smoothed, looks | Relief analysis | wrinkles number (−13.1%), total |
| plumped, restores skin | | wrinkle area (−12.8%), total |
| elasticity, reduces dark circles | | length (−18.6%) |
| & signs of fatigue | | |
| Sensitive skin: helps to | Subjective skin sensitivity | After 4 weeks >72% on |
| decrease skin sensitivity, skin | questionnaire + | Caucasian panel |
| is more comfortable, less | Sensicale | Soothing effect −62% on |
| reactive (60% NS), looks | | Caucasian panel |
| healthier | | Soothing effect −68% on Asian |
| | | panel |
| Hydration: immediate/instant | Corneometry measurement + | +85% at 1 h and +21% at 24 h (see |
| effect | Subjective questionnaire | FIG. 5) |
| skin feels hydrated, stays | | After 4 weeks >93% Asian and |
| hydrated | | caucasian panel |
| Skin barrier: | TEWL measurement + | −20% at 1 h |
| immediate/instant | Barrier recovery test/ | Didn't lead to any significant |
| strenghtening effect | repairing effect | difference |

### Composition 5

Composition 5 clinical results are summarized in Table 6.

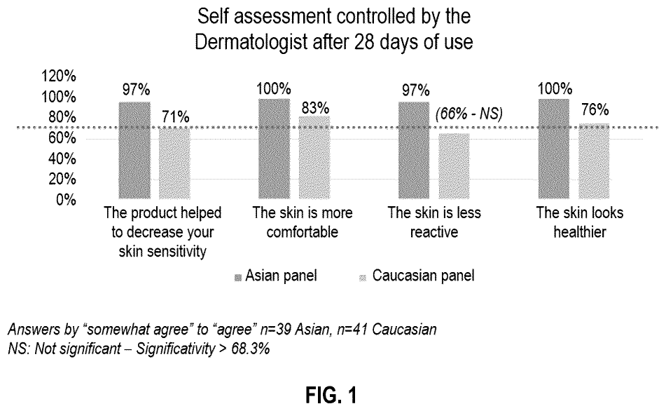

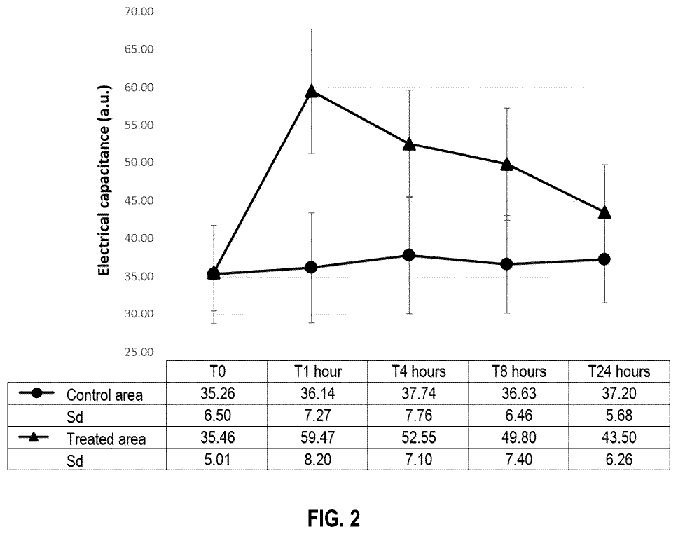

TABLE 6
Composition 5 clinical results.

| SCIENTIFIC CLAIM | SUBSTANTIATION | RESULTS & COMMENTS |
|---|---|---|
| Healthy & youthful skin: | Clinical grading + | After 4 weeks on Asian panel: skin |
| leaves the skin feeling firm | Subjective questionnaire | luminosity (+22%), radiance |
| and smooth, feels nourished/ | | (+24%), texture (+14%), firmness |
| nurtured, revitalized, fresh | | (+16%), elasticity (+15%), |
| healthy-glow, restores | | suppleness (+16%), wrinkles |
| healthy-looking skin, looks | | (−13%), fines lines (−15%) |
| and feels healthier, leaves | | After 4 weeks >93% |
| the skin looking younger, | | |
| improves uneven skin & | | |
| dullness, looks | | |
| brightened/illuminated, | | |
| softer, firmer, more supple, | | |
| more elastic, look | | |
| rejuvenated, feels | | |
| replenished/plumped, | | |
| diminishes the visible signs | | |
| of fatigue, feels the skin is | | |
| strenghtened/more resilient | | |
| to fight against premature | | |
| aging | | |
| Hydration: | Corneometry measurement + | +65.21% at 1 h and 17.17% at 24 h |
| immediate/instant effect | Subjective questionnaire | (see FIG. 2) |
| Skin feels intensely and | | After 4 weeks on Asian panel >98% |
| optimally hydrated or | | |
| rehydrated (100%), after | | |
| wake-up, skin feels | | |
| completely nourished, | | |
| hydrated and plumped (98%) | | |
| Skin barrier: (Kinetic 24 h → | TEWL measurement + | No change in TEWL (kinetic 24 h) |
| no change/respect/ | Subjective questionnaire + | After 4 weeks >100% on Asian |
| maintain skin barrier) | Barrier recovery test/ | panel |
| Skin looks repaired, feels | repairing effect | −7% TEWL after 10 days of use |
| protected. | | (protective effect of the product, |
| | | improvement in the skin barrier on |
| | | stripped skin) |
| Sensitive skin: helps to | Subjective skin sensitivity | After 4 weeks >97% |
| decrease skin sensitivity, | questionnaire (see FIG. 1) + | Soothing effect −98% |
| skin is more comfortable, | Sensicale | |
| less reactive, looks healthier | | |

The clinical trials showed that, upon using the personal care compositions described herein, the following benefits were commonly observed:

### Healthy & Youthful Skin

Skin feels revitalized, nourished/nurtured, skin looks healthier (↑ radiance, texture, firmness, elasticity, suppleness, wrinkles, fines lines)
Skin looks visibly radiant and luminous, brightened/illuminated.
Provides a fresh “healthy glow”: Restores healthy-looking skin.
Leaves the skin looking younger (improvement of texture, firmness, elasticity, suppleness, wrinkles, fines lines)
Improves uneven skin and dullness. Skin texture appears smoother.
Skin looks firmer, more supple, feels more elastic, replenished/plumped. Fines lines look reduced/smoothed
Improves skin quality appearance overtime
Helps to diminish the visible signs of fatigue (improvement of texture, firmness, elasticity, suppleness, wrinkles, fines lines)
Skin is strengthened/more resilient to fight against premature aging.

### Skin Barrier:

Skin looks repaired and protected. More resilient to fight against premature aging (100% from questionnaire).
Protective effect/improvement in the skin barrier after 10 days of product use on compromised skin.

### Hydration

Instantly hydrates the skin and keeps it hydrated during 24 h (see FIG. 2).
Skin feels completely nourished, hydrated and plumped.

### Sensitive Skin

Suitable for sensitive skin. Has soothing effect. Soothes the skin.
Helps to decrease skin sensitivity, skin is more comfortable, less reactive (see FIG. 1).

### Example 5: Mitochondrial Studies

Environmental assaults such as UV light and pollution facilitate skin aging. Phenotypic manifestation of skin aging includes wrinkle formation, dryness, loss of elasticity, suppleness, firmness, luminosity, hyperpigmentation and increase in sensitivity of the skin. At the cellular level, it is believed that the mitochondria, the cellular bioenergetic factory, is a key player in aging. Biochemically, it has been observed that the mitochondria of aged skin exhibit an increase in NADPH-oxidase, decrease in mitochondrial membrane potential (MMP), an unbalanced fusion-fission of fragmented mitochondrial and an increase in mitochondrial associated endoplasmic membranes (MAMs). The effect of the disclosed personal care compositions on mitochondrial function and protection was tested as described below.

TABLE 7
Below is an exemplary personal care composition (Composition
A) that is used in the mitochondrial assays.

| Ingredient | Amount |
|---|---|
| Edelweiss extract | 1.50% |
| ALPAFLOR ® NECTAPURE PF | 0.01% |
| Buddleja davidii extract + Thymus | |
| vulgaris extract (manuf. solution) | |
| Niacinamide | 1.50% |
| Adenosine | 0.04% |
| Pro-vitamin B5 (panthenol) | 2.50% |
| Rice ferment (from Koji) extracts (rice | 1.00% |
| (Oryza sativa) lees concentrate) | |

Composition A was tested at 1/200 (0.5%), 1/2,000 (0.05%) and 1/20,000 (0.005%) dilutions (compared to the concentrations listed in Table 7 above) in DMSO. Finally, 0.005% dilution was selected for all the mitochondrial experiments because this dilution had the least effect on cell viability.

Cells used in the study: a fibroblast culture from a mature donor (54 years old) validated for pollution susceptibility and mitochondrial poisoning.

Pollutant: urban standardized particles composed with metals, polyaromatic hydrocarbons (PAHs), nitro-PAHs, PCBs, chlorinated pesticide, etc. of micrometric diameter.

NADPH-oxidase activity, Mitochondrial membrane potential (MMP), Fusion-fission balance, Mitochondrial associated Membranes (MAMs), and cell viability were measured. The results of these assays were as follows.

### NADPH-Oxidase Activity

NADPH oxidase is an enzyme catalyzing the production of superoxide anion. Dormant in resting cells and activated upon stress. NADPH oxidase activity generates reactive oxygen species (ROS) involved in different physiological mechanisms. Upon pollution, NADPH oxidase is highly stimulated and produces a large quantity of ROS. It leads to the generation of the skin inflammatory, notably the release of the sensitive skin mediator PGE-2. Deactivation of the pollutant and/or regulation of the NADPH oxidase preserves the cells from oxidative damages.

Method: Cells are incubated for 30 minutes with the test elements (either pollutant or Composition A). NADPH oxidase activity is determined immediately using chemioluminescence. Conditions: Untreated and unexposed to pollution, Untreated and exposed to pollution and Treated with personal care composition and exposed to pollution.

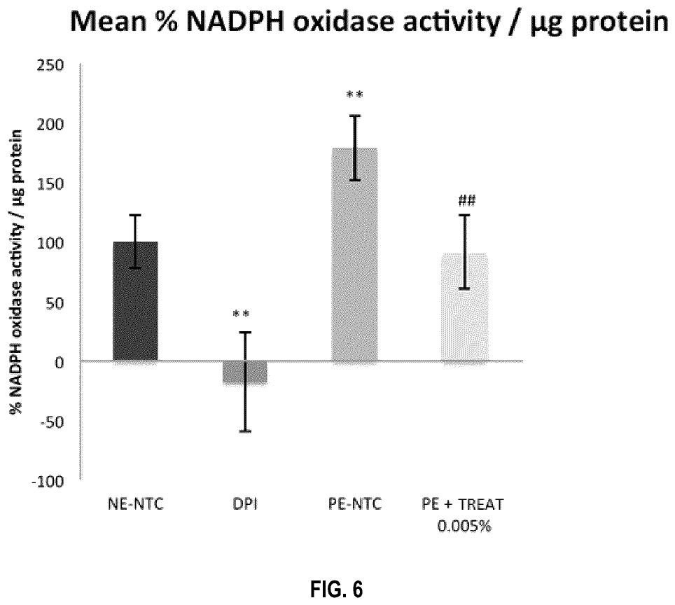

Results: Pollution induced NADPH oxidase activity (p=0.0041) (FIG. 6, PE-NTC), whereas Composition A significantly (p=0.0053) inhibited the pollution-induced NADPH oxidase activity (FIG. 6, PE+TREAT). These results showed the anti-oxidant and pollution protective effects of Composition A.

### Mitochondrial Membrane Potential

Mitochondrial membrane potential (MMP) is the main driving force for ATP synthesis (the cellular fuel). Drops of the MMP (depolarization) can be a sign of damaged mitochondria by oxidative stress and is observed during aging. Pollution-induced depolarization leads to a reduced production of ATP (cellular fuel) and, ultimately, to premature aging.

Method: Cells are preincubated with a probe. Then, cells are incubated for 4 h with the test elements (either pollutant or actives). MMP is recorded immediately using chemioluminescence. Conditions: Untreated and unexposed to pollution, Untreated and exposed to pollution and Treated with Composition A and exposed to pollution.

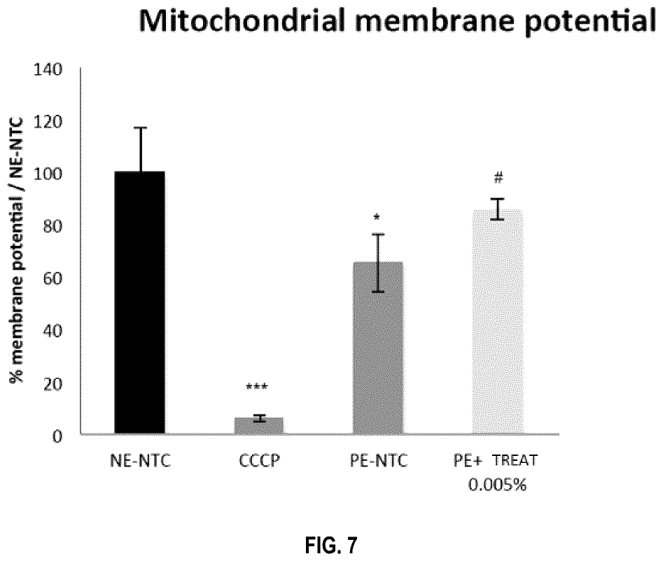

Results: Pollution induced mitochondrial depolarization (−34%, p=0.0151), and the personal care product significantly alleviated the pollution's effects (+20%, p=0.0145) (FIG. 7). Upon treatment with the personal care product, the mitochondrial membrane potential comes back to a level which is not significantly different from the basal condition. Composition A sustains the cell energy factory upon pollution to support the normal function of the mitochondrion.

### Fission-Fusion Balance

The mitochondrial network is a balance between elongated and fractioned forms of mitochondria. This balance is dynamic to insure a healthy mitochondrial activity. Stress, like pollution, can damage some mitochondria. It is crucial for the cells to remove the damaged ones and create new ones in a balanced way. Unbalance of the mitochondrial network appends in different conditions and disease. A hallmark of aging is a shift toward fragmented mitochondria.

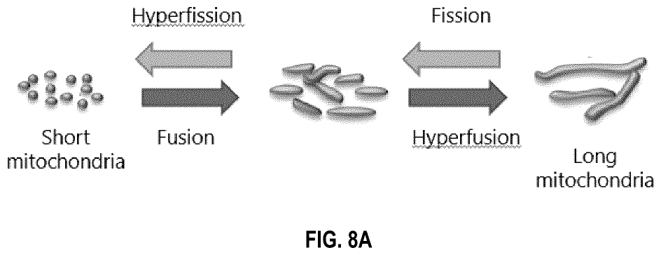

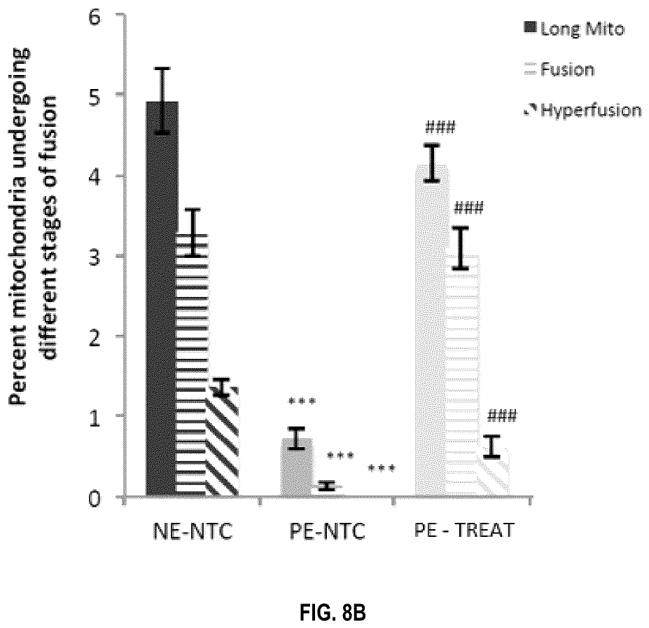

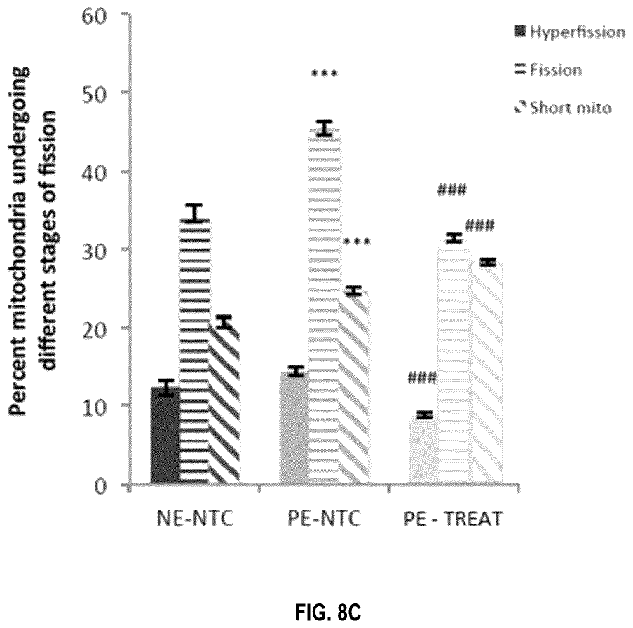

Methods: Cells are preincubated with a probe. Then, cells are incubated overnight with the test elements (either pollutant or actives). Mitochondrial fusion parameters (FIG. 8A) are measured immediately using Mitosteam Technology. Conditions: Untreated and unexposed to pollution, Untreated and exposed to pollution and Treated with Composition A and exposed to pollution.

Results: Pollution blocks mitochondrial fusion. Hyperfusion disappeared upon pollution exposure (p<0.0001) (FIG. 8B). There was a 96% decrease in fusion (p<0.0001), and a 86% decrease in long mitochondria (p<0.0001) (FIG. 8B). Upon treatment with Composition A, the pollution-induced decrease in mitochondrial length and complexity were rescued: Hyperfusion was restored back to normal levels. There was a 2156% increase in fusion (p<0.0001), and a 475% increase in long mitochondria (p<0.0001) (FIG. 8B).

Pollution induces a shift toward fission of the mitochondrial network: +17% increase in hyperfission (p=0.06), 31% increase in fission (p<0.0001), and a 20% increase in short mitochondria (p<0.0001) was observed (FIG. 8C). Upon treatment with Composition A, the pollution-induced increase in mitochondrial fission was reversed back to normal. There was a 22% decrease in hyperfission (p<0.0001), a 21% decrease fission (p<0.0001) and a 15% increase in short mitochondria (p<0.0001) (FIG. 8C).

### Mitochondrial Network

Next, Composition A was tested on a mitochondrial network of cells. Pollution induced a collapsed mitochondrial network, whereas treatment with Composition A preserved the complexity and branching of the mitochondrial network upon pollution challenge.

Composition A preserves the complexity and branching of mitochondrial population and prevents pollution-induced damages to the mitochondrial network and protects skin from premature aging.

### Mitochondria-Associated Endoplasmic Reticulum Membranes (MAMs)

Organelles are in close vicinity in the cell. MAMs are highly specialized domains between endoplasmic reticulum (ER) and mitochondria. Urban pollution leads to an increase of MAMs through a stress to the endoplasmic reticulum. MAMs are the sites of formation of the inflammasome. Inflammasome formation is increased in presence of damaged mitochondria and contribute to aging through the process of “inflammaging.”

Methods: Cells are preincubated with a probe. Then, cells are incubated overnight with the test elements (either pollutant or Composition A). Ligation assay measured the distance between GRP75 (mitochondrial membrane) and SERCA2 (endoplasmic reticulum)—distance<40 nm was considered a positive result. Conditions: Untreated and unexposed to pollution, Untreated and exposed to pollution and Treated with Composition A and exposed to pollution.

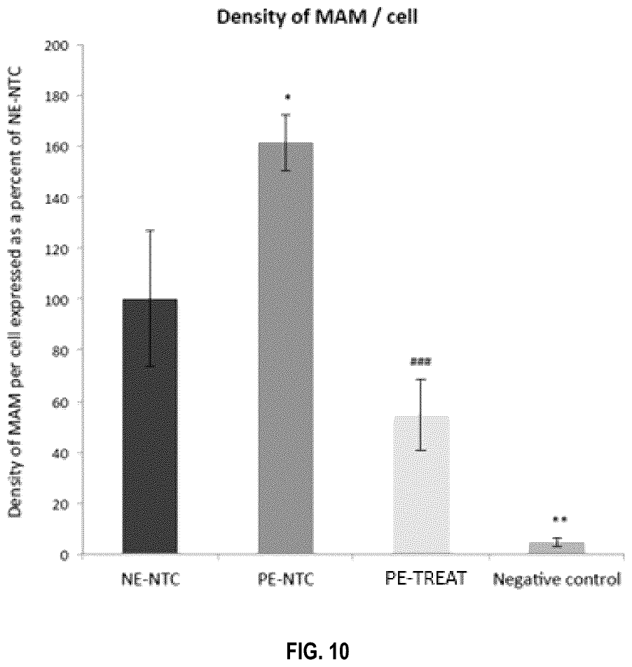

Results: The personal care composition significantly (p<0.0001) decreased the number of MAMs in cell injured by pollution by 66% (FIG. 10).

### Cell Viability

The effect of Composition A was tested to assess cell viability.

Methods: Cells were incubated overnight with the test elements (either pollutant or actives). Cell number was determined per microscopic fields. Conditions: Untreated and unexposed to pollution, Untreated and exposed to pollution and Treated with Composition A and exposed to pollution.

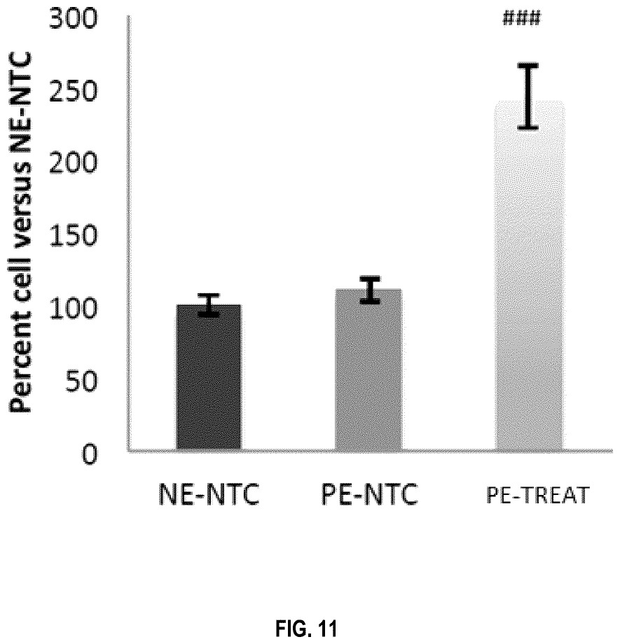

Results: Pollution did not affect the number of cells (p=0.30). Despite pollution, Composition A boosted cell number by 118% (p<0.0001) (FIG. 11).

In conclusion, Composition A counteracts multiple signs of pollution-induced mitochondrial defects, which are also observed during aging.

### Example 6: Mitochondrial Studies on Personal Care Composition Comprising Soy-Derived Glycopeptides

Biochemically, it has been observed that the mitochondria of aged skin exhibit an increase in NADPH-oxidase enzyme activity, decrease in mitochondrial membrane potential (MMP), an unbalanced fusion-fission of fragmented mitochondria, and an increase in mitochondrial associated endoplasmic membranes (MAMs). The effect of the disclosed personal care compositions (see Table 8) on mitochondrial function and protection was tested as described below.

TABLE 8
Exemplary personal care composition (Composition
B) for mitochondrial assays.

| Ingredient | Amount |
|---|---|
| Edelweiss extract (manuf. solution) | 3.0% |
| Hydrolyzed soy flour concentrate (in | 2.0% |
| water, caprylyl glycol, and 1,2- | |
| hexanediol) (manuf. solution) | |
| Niacinamide | 1.50% |
| Pro-vitamin B5 (panthenol) | 2.50% |
| Rice ferment (from Koji) extracts (rice | 1.00% |
| (Oryza sativa) lees concentrate) | |
| (manuf. solution) | |

As a comparison to the personal care composition, niacinamide was tested at low concentration (“niacinamide (low)”) and high concentration (“niacinamide (high)”) in DMSO. (The high niacinamide concentration was 6.67 times the low niacinamide concentration.) Composition B was diluted until its niacinamide concentration was the same as the niacinamide (low) concentration. The cells used in the study were human primary fibroblasts originating from a healthy 55-year-old female donor. As in Example 5, the pollutant was urban standardized particles composed with metals, polyaromatic hydrocarbons (PAHs), nitro-PAHs, PCBs, chlorinated pesticide, etc., of micrometric diameter.

NADPH-oxidase activity and mitochondrial associated membranes (MAMs) were experimentally determined and quantified in the same manner as in Example 5. The results of these assays were as follows.

### NADPH-Oxidase Activity

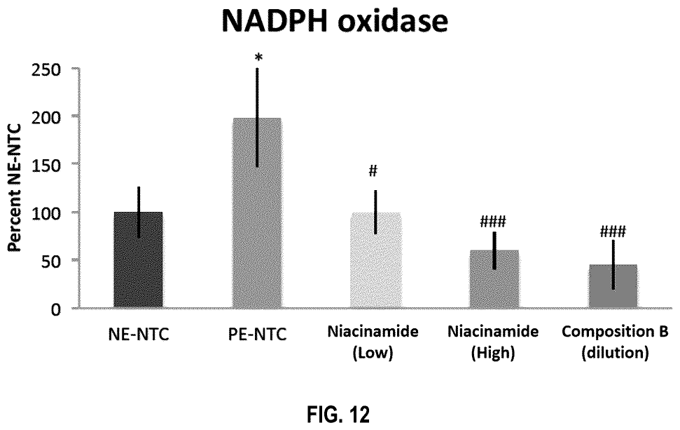

Results: The data presented in FIG. 12 shows a clear antioxidant effect for niacinamide (low), niacinamide (high), and Composition B (diluted), with niacinamide (low) restoring NADPH activity to a level similar to that observed in the NE-NTC. Both niacinamide (high) and Composition B (diluted) reduce NADPH oxidase activity to a level below that observed in NE-NTC.

### Mitochondria-Associated Endoplasmic Reticulum Membranes (MAMs)

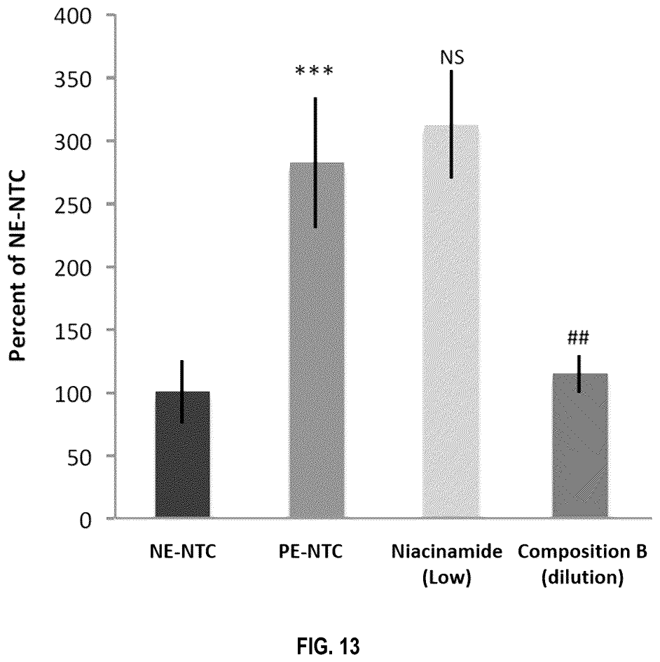

Results: As shown in FIG. 13, pollution increased the MAM density in exposed cells, consistent with increased endoplasmic reticulum stress and increased storage of intracellular calcium in the mitochondria. This effect was reduced to a normal level with Composition B (diluted), on par with the level observed in NE-NTC. The personal care composition (Composition B) significantly (p<0.0001) decreased the number of MAMs in cells injured by pollution by approximately 60%.

In conclusion, Composition B efficiently reduces NADPH-oxidase activity and the occurrence of MAMs, which are responsible for increase in mitochondrial calcium influx. Thus, Composition B counteracts multiple signs of pollution-induced mitochondrial defects, which are also observed during aging.

What is claimed is:

1. A personal care composition comprising, by weight of the total composition:
0.1%-10% niacinamide;
0.0001%-10% Edelweiss (Leontopodium alpinum) extract;
0.001%-10% rice (Oryza sativa) lees concentrate; and
0.1%-10% panthenol.

2. The personal care composition of claim 1, further comprising 0.001%-5% soy-derived glycopeptides.

3. The personal care composition of claim 1, wherein the personal care composition comprises, by weight of the total composition:
0.1%-4% niacinamide;
0.001%-4% Edelweiss (Leontopodium alpinum) extract;
0.001%-2% rice (Oryza sativa) lees concentrate;
0.1%-3% panthenol; and
0.002%-4% soy-derived glycopeptides.

4. The personal care composition of claim 1, wherein the personal care composition does not comprise any retinoids or retinoid derivatives.

5. The personal care composition of claim 1, further comprising at least one of the following: water, adenosine, a moisturizing agent, a light protective filter, an antioxidant, a preservative, a gelling agent, a solvent, a pH balancing agent, and a surfactant.

6. The personal care composition of claim 5, wherein the moisturizing agent comprises at least one of Buddleja davidii extract, Thymus vulgaris extract, caprylyl glycol, glycerin, 1,2-hexanediol, and butylene glycol.

7. The personal care composition of claim 1, further comprising, by weight of the total composition:
0.01%-2% adenosine;
0.0001%-3% Buddleja davidii extract; and
0.0001%-3% Thymus vulgaris extract.

8. The personal care composition of claim 5, wherein the light protective filter comprises at least one of zinc oxide, titanium dioxide, and iron oxide.

9. The personal care composition of claim 1, wherein the personal care composition is a cream, a lotion, a gel, a mask, a serum, or a spray.

10. A method for treating sensitive skin in a subject, the method comprising:
administering the subject the personal care composition according to claim 1.

11. The method of claim 10, wherein the sensitive skin exhibits at least one unpleasant sensation before the treatment, the at least one unpleasant sensation selected from the group consisting of: tingling, heat, burning, itching, tightness and erythema.

12. The method of claim 10, wherein the administration of the personal care composition results in a reduction in wrinkles or a delay in the onset of wrinkles.

13. The method of claim 10, wherein the personal care composition does not comprise any retinoids or retinoid derivatives.

14. The method of claim 10, wherein the personal care composition comprises, by weight of the total composition:
0.1%-4% niacinamide;
0.001%-4% Edelweiss (Leontopodium alpinum);
0.001%-2% rice (Oryza sativa) lees concentrate;
0.1%-3% panthenol; and
0.002%-4% soy-derived glycopeptides.

15. The method of claim 10, wherein the personal care composition comprises at least one of the following: water, adenosine, a moisturizing agent, a light protective filter, an antioxidant, a preservative, a gelling agent, a solvent, a pH balancing agent, and a surfactant.

16. The method of claim 15, wherein the moisturizing agent comprises at least one of Buddleja davidii extract, Thymus vulgaris extract, caprylyl glycol, glycerin, 1,2-hexanediol, and butylene glycol.

17. The method of claim 15, wherein the light protective filter comprises at least one of zinc oxide, titanium dioxide, and iron oxide.

18. The method of claim 10, wherein the personal care composition is administered topically.

19. The method of claim 10, wherein the subject has been exposed to pollution.

20. A method for reducing the onset of wrinkles in a subject with sensitive skin, reducing pollution-induced NADPH oxidase in mitochondria in human skin, reducing pollution-induced fragmentation of mitochondria in human skin, reducing pollution-induced production of MAMs in mitochondria of human skin, alleviating pollution induced mitochondrial membrane depolarization, inducing pro-collagen and elastin protein expression in skin, or reducing IL-8 and PGE-2 expression in skin, the method comprising:
administering to the subject the personal care composition according to claim 1.
